(19) United States
(12) Patent Application Publication
(10) Pub. No.: US 2023/0311097 A1
(43) Pub. Date: Oct. 5, 2023

(54) CATALYSTS AND METHODS OF MAKING AND USE THEREOF

(71) Applicant: Ohio State Innovation Foundation, Columbus, OH (US)

(72) Inventors: Joshua Goldberger, Columbus, OH (US); Kelsey Hodge, Columbus, OH (US)

(21) Appl. No.: 18/022,804

(22) PCT Filed: Jun. 25, 2021
(86) PCT No.: PCT/US21/39060

§ 371 Date: Feb. 23, 2023

Related U.S. Application Data
(60) Provisional application No. 63/070,595, filed on Aug. 26, 2020.

(51) Int. Cl. B01J 23/08; B01J 23/14; B01J 35/10; B01J 35/00; B01J 35/02; C07C 5/09

(52) U.S. Cl. CPC B01J 23/08; B01J 23/14; B01J 35/1019; B01J 35/1014; B01J 35/1009; B01J 35/0006; B01J 35/0013; B01J 35/023; C07C 5/09; C07C 2523/08; C07C 2523/14

(57) ABSTRACT

Disclosed herein are catalysts and methods of making and use thereof, wherein the catalysts comprises a layered inter-metallic compound.

### CROSS-REFERENCE TO RELATED APPLICATIONS

This application claims the benefit of priority to U.S. Provisional Application No. 63/070,595 filed Aug. 26, 2020, which is hereby incorporated herein by reference in its entirety.

### STATEMENT OF GOVERNMENT SUPPORT

This invention was made with government support under Grant No. 1420451 awarded by the National Science Foundation. The government has certain rights in the invention.

### BACKGROUND

It is estimated that between 10-20% of all industrial catalytic processes performed today are hydrogenations in some form. These are typically performed heterogeneously using solid-state compounds that contain Pd, Pt, or other rare and expensive precious metals. The semihydrogenation of C≡C triple bonds into C═C double bonds and full hydrogenation into C—C single bonds is an industrially significant heterogeneously catalyzed reaction, yet it is performed almost exclusively using palladium catalyst. The heterogeneous catalysis of petroleum feedstocks into functionally useful derivatives using inexpensive and earth abundant elements remains an important goal. The compositions, methods, and systems discussed herein addresses these and other needs.

### SUMMARY

In accordance with the purposes of the disclosed compositions, methods, and systems as embodied and broadly described herein, the disclosed subject matter relates to catalysts and methods of making and use thereof.

Additional advantages of the disclosed compositions, systems, and methods will be set forth in part in the description which follows, and in part will be obvious from the description. The advantages of the disclosed compositions, systems, and methods will be realized and attained by means of the elements and combinations particularly pointed out in the appended claims. It is to be understood that both the foregoing general description and the following detailed description are exemplary and explanatory only and are not restrictive of the disclosed systems and methods, as claimed.

The details of one or more embodiments of the invention are set forth in the accompanying drawings and the description below. Other features, objects, and advantages of the invention will be apparent from the description and drawings, and from the claims.

### BRIEF DESCRIPTION OF THE FIGURES

The accompanying figures, which are incorporated in and constitute a part of this specification, illustrate several aspects of the disclosure, and together with the description, serve to explain the principles of the disclosure.

FIG. 1. Schematic diagram showing conversion of BaGa₂ to BaGa₂H₂. Blue spheres correspond to Ga, Red spheres correspond to Ba, Green spheres correspond to H.

FIG. 2. X-ray diffraction of BaGa₂H₂ sample (red) and BaGa₂ (blue).

FIG. 3. Rietveld analysis confirmed of BaGa₂.

FIG. 4. Rietveld analysis confirmed that the majority of the BaGa₂ phase transformed into BaGa.412 upon reacting it for 7 days at 170° C. under 50 bar H₂.

FIG. 5. Scanning electron micrograph image of ground BaGa₂ powder.

FIG. 6. BaGa₂ catalyzed hydrogenation of phenylacetylene (red) to styrene (blue) and ethylbenzene (green).

FIG. 7. Time dependent conversion of phenylacetylene (red) to styrene (blue) and ethylbenzene (green) using 1 bar H₂, 40° C., 2.4 mL DMF solvent, 0.87 mmol phenylacetylene, and 0.043 mmol BaGa₂ catalyst. The solid lines are the fit kinetics traces assuming both semihydrogenation and full hydrogenation reactions are first order with respect to the phenylacetylene, and styrene, respectively.

FIG. 8. Time dependent conversion phenylacetylene (reed) to styrene (blue) and ethylbenzene (green) at 51 bar H₂, 40° C., 2.4 mL DMF solvent, 1.03 mmol phenylacetylene, and 0.060 mmol BaGa₂ catalyst. The solid lines are the fit kinetics traces assuming both semihydrogenation and full hydrogenation reactions are first order with respect to the phenylacetylene, and styrene, respectively.

FIG. 9. Catalyst recycling experiments over four runs. Reaction conditions: 51 bar H₂, 100° C., 20 hr, 0.058 mmol of BaGa₂ catalyst, 1.1 mmol phenylacetylene, 2.4 mL n-butanol solvent.

FIG. 10. XPS spectra of the Ga 2p_{3/2} and 2p_{1/2} peaks of BaGa2 before (red, upper) and after (blue, lower) exposure to air. * corresponds to the non-oxidized BaGa₂.

FIG. 11. A schematic diagram of Pb absorbing H₂.

FIG. 12, Schematic of hydrogenation catalyzed using the Zintl. phase catalysts described herein.

FIG. 13. Examples of binary and ternary layered Zintl phases.

FIG. 14. An example of a hydrogen absorbing Zintl phases is SrAl₂, which becomes SrAl₂H₂ upon absorption of hydrogen.

FIG. 15. An example of a hydrogen absorbing Zintl phase is SrGa₂/BaGa₂.

FIG. 16. BaGa₂ can absorb H₂ to become BaGa₂H₂.

FIG. 17. BaGa₂ was used to catalyze the hydrogenation of phenylacetylene to styrene.

FIG. 18. Photograph of hydrogenation of phenylacetylene to styrene reaction progress using BaGa₂.

FIG. 19. % Conversion of phenylacetylene to styrene with BaGa₂.

FIG. 20. Analysis of BaGa₂ catalyst.

FIG. 21. Analysis of styrene produced from the hydrogenation of phenylacetylene using BaGa₂ as a catalyst, which gives 93% cis selectivity.

FIG. 22. % Conversion of phenylacetylene to styrene with BaGa₂ using DMF vs. n-butanol/NaOH.

FIG. 23. Mol % phenylacetylene, styrene, and ethylbenzene over time using BaGa₂ catalyst.

FIG. 24. Ease of hydrogenation of various functional groups.

FIG. 25. Crystal structures of phases used as catalysts. In all compounds, the main group network forms a layered honeycomb network of atoms, in which the layers are oriented horizontally, and into the page. Ba/Y/La/Eu are depicted as red spheres, Ca is depicted as green spheres, Ga is depicted as blue spheres, In is depicted as purple spheres, Ge is depicted as either black or gray spheres, and Si is depicted as grey spheres. In BalnGe and CaGaSi, the In/Ge and Ga/Si atoms are positioned randomly on the framework.

FIG. 26. Powder X-ray diffraction pattern of BaInGe.

FIG. 27. Powder X-ray diffraction pattern of CaGaSi.

FIG. 28. Powder XRD pattern of o-EuGa₂. The most intense Eu₃Ga₈ impurity reflections are denoted with asterisks.

FIG. 29. XRD pattern LaGa₂. Asterisks indicate La₂O₃ impurity.

FIG. 30. Powder XRD pattern of CaIn₂. Asterisks indicate reflections correspond to an In impurity.

FIG. 31. Powder XRD pattern of YGa₂. Asterisks correspond to trace Y₂O₃ impurity.

FIG. 32. Powder X-ray diffraction pattern of 13T-CaGaGe.

FIG. 33. Powder X-ray diffraction pattern of h-EuGa₂. Asterisks correspond to reflections from Eu₃Ga₈ impurities.

FIG. 34. Powder X-ray diffraction pattern of 4H-CaGaGe.

FIG. 35. Superimposed X-ray diffraction patterns of 4H-CaGaGe (uppermost trace), 13T-CaGaGe (middle trace), and calculated 13T-CaGaGe (lowest trace).

FIG. 36. Rietveld refinement results of the o-EuGa₂ sample containing a 0.082 phase fraction of Eu₃Gas.

FIG. 37. Rietveld refinement results of the h-EuGa₂ containing 0.078 phase fraction of Eu₃Gas.

FIG. 38. Eu₃Gas crystal structure looking down the b-axis.

FIG. 39. Eu₃Gas crystal structure looking down the a-axis.

FIG. 40. LaGa₂ diffraction pattern after 2 h air exposure showing decomposition of the LaGa₂ phase. Triangles represent Ga₂O₃, asterisks represent La₂O₃, and squares represent La₂(OH)₃.

FIG. 41. Superimposed YGa₂ diffraction before (upper trace) and after (lower trace) air exposure.

FIG. 42. X-ray Photoelectron spectra of the Ga 2p_{3/2} peak of 13T-CaGaGe before (top) and after (bottom) air exposure. The fitting of the reduced Ga⁻ at ˜1115.5 eV, and the more oxidized Ga peaks at ˜1117.1-1117.9 and 1119.8-1120.2 eV.

FIG. 43. X-ray Photoelectron spectra of the Ga 2p_{3/2} peak of YGa₂ before top) and after (bottom) air exposure. The fitting of the reduced Ga⁻at ˜1115.5 eV, and the more oxidized Ga peaks at ˜1117.1-1117.9 and 1119.8-1120.2 eV.

FIG. 44. X-ray Photoelectron spectra of the Ga 2p_{3/2} peak of BaGa₂ before (top) and after (bottom) air exposure. The fitting of the reduced Ga⁻ at ˜1115.5 eV, and the more oxidized Ga peaks at ˜1117.1-1117.9 and 1119.8-1120.2 eV.

FIG. 45. Catalyst recyclability study for 13T-CaGaGe compared to BaGa₂ showing greater that 90% conversion of phenylacetylene to styrene and after seven cycles for the same CaGaGe catalyst at 51 bar H₂, n-BuOH. 90° C., 20 h, 8.1 mol % (starting).

FIG. 46. Catalyst air stability study for CaGaGe after 1 day, 6 days and 5 months of air exposure to the CaGaGe powder (51 bar H₂, n-BuOH, 90° C., 20 h, 6.5-8.1 mol %).

FIG. 47. Superimposed xRD patterns of 13T-CaGaGe pre (upper trace) and post (lower trace) catalysis.

FIG. 48. Time dependent conversion of phenylacetylene to styrene and ethylbenzene in NNW at 40° C., 0.91 mmol phenylacetylene, 2.5 mL NMP, 15 mg 13T-CaGaGe. The specific surface activity was calculated from the 5 h time point which includes the induction period, and thus represents a lower bound.

FIG. 49. Time dependent conversion plot of phenylacetylene to styrene and ethyllbenzene (DMF, 40° C., 8.4 mol % 13T-CaGaGe, 0.91 mmol phenylacetylene, 1 bar H₂, 2.5 mL solvent). Ethylbenzene was below the limit of detection (<0.001%).

FIG. 50. Kr adsorption isotherm of 13T-CaGaGe

FIG. 51. Time dependent conversion using Lindlar's catalyst of phenylacetylene to styrene and ethylbenzene in DMF, 40° C., 17 mg catalyst, 0.91 mmol phenylacetylene. The 5 h data point was used to calculate the turnover frequency of the catalyst.

FIG. 52. Zintl-Klemm ATr₂ and ATrTt phases can catalyze the hydrogenation and semihydrogenation of phenylacetylene with varying activities.

FIG. 53. 4H poly-type of CaGaGe.

FIG. 54. XRD patterns of synthesized 13T (upper trace), synthesized 4H (middle trace), and calculated 4H (lower trace) CaGaGe, with Miller indices labelling the 4H polytype.

FIG. 55. Rietveld refinement of the in-house X-Ray Diffraction (XRD) data, fitting to the published 4H-CaGaGe structure. The hkl values are indicated where the structure is clearly unable to the fit the Okl peaks, where l is odd.

FIG. 56. Observed XRD pattern (upper trace) and superimposed calculated pattern of P3 CaGaGe (lower trace)

FIG. 57. Rietveld refinement of the TOFNeutron powder diffraction pattern. Observed data, calculated pattern, and difference curve, are represented by black dots, a red line, and a blue line, respectively. The hkl tick marks for the 13T and 4H phases are pink and black, respectively. The inset shows larger d-spacing values from a different frame to highlight the 006 and 007 reflections of the 13T structure as well as the 002 reflection of the 4H CaGaGe.

FIG. 58A-FIG. 58B. Atomic position possibilities for gallium and germanium in the P6̅ space group. The ⅓ column represents the (⅓, ⅔, z) position and the 2/3 column represents the (⅔, ⅓, z) position. The middle, planar layer remains unchanged in these initial files. Layer labels (increasing along the c crystallographic axis) increase from top to bottom as visible in the top left column of the table.

FIG. 59A-FIG. 59D. First 65 possibilities for the P3 structure type. Highlighted cells represent where the locations of Ga or Ge are changed within a layer.

FIG. 60A-FIG. 60D. Remaining possibilities for P3 space group. Highlighted cells represent where the locations of Ga or Ge are changed within a layer.

FIG. 61. Flow chart for the refinement procedure with the P6̅ unit cell.

FIG. 62. Final, best structure with the P6̅ space group.

FIG. 63. Rietveld refinement procedure flow chart using the P3 unit cell.

FIG. 64. Refined P3 crystal structure of CaGaGe. Calcium, gallium, and germanium are represented with grey, blue, and purple spheres, respectively. Green text (left) refers to the GaGe layer number. The unique 8^{th}-layer is highlighted by a red font and asterisk. The “A” and “E” on the left column indicates whether “Ga” or “Ge”, respectively, occupy the left (⅓, ⅔, z) positions. An apostrophe next to the A or E denotes that the right (⅔, ⅓, z) atom is above the left (⅓, ⅔, z) atom. The “S” label (right) groups layers having the same atoms stacked on top of each other.

FIG. 65. The catalytic hydrogenation of phenylacetylene to styrene or ethylbenzene using either non-air exposed 13T or 4H CaGaGe and 13T CaGaGe after 5 months air exposure as catalysts. Reaction conditions are 51 bar H₂, 20 h, 2.5 mL solvent (n-BuOH or DMF), 1.82 mmol phenylacetylene, 0.12 mmol CaGaGe (6.2 mol % catalyst).

FIG. 66, Powder XRD patterns of 13T CaGaGe after exposure to air for 1 day (bottom trace), 2 days (second trace from bottom), 6 days (second trace from top), and 2 months (top trace) showing minimal changes.

FIG. 67. Time-dependent conversion of phenylacetylene to styrene or ethylbenzene using non-air exposed 13T-CaGaGe at 1 bar H₂, in 2.5 mL DMF, 0.91 mmol phenylacetylene, 0.081 mmol CaGaGe (8.2 mol % catalyst).

FIG. 68. Superimposed X-ray photoelectron spectra of the Ga 2p_{3/2} and 2p_{1/2} peaks of 4H-CaGaGe (bottom) and 13T-CaGaGe (top). The Ga 2p_{3/2} peak is fit with two peaks: purple corresponds to the non-oxidized shoulder at 1115.5 eV and the red peak corresponds to the more oxidized peak at 1117.3 eV, green is the total calculated fit.

### DETAILED DESCRIPTION

The compositions, methods, and systems described herein may be understood more readily by reference to the following detailed description of specific aspects of the disclosed subject matter and the Examples included therein.

Before the present compositions, methods, and systems are disclosed and described, it is to be understood that the aspects described below are not limited to specific synthetic methods or specific reagents, as such may, of course, vary. It is also to be understood that the terminology used herein is for the purpose of describing particular aspects only and is not intended to be

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### General Definitions

In this specification and in the claims that follow, reference will be made to a number of terms, which shall be defined to have the following meanings.

Throughout the description and claims of this specification the word “comprise” and other forms of the word, such as “comprising” and “comprises,” means including but not limited to, and is not intended to exclude, for example, other additives, components, integers, or steps,

As used in the description and the appended claims, the singular forms “a,” “an,” and “the” include plural referents unless the context clearly dictates otherwise. Thus, for example, reference to “a composition” includes mixtures of two or more such compositions, reference to “an agent” includes mixtures of two or more such agents, reference to “the component” includes mixtures of two or more such components, and the like.

“Optional” or “optionally” means that the subsequently described event or circumstance can or cannot occur, and that the description includes instances where the event or circumstance occurs and instances where it does not.

Ranges can be expressed herein as from “about” one particular value, and/or to “about” another particular value. By “about” is meant within 5% of the value, e.g., within 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

It is understood that throughout this specification the identifiers “first” and “second” are used solely to aid in distinguishing the various components and steps of the disclosed subject matter. The identifiers “first” and “second” are not intended to imply any particular order, amount, preference, or importance to the components or steps modified by these terms.

### Chemical Definitions

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

The term “ion,” as used herein, refers to any molecule, portion of a molecule, cluster of molecules, molecular complex, moiety, or atom that contains a charge (positive, negative, or both at the same time within one molecule, cluster of molecules, molecular complex, or moiety (e.g., Zwitterions)) or that can be made to contain a charge. Methods for producing a charge in a molecule, portion of a molecule, cluster of molecules, molecular complex, moiety, or atom are disclosed herein and can be accomplished by methods known in the art, e.g., protonation, deprotonation, oxidation, reduction, alkylation acetylation, esterification, deesterification, hydrolysis, etc.

The term “anion” is a type of ion and is included within the meaning of the term “ion.” An “anion” is any molecule, portion of a molecule (e.g., Zwitterion), cluster of molecules, molecular complex, moiety, or atom that contains a net negative charge or that can be made to contain a net negative charge. The term “anion precursor” is used herein to specifically refer to a molecule that can be converted to an anion via a chemical reaction (e.g., deprotonation).

The term “cation” is a type of ion and is included within the meaning of the term “ion.” A “cation” is any molecule, portion of a molecule (e.g., Zwitterion), cluster of molecules, molecular complex, moiety, or atom, that contains a net positive charge or that can be made to contain a net positive charge. The term “cation precursor” is used herein to specifically refer to a molecule that can be converted to a cation via a chemical reaction (e.g., protonation or alkylation).

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valencies of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

“Z¹,” “Z²,” “Z³,” and “Z⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” as used herein refers to a non-aromatic hydrocarbon group and includes branched and unbranched, alkyl, alkenyl, or alkynyl groups.

As used herein, the term “alkyl” refers to saturated, straight-chained or branched saturated hydrocarbon moieties. Unless otherwise specified, C₁-C₂₄ (e.g., C₁-C₂₂, C₁-C₁₆, C₁-C₁₀, C₁-C₆, or C₁-C₄) alkyl groups are intended. Examples of alkyl groups include methyl, ethyl, propyl, 1-methyl-ethyl, butyl, 1-methyl-propyl, 2-methyl-propyl, 1,1-dimethyl-ethyl, pentyl, 1-methyl-butyl, 2-methyl-butyl, 3-methyl-butyl, 2,2-dimethyl-propyl, 1-ethyl-propyl, hexyl, 1,1-dimethyl-propyl, 1,2-dimethyl-propyl, 1-methyl-pentyl, 2-methyl-pentyl, 3-methyl-pentyl, 4-methyl-pentyl, 1,1-dimethyl-butyl, 1,2-dimethyl-butyl, 1,3-dimethyl-butyl, 2,2-dimethyl-butyl, 2,3-dimethyl.-butyl, 3,3-dimethyl-butyl, 1-ethyl-butyl, 2-ethyl-butyl, 1,1,2-trimethyl-propyl, 1,2,2-trimethyl-propyl, 1-ethyl-1-methyl-propyl, 1-ethyl-2-methyl-propyl, heptyl, octyl, decyl, dodecyl, tetradecyl, hexadecyl, eicosyl, tetracosyl, and the like. Alkyl substituents may be unsubstituted or substituted with one or more chemical moieties. The alkyl group can be substituted with one or more groups including, but not limited to, hydroxyl, halogen, acyl, alkyl, alkoxy, alkenyl, alkynyl, aryl, heteroaryl, aldehyde, amino, cyano, carboxylic acid, ester, ether, ketone, nitro, phosphonyl, silyl, sulfa-oxo, sulfonyl, sulfone, sulfoxide, or thiol, as described below, provided that the substituents are sterically compatible and the rules of chemical bonding and strain energy are satisfied.

Throughout the specification “alkyl” is generally used to refer to both unsubstituted alkyl groups and substituted alkyl groups; however, substituted alkyl groups are also specifically, referred to herein by identifying the specific substituent(s) on the alkyl group. For example, the term “halogenated alkyl” specifically refers to an alkyl group that is substituted with one or more halides (halogens; e.g., fluorine, chlorine, bromine, or iodine). The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol'” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

As used herein, the term “alkenyl” refers to unsaturated, straight-chained, or branched hydrocarbon moieties containing a double bond. Unless otherwise specified, C₂-C₂₄ (e.g., C₂-C₂₂, C₂-C₂₀, C₂-C₁₆, C₂-C₁₄, C₂-C₁₂, C₂-C₁₀, C₂-C₈, C₂-C₆, or C₂-C₄) alkenyl groups are intended. Alkenyl groups may contain more than one unsaturated bond. Examples include ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2--methyl- -butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl -3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl- 3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, and 1-ethyl-2-methyl-2-propenyl. The term “vinyl” refers to a group having the structure —CH═CH—CH₂; 1-propenyl refers to a group with the structure-CH═CH—CH₃, and 2-propenyl refers to a group with the structure —CH₂—CH═C₂.

Asymmetric structures such as (Z¹Z²)C═C(Z³Z⁴) are intended to include both the E and Z isomers, This can be presumed in structural formulae herein wherein an asymmetric alkene is present, or it can be explicitly indicated by the bond symbol C═C. Alkenyl substituents may be unsubstituted or substituted with one or more chemical moieties, Examples of suitable substituents include, for example, alkyl, alkoxy, alkenyl, alkynyl, aryl, heteroaryl, acyl, aldehyde, amino, cyano, carboxylic acid, ester, ether, halide, hydroxyl, ketone, nitro, phosphonyl, silyl, sulfa-oxo, sulfonyl, sulfone, sulfoxide, or thiol, as described below, provided that the substituents are sterically compatible, and the rules of chemical bonding and strain energy are satisfied.

As used herein, the term “alkynyl” represents straight-chained or branched hydrocarbon moieties containing a triple bond. Unless otherwise specified, C₂-C₂₄ (e.g., C₂-C₂₄, C₂-C₂₀, C₂-C₁₈, C₂-C₁₄, C₂-C₁₂, C₂-C₁₀, C₂-C₈, C₂-C₆, or C₂-C₄) alkynyl groups are intended. Allkynyl groups may contain more than one unsaturated bond, Examples include C₂-C₆-alkynyl, such as ethynyl, 1-propynyl, 2-propynyl (or propargyl), 1-butynyl, 2-butynyl, 3-butynyl, 1-methyl-2-propynyl, 1-pentynyl, 2-pentynyl, 3-pentynyl, 4-pentynyl, 3-methyl-1-butynyl, 1-methyl-2-butynyl, 1 -methyl-3-butynyl, 2-methyl-3-butynyl, 1,1-dimethyl-2-propynyl, 1 -ethyl-2-propynyl, 1-hexynyl, 2-hexynyl, 3-hexynyl, 4-hexynyl, 5-hexynyl, 3-methyl-1-pentynyl, 4-methyl-1-pentynyl, 1-methyl-2-pentynyl, 4-methyl-2-pentynyl, 1-methyl-3-pentynyl, 2-methyl-3-pentynyl, 1-methyl-4-pentynyl, 2-methyl-4-pentynyl, 3-methyl-4-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, and 1-ethyl-1-methyl-2-propynyl. Alkynyl substituents may be unsubstituted or substituted with one or more chemical moieties. Examples of suitable substituents include, for example, alkyl, alkoxy, alkenyl, alkynyl, aryl, heteroaryl, acyl, aldehyde, amino, cyano, carboxylic acid, ester, ether, halide, hydroxyl, ketone, nitro, phosphonyl, silyl, sulfo-oxo, sulfonyl, sulfone, sulfoxide, or thiol, as described below.

As used herein, the term “aryl,” as well as derivative terms such as aryloxy, refers to groups that include a monovalent aromatic carbocyclic group of from 3 to 50 carbon atoms. Aryl groups can include a single ring or multiple condensed rings. In some embodiments, aryl groups include C₆-C₁₀ aryl groups. Examples of aryl groups include, but are not limited to, benzene, phenyl, biphenyl, naphthyl, tetrahydronaphtyl, phenylcyclopropyl, phenoxybenzene, and indanyl. The term “aryl” also includes “heteroaryl,” which is defined as a group that contains an aromatic group that has at least one heteroatom incorporated within the ring of the aromatic group. Examples of heteroatoms include, but are not limited to, nitrogen, oxygen, sulfur, and phosphorus. The term “non-heteroaryl,” which is also included in the term “aryl,” defines a group that contains an aromatic group that does not contain a heteroatom. The aryl substituents may be unsubstituted or substituted with one or more chemical moieties. Examples of suitable substituents include, for example, alkyl, alkoxy, alkenyl, alkynyl, aryl, heteroaryl, acyl, aldehyde, amino, cyano, carboxylic acid, ester, ether, halide, hydroxyl, ketone, nitro, phosphonyl, silyl, sulfa-oxo, sulfonyl, sulfone, sulfoxide, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, acyl, aldehyde, amino, cyano, carboxylic acid, ester, ether, halide, hydroxyl, ketone, nitro, phosphonyl, silyl, sulfa-oxo, sulfonyl, sulfone, sulfoxide, or thiol as described herein.

The term “cycloalkenyl” as used herein is a non-aromatic carbon-based ring composed of at least three carbon atoms and containing at least one double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above, and is included within the meaning of the term “cycloalkenyl,” where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, acyl, aldehyde, amino, cyano, carboxylic acid, ester, ether, halide, hydroxyl, ketone, nitro, phosphonyl, silyl, sulfa-oxo, sulfonyl, sulfone, sulfoxide, or thiol as described herein.

The term “cyclic group” is used herein to refer to either aryl groups, non-aryl groups (i.e., cycloalkyl, heterocycloalkyl, cycloalkenyl, and heterocycloalkenyl groups), or both. Cyclic groups have one or more ring systems that can be substituted or unsubstituted. A cyclic group can contain one or more aryl groups, one or more non-aryl groups, or one or more aryl groups and one or more non-aryl groups.

The term “acyl” as used herein is represented by the formula —C(O)Z¹ where Z¹ can be a hydrogen, hydroxyl, alkoxy, alkyl, alkenyl, alkynyl, aryl, heteroaryl, cycloalkyl, cycloalkenyl, heterocycloalkyl, or heterocycloalkenyl group described above. As used herein, the term “acyl” can be used interchangeably with “carbonyl.” Throughout this specification “C(O)” or “CO” is a shorthand notation for C═O.

The term “acetal” as used herein is represented by the formula (Z¹Z²)C(OZ³)(═OZ⁴), where Z¹, Z², Z³, and Z⁴ can be, independently, a hydrogen, halogen, hydroxyl, alkyl, alkenyl, alkynyl, aryl, heteroaryl, cycloalkyl, cycloalkenyl, heterocycloalkyl, or heterocycloalkenyl group described above.

As used herein, the term “alkoxy” as used herein is an alkyl group bound through a single, terminal ether linkage; that is, an “alkoxy” group can be defined as to a group of the formula Z¹—O—, where Z¹ is unsubstituted or substituted alkyl as defined above. Unless otherwise specified, alkoxy groups wherein Z¹ is a C₁-C₂₄ (e.g., C₁-C₂₂, C₁-C₂₀, C₁-C₁₈, C₁-C₁₆, C₁-C₁₄, C₁- C₁₂, C₁-C₆, or C₁-C₄) alkyl group are intended. Examples include methoxy, ethoxy, propoxy, 1-methyl-ethoxy, butoxy, 1-methyl-propoxy, 2-methyl-propoxy, 1,1-dimethyl-ethoxy, pentoxy, 1-methyl-butyloxy, 2-methyl-butoxy, 3-methyl-butoxy, 2,2-di-methyl-propoxy, 1-ethyl-propoxy, hexoxy, 1,1-dimethyl-propoxy, 1,2-dimethyl-propoxy, -methyl-pentoxy, 2-methyl-pentoxy, 3-methyl-pentoxy, 4-methyl-penoxy, 1,1-dimethyl-butoxy, 1,2-dimethyl-butoxy, 1,3-dimethyl-butoxy, 2,2-dimethyl-butoxy, 2,3-dimethyl-butoxy, 3,3-dimethyl-butoxy, 1-ethyl-butoxy, 2-ethylbutoxy, 1,1,2-trimethyl-propoxy, 1,2,2-trimethyl-propoxy, 1-ethyl-1-methyl-propoxy, and 1-ethyl-2-methyl-propoxy.

The term “aldehyde” as used herein is represented by the formula —C(O)H. Throughout this specification “C(O)” is a shorthand notation for C═O.

The terms “amine” or “amino” as used herein are represented by the formula —NZ¹Z²Z³, where Z¹, Z², and Z³ can each be substitution group as described herein, such as hydrogen, an alkyl, alkenyl, alkynyl, aryl, heteroaryl, cycloalkyl, cycloalkenyl, heterocycloalkyl, or heterocycloalkenyl group described above.

The terms “amide” or “amino” as used herein are represented by the formula—C(O)NZ¹Z², where Z¹ and Z² can each be substitution group as described herein, such as hydrogen, an alkyl, alkenyl, alkynyl, aryl, heteroaryl, cycloalkyl, cycloalkenyl, heterocycloalkyl, or heterocycloalkenyl group described above.

The term “carboxylic acid” as used herein is represented by the formula —C(O)OH. A “carboxy-late” or “carboxyl” group as used herein is represented by the formula —C(O)O⁻.

The term “cyano” as used herein is represented by the formula —CN.

The term “ester” as used herein is represented by the formula —OC(O)Z¹ or —C(O)OZ¹, where Z¹ can be an alkyl, alkenyl, alkynyl, aryl, heteroaryl, cycloalkyl, cycloalkenyl, heterocycloalkyl, or heterocycloalkenyl group described above.

The term “ether” as used herein is represented by the formula Z¹OZ², where Z¹ and Z² can be, independently, an alkyl, alkenyl, alkynyl, aryl, heteroaryl, cycloalkyl, cycloalkenyl, heterocycloalkyl, or heterocycloalkenyl group described above.

The term “ketone” as used herein is represented by the formula Z¹C(O)Z², where Z¹ and Z² can be, independently, an alkyl, alkenyl, alkynyl, aryl, heteroaryl, cycloalkyl, cycloalkenyl, heterocycloalkyl, or heterocycloalkenyl group described above.

The term “halide” or “halogen” or “halo” as used herein refers to fluorine, chlorine, bromine, and iodine.

The term “hydroxyl” as used herein is represented by the formula —OH.

The term “nitro” as used herein is represented by the formula —NO₂.

The term “phosphoryl” is used herein to refer to the phospho-oxo group represented by the formula —P(O)(OZ¹)₂, where Z¹ can be hydrogen, an alkyl, alkenyl, alignyl, aryl, heteroaryl, cycloalkyl, cycloalkenyl, heterocycloalkyl, or heterocycloalkenyl group described above.

The term “silyl” as used herein is represented by the formula —SiZ¹Z²Z³, where and Z³ can be, independently, hydrogen, alkyl, alkoxy, alkenyl, alkynyl, aryl, heteroaryl, cycloalkyl, cycloalkenyl, heterocycloalkyl, or heterocycloalkenyl group described above.

The term “sulfonyl” or “sulfone” is used herein to refer to the sulfa-oxo group represented by the formula —S(O)₂Z¹, where Z¹ can be hydrogen, an alkyl, alkenyl, alkynyl, aryl, heteroaryl, cycloalkyl, cycloalkenyl, heterocycloalkyl, or heterocycloalkenyl group described above.

The term “sulfide” as used herein is comprises the formula —S—.

The term “thiol” as used herein is represented by the formula —SH. “R¹,” “R²,” “R³,” “Rⁿ,” etc., where n is some integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an amine group, an alkyl group, a halide; and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

Unless stated to the contrary, a formula with chemical bonds shown only as solid lines and not as wedges or dashed lines contemplates each possible stereoisomer or mixture of stereoisomer (e.g., each enantiomer, each diastereomer; each meso compound, a racemic mixture, or scalemic mixture).

### Catalysts and Methods of Making and Use Thereof

Disclosed herein are catalysts comprising a layered intermetallic compound comprising:

AM₁₊ₓM′₁₋ₓ

wherein
A is a group 2 metal, a group 3 metal; a lanthanide metal, or a combination thereof;
M is a group 13 metal;
M′ is a group 14 metal or a group 14 metalloid; and
x is from 0 to 1.

In some examples, A can be selected from the group consisting of Ba, Sr, Ca, Lu, Y, La, Ce, Pr, Nd, Sm, Eu, Gd, Tb, Dy, Ho, Er, Tm, Yb; and combinations thereof. In some examples, A can be selected from the group consisting of Ca, Ba, Y, La, Eu, and combinations thereof. In some examples, A can be selected from the group consisting of Ba, Ca, Sr; Y, and combinations thereof. In some examples, A is Ba.

In some examples; A can be selected from the group consisting of Ca, Lu, Y, La, Ce, Pr, Nd, Sm, Eu, Gd, Tb, Dy, Ho, Er, Tm, Yb, and combinations thereof. in some examples, A can be selected from the group consisting of Y, Lu, La, Ce, Pr, Nd, Sm, Eu, Gd, Tb, Dy, Ho, Er, Tm, Yb, and combinations thereof.

In some examples, M can be selected from the group consisting of Al, Ga, In, and combinations thereof. In some examples, M can comprise Ga.

In some examples, M′ can be selected from the group consisting of Si, Ge, Sn, and combinations thereof.

In some examples, x can be 0 or more (e.g., 0,1 or more, 0.2 or more, 0.3 or more, 0.4 or more, 0.5 or more, 0.6 or more, 0.7 or more, 0.8 or more; or 0.9 or more). In some examples, x can be l or less (e.g., 0.9 or less, 0,8 or less, 0.7 or less, 0.6 or less, 0.5 or less. 0.4 or less, 0.3 or less, 0.2 or less, or 0.1 or less). The value of x can range from any of the minimum values described above to any of the maximum values described above. example, x can be from 0 to 1 (e.g., from 0 to 0.5, from 0.5 to 1; from 0 to 0.2, from 0.2 to 0.4, from 0.4 to 0.6, from 0.6 to 0.8, from 0.8 to 1, from 0.1 to 0.9, or from 0.2 to 0.8). In some examples, x is 0 or 1.

In some examples; A is selected from the group consisting of Ba, Sr, Ca, Lu, Y, La, Ce, Pr, Nd, Sm, Eu, Gd, Tb, Dv, Ho, Er, Tm, Yb. and combinations thereof; M is selected from the group consisting of Al, Ga, In, and combinations thereof; and M′ is selected from the group consisting of Si, Ge; Sn, and combinations thereof. In some examples, A is selected from the group consisting of Ba, Sr, Ca, Lu, Y, La, Ce, Pr, Nd, Sm, Eu, Gd, Tb, Dy, Ho, Tm, Yb, and combinations thereof; M is selected from the group consisting of Al, Ga, In, and combinations thereof; M′ is selected from the group consisting of Si, Ge, Sn, and combinations thereof; and x is 0 or 1.

In some examples, A is selected from the group consisting of Ba, Ca, Sr, Y, and combinations thereof; M is selected from the group consisting of Al, Ga, In, and combinations thereof; and M′ is selected from the group consisting of Si, Ge, Sn, and combinations thereof. In some examples; A is selected from the group consisting of Ba, Ca, Sr, Y, and combinations thereof; M is selected from the group consisting of Al, Ga, In, and combinations thereof; M′ is selected from the group consisting of Si, Ge, Sn, and combinations thereof, and x is 0 or 1.

In some examples, A is selected from the group consisting of Ca, Ba, Y, La, Eu, and combinations thereof; M is selected from the group consisting of Al, Ga, In, and combinations thereof, and M′ is selected from the group consisting of Si, Ge, Sn, and combinations thereof. In some examples, A is selected from the group consisting of Ca, Ba, Y, La, Eu, and combinations thereof; M is selected from the group consisting of Al, Ga, In, and combinations thereof; M′ is selected from the group consisting of Si, Ge, Sn, and combinations thereof; and x is 0 or 1.

In some examples, A is selected from the group consisting of Ca, Lu, Y, La, Ce, Pr, Nd, Sm, Eu, Gd, Tb, Dy, Ho, Er, Tin, Yb. and combinations thereof; M is selected from the group consisting of Al, Ga, In, and combinations thereof; and M′ is selected from the group consisting of Si, Ge, Sn, and combinations thereof. In some examples, A is selected from the group consisting of Ca, Lu, Y, La, Ce, Pr, Nd, Sm, Eu, Gd, Tb, Dy, Ho, Er, Tm, Yb, and combinations thereof; M is selected from the group consisting of Al, Ga, In, and combinations thereof; M′ is selected from the group consisting of Si, Ge, Sn, and combinations thereof; and x is 0 or 1.

In some examples, A is selected from the group consisting of Y, Lu, La, Ce, Pr, Nd, Sm Eu, Gd, Tb, Dy, Ho, Er, Tm, Yb, and combinations thereof; M is selected from the group consisting of Al, Ga, In, and combinations thereof; and M′ is selected from the group consisting of Si, Ge, Sn, and combinations thereof. In some examples, A is selected from the group consisting of Y, Lu, La, Ce, Pr, Nd, Sm, Eu, Gd, Tb, Dy, Ho, Er, Tm, Yb, and combinations thereof; M is selected from the group consisting of Al, Ga, In, and combinations thereof; M′ is selected from the group consisting of Si, Ge, Sn, and combinations thereof; and x is 0 or 1.

In some examples, A is Ba; M is selected from the group consisting of Al, Ga, In, and combinations thereof; and M′ is selected from the group consisting of Si, Ge, Sn, and combinations thereof. In some examples, A is Ba; M is selected from the group consisting of Al, Ga, In, and combinations thereof; M′ is selected from the group consisting of Si, Ge, Sn, and combinations thereof; and x is 0 or 1.

In some examples, the catalyst can comprise BaGa₂, BaGaGe, BaGaSn, BalnGe, CaGe₂, CaIn₂, CaGaGe, CaGaSi, SrGa₂, SrAl₂, BaAlSi, SrAlSi, YGa₂, LnGa₂ wherein Ln is a lanthanide metal, or a combination thereof. In some examples, the catalyst can comprise BaGa₂, BaGaGe, BaGaSn, BaInGe, or a combination thereof. In some examples, the catalyst can comprise BaGa₂. In some embodiments, the catalyst is not BaGa₂ or SrGa₂ (i.e., the catalyst is substantially free from BaGa₂ and SrGa₂).

In some examples, the catalyst can comprise BaGaGe, BaGaSn, BalnGe, CaGe₂, CaIn₂, CaGaGe, CaGaSi, SrAl₂, BaAlSi, SrAlSi, YGa₂, LnGa₂ wherein Ln is a lanthanide metal, or a combination thereof.

In some examples, the catalyst can comprise BaGaGe, BaGaSn, BaInGe, CaGe₂, CaGaGe, CaGaSi, BaAlSi, SrAlSi, YGa₂, LnGa₂ wherein Ln is a lanthanide metal, or a combination thereof. In some examples, the catalyst can comprise BaInGe, CaIn₂, CaGaGe, CaGaSi, YGa₂, LaG₂, EuGa₂, or a combination thereof. In some examples, the catalyst can comprise CaGaGe, CaGaSi, YGa₂, or a combination thereof.

In some examples, A is Ba and x is 0 or 1. In some examples, the catalyst can comprise BaGa₂, BaGaGe, BaGaSn, BaInGe, or a combination thereof. In some examples, the catalyst can comprise BaGaGe, BaGaSn, BalnGe, or a combination thereof.

In some examples, x is 1 and M is Ga.

The catalyst can, for example, be substantially free of transition metals.

In some embodiments, the catalyst can comprise CaGaGe. For example, the catalyst can comprise a 13-layer trigonal poly-type of CaGaGe.

Also disclosed herein are catalysts comprising a layered intermetallic compound comprising:

AM₁₊ₓM′₁₋ₓ

wherein
A is a group 2 metal, a group 3 metal, a lanthanide metal, or a combination thereof;
M is a group 13 metal;
M′ is a group 14 metal or a group 14 metalloid; and
x is from 0 to 1;
with the proviso that the catalyst is not BaGa₂ or SrGa₂ (e.g., the catalyst is substantially free from BaGa₂ or SrGa₂).

Also disclosed herein are catalysts comprising a layered intermetallic compound comprising:

AM₁₊ₓM′₁₋ₓ

wherein
A is a group 2 metal, a group 3 metal, a lanthanide metal, or a combination thereof
M is a group 13 metal;
M′ is a group 14 metal or a group 14 metalloid; and
x is from 0 to 1;
with the proviso that when A is Ba or Sr, x is not 1.

In some examples, the catalyst can comprise a layered Zintl phase((e.g., a layered binary-Zintl phase, a layered ternary Zintl phase, or a combination thereof). In some examples, the catalyst can comprise a H₂ adsorbing layered Zintl phase (e.g., a H₂ absorbing layered binary, Zintl phase, a H₂ absorbing layered ternary Zintl phase, or a combination thereof). In some examples, the catalyst can comprise a layered Zintl-Klemm phase comprising group 13 triel or group 14 tetrel networks separated by electropositive cations comprising A. In some examples, the catalyst can comprise a layered Zintl-Klemm phase comprising a triel network, a half-tilled p_{z} orbital, no interlayer bonding in the main group framework, or a combination thereof In some examples, the catalyst can comprise a plurality of particles. The plurality of particles can comprise particles of any shape (e.g., a sphere, a rod, a quadrilateral, an ellipse; a triangle, a polygon, etc.). In some examples, the plurality of particles can have an irregular shape, a regular shape, an isotropic shape, an anisotropic shape, or a combination thereof

In some examples, the catalyst can comprise a plurality of particles having an average particle size. “Average particle size” and “mean particle size” are used interchangeably herein, and generally refer to the statistical mean particle size of the particles in a population of particles. For example, the average particle size for a plurality of particles with a substantially spherical shape can comprise the average diameter of the plurality of particles. For a particle with a substantially spherical shape, the diameter of a particle can refer, for example, to the hydrodynamic diameter. As used herein, the hydrodynamic diameter of a particle can refer to the largest linear distance between two points on the surface of the particle. For an anisotropic particle, the average particle size can refer to, for example, the average maximum dimension of the particle (e.g., the length of a rod shaped particle, the diagonal of a cube shape particle, the bisector of a triangular shaped particle, etc.) For an anisotropic particle, the average particle size can refer to, for example, the hydrodynamic size of the particle. Mean particle size can be measured using methods known in the art, such as evaluation by scanning electron microscopy, transmission electron microscopy, and/or dynamic light scattering.

For example, the catalyst can comprise a plurality of particles having an average particle size of 4 nanometers (nm) or more (e.g., 5 nm or more, 6 nm or more, 7 nm or more, 8 nm or more, 9 am or more, 10 am or more, 15 nm or more, 20 nm or more, 25 nm or more, 30 nm or more, 35 nm or more, 40 nm or more, 45 nm or more, 50 nm or more, 60 nm or more, 70 nm or more, 80 nm or more, 90 nm or more, 100 nm or more, 120 nm or more, 140 nm or more, 160 nm or more, 180 nm or more, 200 nm or more, 220 nm or more, 240 nm or more, 260 nm or more, 280 nm or more, 300 nm or more. 320 nm or more, 340 nm or more, 360 am or more, 380 nm or more, 400 nm or more, 425 nm or more, 450 nm or more, 475 mm or more, 500 nm or more, 550 nm or more, 600 nm or more, 650 nm or more, 700 nm or more, 750 nm or more, 800 nm or more, 900 nm or more, 1 micrometer (him, micron) or more, 1,25 microns or more, 1.5 microns or more. 1.75 microns or more, 2 microns or more, 2.25 microns or more, 2.5 microns or more, 2,75 microns or more, 3 microns or more, 3.5 microns or more, 4 microns or more, 4.5 microns or more, 5 microns or more, 6 microns or more, 7 microns or more, 8 microns or more, 9 microns or more, 10 microns or more, 11 microns or more, 12 microns or more, 13 microns or more, 14 microns or more, 15 microns or more, 20 microns or more, 25 microns or more, 30 microns or more. 35 microns or more, 40 microns or more, or 45 microns or more).

In some examples, the catalyst can comprise a plurality of particles having an average particle size of 50 microns or less (e.g., 45 microns or less, 40 microns or less, 35 microns or less, 30 microns or less, 25 microns or less, 20 microns or less, 15 microns or less, 14 microns or less, 13 microns or less, 12 microns or less, 11 microns or less, 10 microns or less, 9 microns or less, 8 microns or less, 7 microns or less, 6 microns or less, 5 microns or less, 4.5 microns or less, 4 microns or less, 3.5 microns or less, 3 microns or less, 2.75 microns or less, 2.5 microns or less, 2.25 microns or less, 2 microns or less, 1.75 microns or less, 1.5 microns or less, 1.25 microns or less, 1 micron or less, 900 nm or less, 800 nm or less, 750 nm or less, 700 nm or less, 650 nm or less, 600 nm or less, 550 nm or less, 500 nm or less, 475 nm or less, 450 nm or less, 42.5 nm or less, 400 nm or less, 380 nm or less, 360 nm or less, 340 nm or less, 320 nm or less, 300 nm or less, 280 nm or less, 260 nm or less, 240 nm or less, 220 rim or less, 200 nm or less, 180 nm or less, 160 nm or less, 140 nm or less, 120 nm or less, 100 nm or less, 90 nm or less, 80 nm or less, 70 nm or less, 60 nm or less, 50 rim or less, 45 nm or less, 40 nm or less, 35 nm or less, 30 nm or less, 25 nm or less, 20 nm or less, 15 nm or less, 10 nm or less, 9 nm or less, 8 nm or less, 7 nm or less, 6 nm or less, or 5 nm or less).

The average particle size of the plurality of particles of the catalyst can range from any of the minimum values described above to any of the maximum values described above. For example, the catalyst can comprise a plurality of particles having an average particle size of from 4 nm to 50 microns (e.g., from 4 nm to 100 nm, from 100 nm to 1 micron, from 1 micron to 50 microns, from 100 nm to 40 microns, or from 320 nm to 13 microns).

In some examples, the plurality of particles can be substantially monodisperse. “Monodisperse” and “homogeneous size distribution,” as used herein, and generally describe a population of particles where all of the particles are the same or nearly the same size. As used herein, a monodisperse distribution refers to particle distributions in which 80% of the distribution (e.g., 85% of the distribution, 90% of the distribution, or 95% of the distribution) lies within 25% of the median particle size (e.g., within 20% of the median particle size, within 15% of the median particle size, within 10% of the median particle size, or within 5% of the median particle size).

In some examples, the plurality of particles comprises: a first population of particles comprising a first material and having a first average particle size and a first particle shape; and a second population of particles comprising a second material and having a second average particle size and a second particle shape; wherein the first average particle size and the second average particle size are different, the first particle shape and the second particle shape are different, the first material and the second material are different, or a combination thereof.

In some examples, the plurality of particles comprises a mixture of a plurality of populations of particles, wherein each population of particles within the mixture has a different size, shape, composition, or combination thereof. In some examples, the plurality particles within each population of particles are substantially monodisperse.

The catalyst can, for example, have an average surface are of 0.1 meters squared or more per gram of catalyst (m²/g) (e.g., 0.25 m²/g or more, 0.5 m²/g or more, 0.75 m²/g or more, 1 m²/g or more, 1.5 m²/g or more, 2 m²/g or more, 2.5 m²/g or more, 3 m²/g, or more, 3.5 m²/g or more, 4 m²/g or more, 4.5 m²/g or more, 5 m²/g or more, 6 m²/g or more, 7 m²/g or more, 8 m²/g or more, 9 m²/g or more, 10 m²/g or more, 15 m²/g or more, 20 m²/g or more, 25 m²/g or more, 30 m²/g or more, 35 m²/g or more, 40 m²/g or more, 45 m²/g or more, 50 m²/g or more, 60 m²/g or more, 70 m²/g or more, 80 m²/g or more, 90 m²/g or more, 100 m²/g, or more, 125 m²/g or more, 150 m²/g or more, 175 m²/g or more, 200 m²/g or more, 225 m²/g or more, 250 m²/g or more, 275 m²/g or more, 300 m²/g or more, 325 m²/g or more, 350 m²/g or more, or 375 m²/g or more). In some examples, the catalyst can have a surface area of 400 m²/g or less (e.g., 375 m²/g or less, 350 m²/g or less, 325 m²/g or less, 300 m²/g or less, 275 m²/g or less, 250 m²/g or less, 225 m²/g or less, 200 m²/g or less, 175 m²/g or less, 150 m²/g or less, 125 m²/g or less, 100 m²/g or less, 90 m²/g or less, 80 m²/g or less, 70 m²/g or less, 60 m²/g or less, 50 m²/g or less, 45 m²/g or less, 40 m²/g or less, 35 m²/g or less, 30 m²/g or less, 25 m²/g or less, 20 m²/g, or less, 15 m²/g or less, 10 m²/g or less, 9 m²/g or less, 8 m²/g or less, 7 m²/g or less, 6 m²/g or less, 5 m²/g or less, 4,5 m²/g or less, 3 m²/g or less, 3.5 m²/g or less, 2 m²/g, or less, 2.5 m²/g, or less, 2 m²/g or less, 1.5 m²/g or less, 1 m²/g or less, or 0.5 m²/g or less). The average surface area of the catalyst can 20 range from any of the minimum values described above to any of the maximum values described above. For example, the catalyst can have an average surface area of from 0.1 m²/g to 400 m²/g (e.g., from 0.1 m²/g to 200 m²/g, from 200 m²/g to 400 m²/g, from 3 m²/g to 400 m²/g, from 3 m²/g to 230 m²/g, from 1 m²/g to 100 m²/g, from 10 m²/g to 400 m²/g, or from 50 m²/g to 300 m²/g).

The catalysts described herein can be used to catalyze a variety of reactions. In some examples, the catalysts described herein can effectively catalyze a hydrogenation reaction. As used herein a hydrogenation reaction includes semi-hydrogenation and full hydrogenation reactions, In some examples, the catalysts described herein can have a catalytic activity (e.g., a turnover frequency) comparable to the catalytic activity of a commercial Pd based hydrogenation catalyst in a hydrogenation reaction.

For example, the catalysts described herein can effectively catalyze hydrogenation at 1 bar H₂ or more (e.g., 2 bar or more, 3 bar or more, 4 bar or more, 5 bar or more, 6 bar or more, 7 bar or more, 8 bar or more, 9 bar or more, 10 bar or more, 15 bar or more, 20 bar or more, 25 bar or more, 30 bar or more, 35 bar or more, 40 bar or more, 45 bar or more, or 50 bar or more). In some examples, the catalysts described herein can effectively catalyze hydrogenation at 55 bar H₂ or less (e.g., 50 bar or less, 45 bar or less, 40 bar or less, 35 bar or less, 30 bar or less, 25 bar or less, 20 bar or less, 15 bar or less, 10 bar or less, 9 bar or less, 8 bar or less, 7 bar or less, 6 bar or less, 5 bar or less, 4 bar or less, 3 bar or less, or 2 bar or less). The amount of hydrogen provided can range from any of the minimum values described above to any of the maximum values described above. For example, the catalysts described herein can effectively catalyze hydrogenation at 1-55 bar H₂ (e.g., from 1 bar to 25 bar, from 25 bar to 55 bar, from 1 bar to 10 bar, from 10 bar to 20 bar, from 20 bar to 30 bar, from 30 bar to 40 bar, from 40 bar to 55 bar, from 1 bar to 50 bar, or from 1 bar to 40 bar).

In some examples, the catalysts described herein can effectively catalyze hydrogenation at a temperature of 40° C. or more (e.g., 45° C. or more, 50° C. or more, 55° C. or more, 60° C. or more, 65° C. or more, 70° C. or more, 75° C. or more, 80° C. or more, 85° C. or more, 90° C. or more, or 95° C. or more). In some examples, the catalysts described herein can effectively catalyze hydrogenation at a temperature of 100° C. or less (e.g., 95° C. or less, 90° C. or less, 85° C. or less, 80° C. or less, 75° C. or less, 70° C. or less, 65° C. or less, 60° C. or less, 55° C. or less, 50° C. or less, or 45° C. or less). The temperature at which the catalyst effectively catalysts hydrogenation can range from any of the minimum values described above to any of the maximum values described above. For example, the catalysts described herein can effectively catalyze hydrogenation at a temperature of from 40-100° C. (e.g., from 40° C. to 70° C., from 70° C. to 100° C., from 40° C. to 60° C., from 60° C. to 80° C., from 80° C. to 100° C., or from 50° C. to 90° C.).

In some examples, the catalysts described herein can effectively catalyze the hydrogenation of an alkyne. For example, the catalysts described herein can effectively catalyze hydrogenation of an alkyne at 1-55 bar H₂ (e.g., 1-50 bar H₂) and/or at a temperature of 40-100° C.

In some examples, the catalysts described herein can effectively catalyze the hydrogenation of phenylacetylene into styrene and ethylbenzene. For example, the catalyst described herein can effectively catalyze hydrogenation of phenylacetylene into styrene and ethylbenzene at 1-55 bar H₂ (e.g., 1-50 bar H₂) and/or at a temperature of 40-100° C. In sonic examples, the catalysts described herein are effectively air stable, for example such that the effectiveness of the catalyst upon exposure to air for an amount of time of 1 minute or more (e.g., 5 minutes or more, 10 minutes or more, 15 minutes or more, 30 minutes or more, 45 minutes or more, 1 hour or more, 2 hours or more, 3 hours or more, 4 hours or more, 5 hours or more, 6 hours or more, 8 hours or more, 10 hours or more, 12 hours or more, 18 hours or more, 1 day, 2 days or more, 3 days or more, 4 days or more, 5 days or more, 6 days or more, 1 week or more, 2 weeks or more, 3 weeks or more, 1 month or more, 2 months or more, 3 months or more, 4 months or more, 5 months or more, or 6 months or more) decreases by 10% or less (e.g., 9% or less, 8% or less, 7% or less, 6% or less, 5% or less, 4% or less, 3% or less, 2% or less, or 1% or less) relative to the effectiveness of the catalyst in the absence of air exposure.

Also described herein are supported catalysts comprising any of the catalysts described herein disposed on a. support, such as alumina, silica, chromia, zirconia, tungsten, cerium oxide, calcium carbonate, carbon, titanic, metal oxides, metal hydroxides, metal phosphates, or combinations thereof. Also described herein are methods of making the supported catalysts, the methods comprising disposing and/or dispersing the catalyst on the support.

Also described herein are methods of making the catalysts described herein. For example, the methods can comprise heating stoichiometric amounts of A and M and/or M′ followed by annealing, thereby making the catalysts described herein. In some examples, the methods can comprise arc-melting stoichiometric amounts of A and M and/or M′, thereby making the catalysts described herein. In some examples, the methods can further comprise making the catalyst into a plurality of particles, for example by grinding, milling (e.g., air-attrition milling (jet milling) or ball milling), and the like. In some examples, the methods of making the catalysts described herein can include wet chemical methods (e.g., a bottom up method).

Also disclosed herein are methods of hydrogenating a hydrogenation substrate, the methods comprising combining the hydrogenation substrate and hydrogen in the presence of a catalyst, thereby hydrogenating the hydrogenation substrate and forming a mixture, wherein the catalyst comprises any of the catalysts or supported catalysts described herein. As used herein, a “hydrogenation substrate” refers to any compound or element that can be hydrogenated. In some examples, the hydrogenation substrate can comprise an organic substrate including one or more functional groups that can be hydrogenated (e.g., acyl, nitro, alkyne, alkene, aldehyde, ketone, cyano, ester, carboxylic acid, amide, etc.) or an inorganic substrate (e.g., nitrogen). In some examples, the hydrogenation substrate comprises an alkyne. In some examples, the hydrogenation substrate comprises phenylacetylene.

The catalyst can, for example, be provided in an amount of 0.5 mol % or more relative to the amount of the hydrogenation substrate (e.g., 1 mol % or more, 1.5 mol % or more, 2 mol % or more, 2.5 mol % or more, 3 mol % or more, 3.5 mol % or more, 4 mol % or more, 4.5 mol % or more, 5 mol % or more; 5.5 mol % or more, 6 mol % or more, 6.5 mol % or more, 7 mol % or more, 7.5 mol % or more, 8 mol % or more, 8.5 mol % or more, 9 mol % or more, 9.5 mol % or more, 10 mol % or more, 11 mol % or more, 12 mol % or more, 13 mol % or more, 14 mol % or more, 15 mol % or more, 20 mol % or more, 25 mol % or more, 30 mol % or more, 35 mol % or more, 40 mol % or more, or 45 mol % or more). In some examples, the catalyst can be provided.

In an amount of 50 mol % or less relative to the amount of the hydrogenation substrate (e.g., 45 mol % or less, 40 mol % or less, 35 mol % or less, 30 mol % or less, 25 mol % or less, 20 mol % or less, 15 mol % or less, 14 mol % or less, 13 mol % or less, 12 mol % or less, 11 mol % or less, 10 mol % or less, 9.5 mol % or less, 9 mol % or less, 8.5 mol % or less, 8 mol % or less, 7.5 mol % or less, 7 mol % or less, 6.5 mol % or less, 6 mol % or less, 5.5 mol % or less, 5 mol % or less, 4.5 mol % or less, 4 mol % or less, 3.5 mol % or less, 3 mol % or less, 2.5 mol % or less, 2 mol % or less, 1.5 mol % or less, or 1 mol % or less). The amount of catalyst provided relative to the amount of the hydrogenation substrate can range from any of the minimum values described above to any of the maximum values described above. For example; the catalyst can be provided in an amount of from 0.5 mol % to 50 mol % relative to the amount of the hydrogenation substrate (e.g., from 0.5 mol % to 25 mol %, from 25 mol % to 50 mol %, from 0.5 mol % to 40 mol %, from 0.5 mol % to 30 mol %, from 0.5 mol % to 20 mol?, from 0.5 mol % to 10 mol %, from 0.5 mol % to 5 mol %, or from 0.5 mol % to 3 mol %).

The hydrogen can be provided at 1 bar or more (e.g., 2 bar or more, 3 bar or more, 4 bar or more, 5 bar or more, 6 bar or more, 7 bar or more, 8 bar or more, 9 bar or more, 10 bar or more, 15 bar or more, 20 bar or more, 25 bar or more, 30 bar or more, 35 bar or more, 40 bar or more, 45 bar or more, or 50 bar or more). In some examples, the hydrogen can be provided at 55 bar or less (e.g., 50 bar or less, 45 bar or less, 40 bar or less, 35 bar or less, 30 bar or less, 25 bar or less, 20 bar or less. 15 bar or less, 10 bar or less, 9 bar or less, 8 bar or less, 7 bar or less, 6 bar or less, 5 bar or less, 4 bar or less, 3 bar or less, or 2 bar or less). The amount of hydrogen provided can range from any of the minimum values described above to any of the maximum values described above. For example, the hydrogen can be provided at 1-55 bar (e.g., from 1 bar to 25 bar, from 25 bar to 55 bar, from 1 bar to 10 bar, from 10 bar to 20 bar, from 20 bar to 30 bar, from 30 bar to 40 bar, from 40 bar to 55 bar, from 1 bar to 50 bar, or from 1 bar to 40 bar). In some examples, the method can be performed at a temperature of 40° C. or more (e.g., 45° C. or more, 50° C. or more, 55° C. or more, 60° C. or more, 65° C. or more, 70° C. or more, 75° C. or more, 80° C. or more, 85° C. or more, 90° C. or more, or 95° C. or more). In some examples, the methods can be performed at a temperature of 100° C. or less (e.g., 95° C. or less, 90° C. or less, 85° C. or less, 80° C. or less, 75° C. or less, 70° C. or less, 65° C. or less, 60° C. or less, 55° C. or less, 50° C. or less, or 45° C. or less). The temperature at which the method is performed can range from any of the minimum values described above to any of the maximum values described above. For example, the can be performed at a temperature of from 40-100° C. (e.g., from 40° C. to 70° C. from 70° C. to 100° C., from 40° C. to 60° C., from 60° C. to 80° C., from 80° C. to 100° C., or from 50° C. to 90° C.).

In some examples, the method further comprises combining the alkyne and hydrogen source in the presence of the catalyst and a solvent. Examples of suitable solvents include, but are not limited to, tetrahydrofuran (THF), N-methyl-2-pyrrolidone (NMP), dimethylformamide (DMF), dimethyl sulfoxide (DMSO), N,N-dimethylacetamide, dichloromethane (CH₂Co₂), ethylene glycol, ethanol, methanol, propanol, isopropanol, butanol (e.g., n-butanol, n-BuOH), acetonitrile, chloroform, acetone, hexane, heptane, toluene, methyl acetate, ethyl acetate, and combinations thereof. In some examples, the solvent can comprise NMP, n-butanol, or a combination thereof.

The catalyst can, for example, exhibit a turnover frequency of 350 h⁻¹ or more in the hydrogenation (e.g., 400 h⁻¹ or more, 450 h⁻¹ or more, 500 h⁻¹ or more, 550 h⁻¹ or more, 600 h⁻¹ or more, 700 h⁻¹ or more, 800 h⁻¹ or more, 900 h⁻¹ or more, 1000 h⁻¹ or more, 1250 h⁻¹ or more, 1500 h⁻¹ or more, 1750 h⁻¹ or more, 2000 h⁻¹ or more, 2250 h⁻¹ or more, 2500 h⁻¹ or more, 3000 h⁻¹ or more, 3500 h⁻¹ or more, 4000 h⁻¹ or more, 4500 h⁻¹ or more, 5000 h⁻¹ or more, 5500 h⁻¹ or more, 6000 h⁻¹ or more, 6500 h⁻¹ or more, 7000 h⁻¹ or more, 7500 h⁻or more, 8000 h⁻¹ or more, 8500 h⁻¹ or more, 9000 h⁻¹ or more, or 9500 h⁻¹ or more). In some examples, the catalyst can exhibit a turnover frequency of 7400 h⁻¹ or more. In some examples, the catalyst has a catalytic activity in the hydrogenation reaction comparable to the catalytic activity of a commercial Pd based hydrogenation catalyst.

In some examples, the hydrogenation substrate comprises phenylacetylene and hydrogen is provided a 1-55 bar (e.g., 1 -50 bar). In some examples, the hydrogenation substrate comprises phenylacetylene and the method is performed at a temperature of 40-100° C. In some examples, the hydrogenation substrate comprises phenylacetylene, hydrogen is provided at 1-55 bar (e.g., 1-50 bar), and the method is performed at a temperature of 40-100° C. In some examples, the catalyst effectively catalyzes the hydrogenation of phenylacetylene into styrene and ethylbenzene at 1-55 bar (e.g., 1-50 bar) H₂ and at a temperature of 40-100° C.

In some examples, the percent conversion of the hydrogenation substrate is 15% or more within 24 hours at 1 bar H₂ (e.g., 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 95% or more, or 99% or more). In some examples, the percent conversion of the hydrogenation substrate is 80% or more within 48 hours at 1 bar H₂ (e.g., 85% or more, 90% or more, 95% or more, or 99% or more). In some examples, 100% of the hydrogenation substrate is hydrogenated within 2 hours at 51 bar H₂.

In some examples, the hydrogenation substrate is an alkyne and the percent conversion of the alkyne is 15-40% within 24 hours at 1 bar H₂. In some examples, the hydrogenation substrate is an alkyne and the percent conversion of the alkyne is 80% within 48 hours at 1 bar H₂. In some examples, the hydrogenation substrate is an alkyne and 100% of the alkyne is hydrogenated within 2 hours at 51 bar H₂.

In some examples, the methods can further comprise separating the catalyst from the mixture, thereby forming a recycled catalyst. In some examples, the recycled catalyst is used to contact the hydrogenation substrate and hydrogen. The catalyst can, for example, be recycled 4 or more times (e.g., 5 or more, 6 or more, 7 or more, 8 or more, 9 or more, or 10 or more). The catalyst can be separated from the mixture by any suitable method, For example, separating the catalyst form the mixture can comprise decanting, filtering, centrifugation, or a combination thereof.

A number of embodiments of the invention have been described. Nevertheless, it will be understood that various modifications may be made without departing from the spirit and scope of the invention. Accordingly, other embodiments are within the scope of the following claims.

The examples below are intended to further illustrate certain aspects of the systems and methods described herein, and are not intended to limit the scope of the claims.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of measurement conditions, e.g., component concentrations, temperatures, pressures and other measurement ranges and conditions that can be used to optimize the described process.

### Example 1

The discovery of new catalysts has a come a long way from the random screening of solid-state compounds. The most common platform for heterogeneous hydrogenations are supported transition metal nanostructures such as Lindlar's Pb-coated Pd catalysts on CaCO₃ for alkyne semihydrogenation (Lindlar et al. Organic Synthesis 1966, 46, 89), and reduced Fe₃O₄ on calcium alumina silicates or Ru on graphite for NH₃ production. In almost all catalysts, the transition metals are thought to serve as the active sites, enabling the binding of different reactants and intermediates on account of their ability to accommodate various redox states and coordination environments. The support is attributed to a variety of roles including limiting catalyst deactivation from sintering, and, in many instances, influencing the reactivity via spillover (Zaera. Acs Catalysis 2017, 7, 4947). The need to move away from expensive precious metals has resulted in the exploration of methodologies to improve the catalytic activity and selectivity per mass of active metal. Such strategies have included diluting the active metal and tuning the electronic structure via alloying with a second element or achieving higher surface area to mass ratios via nanostructuring. For instance, unsupported PdGa, Pd3Ga₇, and Pd2Ga, and even Al₁₃Fe₄ all exhibit much higher selectivity in acetylene semihydrogenation while maintaining similar activities as Pd on Al₂O₃ (Armbruster et al. Journal of the American Chemical Society 2010, 132, 14745; Armbruster et al. Nature Materials 2012, 11, 690). In addition, computational screening methods to determine the relative binding affinity of different substrates to a catalyst surface have proven to be a powerful method for discovering new heterogeneous catalysts (Studt et al. Science 2008, 320, 1320). For instance, initial predictions of selective alkyne semi-hydrogenation with NiGa and NiSn (Studt et al. Science 2008, 320, 1320), led to the experimental observation of this catalysis in Ni₃Ga and Ni₃Sn₂ nanocrystals (Liu et al. Advanced. Materials 2016, 28, 4747). A final approach that has emerged is to identify solids with particular electronic structures that are thought to be correlated with high catalytic activity. One example of this are the solid-state electrides that have low work functions and can readily dissociate H₂ (Zaera. Acs Catalysis 2017, 7, 4947). Recently, LaCu_{0.67}Si_{1.33} was reported to be a highly stable, solid-state electride with excellent catalytic activity for nitroarene hydrogenation (Ye et al. Journal of the American Chemical Society 2017, 139, 17089). LaRuSi, and LaCoSi have been reported to be effective for ammonia synthesis (Gong et al. Nature Catalysis 2018, 1, 178; Wu et al. Angewandte Chemie-International Edition 2019, 58, 825). Still, there remains a need of developing transition-metal free heterogeneous hydrogenation catalysts.

The discovery and screening of unexpected families of heterogeneous hydrogenation catalysts described herein was based off of the propensity of these materials to react with and absorb Hz into their lattice under moderate conditions (<200° C., 50 bar H₂) (Haussermann et al. In Zintl Phases: Principles and Recent Developments; Fassler, T. F., Ed, 2011; Vol. 139, p 143). One of the key steps in most hydrogenation mechanisms is the ability for a solid to dissociatively adsorb H₂ onto the surface of its lattice. A solid that can break apart H₂ and absorb it deep within its lattice under relatively mild conditions is likely to have a low barrier for surface adsorption. Whether the internally absorbed hydrogen plays a role in catalysis depends on the material and the catalytic reaction. Pd metal is one of the best hydrogen absorbing metals, being able to incorporate 0.7 eq of H into its crystal structure (Worsham et al. Journal of Physics and Chemistry of Solids 1957, 3; 303), for which this subsurface hydride is found to result in alkyne over-hydrogenation to the alkane (Garcia-Iota et al. Journal of Catalysis 2010, 273, 92; Pradier et al. Journal of Molecular Catalysis 1994, 89, 211; Teschner et al. Science 2008, 320, 86; Teschner et al. Journal of Catalysis 2006, 242, 26). In contrast, LaCu_{0.67}Si_{1.33} also forms a hydride (LaCuo_{0.67}Si_{1.33}H_{0.3}) for which catalytic activity is greatly suppressed (Ye et al. Journal of the American Chemical Society 2017, 139, 17089).

Inexpensive, transition metal-free intermetailic compounds have received almost no attention as heterogeneous catalysts. Herein, BaGa₂, a transition metal-free, layered Zintl-Klemm compound comprised of honeycomb sheets of Ga⁻ anions separated by Ba⁺ and which can react with H₂ under modest conditions to form a layered poly anionic hydride BaGa₂H₂, effectively catalyzes the hydrogenation of phenylacetylene into styrene and ethylbenzene under modest conditions (1-50 bar 40-100° C.), is shown to be a highly efficient catalyst for alkyne hydrogenation. For example, BaGa₂ effectively catalyzes the hydrogenation of phenylacetylene into styrene and ethylbenzene under modest conditions (1-50 bar H₂, 40-100° C.). Using phenylacetylene as a substrate, turnover frequencies (TOFs)>350 h⁻¹ were observed with very mild conditions (40° C., 1 bar H₂, 4 mol % catalyst), as well as much higher TOFs (>7400 h⁻¹) were observed at elevated H₂ pressures. Remarkably, the catalytic activity of BaGa₂ (TOF up to 7400 h⁻¹), is in the same order of magnitude as commercial Pd-based catalysts. In contrast, the hydrogenated form BaGa₂H₂ showed no appreciable catalytic activity thereby indicating that hydrogen inside the Zintl phase is kinetically trapped and does not participate in catalysis and that the unsaturated Ga⁻ framework is necessary for phenylacetylene and styrene adsorption. Despite the high activity, the Zintl phase surface is prone to oxidation in H₂O, suppressing catalytic activity. The phenylacetylene semihydrogenation and hydrogenation reactions can be modeled to first order kinetics with respect the organic substrate. Overall these results indicate that catalytic activity can be achieved in main group Zintl phases. These findings open up future explorations of utilizing and optimizing the long term stability of transition metal-free intermetallic hydrogen absorbing compounds for hydrogen based catalysis.

### Experimental

Sample Preparation. The synthesis of BaGa₂ was accomplished with greater than 90% phase purity via a sealed quartz tube reaction in an alumina crucible by slowly heating stoichiometric quantities of barium and gallium to 900° C. and holding at that temperature for 12 hours. The reaction was then cooled over 12 hours to 100 ° C., then reheated to 800° C., and held at 800° C. for 24 hours. The impurity phase, BaGa₄, was also synthesized by heating up stoichiometric barium and gallium over 12 hours to 900° C., held for 12 hours, and then cooled to room temperature over 12 hours. BaGa₂H₂ was synthesized by annealing BaGa₂ at 50 bar H₂ and 175° C. for 5 days.

General Procedure for the Catalytic Hydrogenation Reactions. For atmospheric pressure experiments, BaGa₂ was ground in a mortar and pestle, massed out, and added to a 10 mL round bottom flask, followed by the addition of the dry, degassed solvent and phenylacetylene. In a typical reaction, 0.9 mmol of substrate and 10 mg of catalyst were mixed in 2.4 mL of solvent. The material was then removed from the glovebox, and a balloon filled with hydrogen gas was added to the flask (FIG. 18). The reaction progress as a function of time was monitored via HPLC with an external calibration to monitor the progress from phenylacetylene to styrene to ethylbenzene, For high pressure experiments, the catalyst, solvent, and substrate were added to a 20 mL vial in a Parr bomb. Once the Parr bomb was brought out of the glovebox it was pressurized with hydrogen gas and then brought up to temperature on a hot plate. To calculate the surface area normalized turnover frequencies, Equations 1, 2, and 3 were used:

$$TOF = {n}_{0} C / {tn}_{cat} \qquad ( 1 )$$

$${n}_{cat} = {m}_{cat} {N}_{Ga sites} / {N}_{A} \qquad ( 2 )$$

$${N}_{Ga sites} = \frac{{S}_{A cat}}{V \times ρ} \times \frac{2 Ga atoms}{{S}_{A Ga}} \qquad ( 3 )$$

where n₀ is the initial moles of substrate, C is the conversion of the substrate at reaction time t, n_{cat} is the moles of Ga atoms exposed on the surface, m_{cat} is the mass of the catalyst, N_{Ga sites} is the amount of exposed Ga atoms per gram of catalyst, N_{A} is Avogadro's constant, S_{A cat} is the surface area of the catalyst, V is the volume of the catalyst, ρ is the density of the catalyst, and S_{AGa} on is the surface area of the gallium atoms.

### Results and Discussion

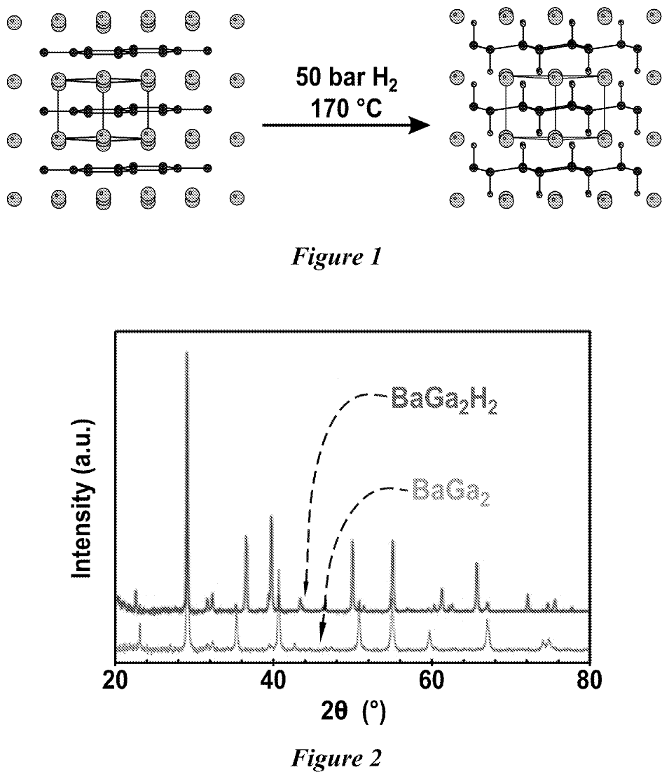

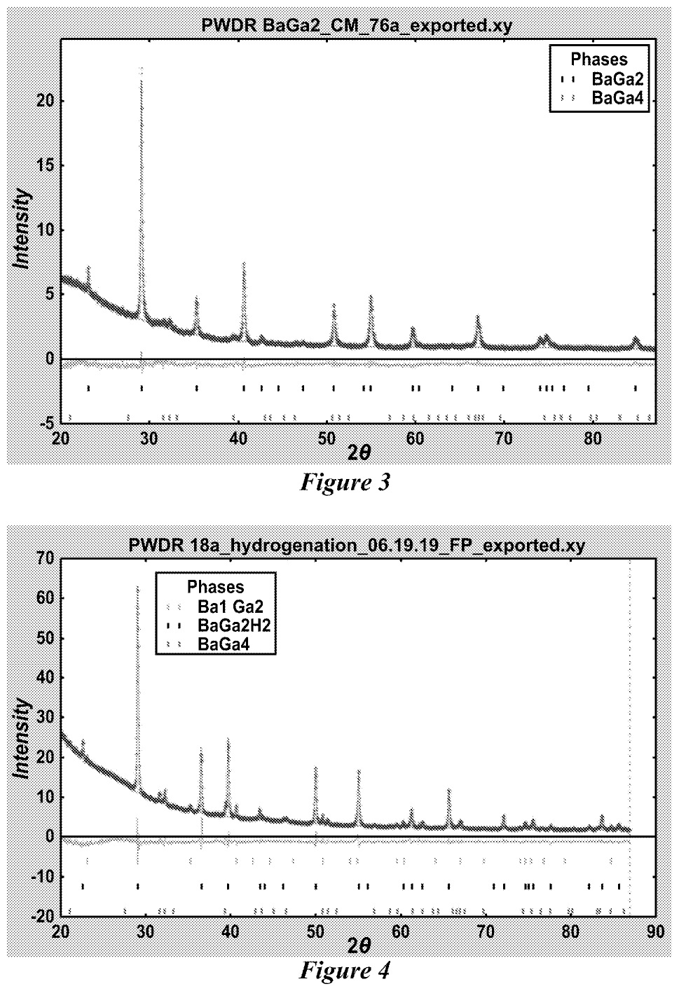

The BaGa₂ crystal structure comprises planar honey-comb networks of Ga⁻ anions separated by Ba²⁺ cations (FIG. 1). This crystal structure is isoelectronic to MgB₂ or graphite. Thus, the electron configuration suggests that the Ga networks have appreciable π-bonding. In contrast to the first-row elements, Ga—Ga π-bonding is much weaker. Consequently, under relatively mild conditions (50 bar H₂ and 170° C.) BaGa₂ readily reacts with H₂ to form BaGa₂H₂ (Bjorling et al, Journal of the American Chemical Society 2006, 128, 817). The polyanionic gallium hydride layer retains the honeycomb configuration, but is now puckered on account of Ga's sp³-hybridization (FIG. 1). BaGa₂ was synthesized by heating stoichiometric amounts of barium and gallium in an alumina crucible in a sealed quartz tube for 12 hr at 900° C., followed by a 24 hr anneal at 800° C. FIG. 2 shows the powder X-ray diffraction (XRD) pattern of the BaGa₂ product. Rietveld analysis confirmed that the optimized synthesis of BaGa₂ was >97% phase pure and crystallized into a P6/mmm space group with a=4.4320(3) Å and c=5.0731(2) Å (FIG. 3, Table 1).

TABLE 1
Rietveld analysis of BaGa₂.

| Empirical Formula | BaGa₂ |
|---|---|
| Fw (g/mol) | 276.77 |
| Space group | P6/mmm |
| a (Å) | 4.4320(3) |
| c (Å) | 5.0731(2) |
| V (Å³) | 86.301 |
| Z | 3 |
| T (K) | 295 |
| λ (Å) | 1.5406 |
| ρ (g cm⁻³) | 5.325 |
| Pattern range (2θ, °) | 15-87 |
| Step size (2θ, °) | 0.015342 |
| Step scan time (s) | 2.0 |
| No. of contributing reflns | 4368 |
| R_{wp} | 0.0492 |
| Rₚ | 0.0377 |
| R(F²) | 0.0565 |
| BaGa₂ Phase Fraction | 0.9773 |
| BaGa₄ Phase Fraction | 0.0276 |

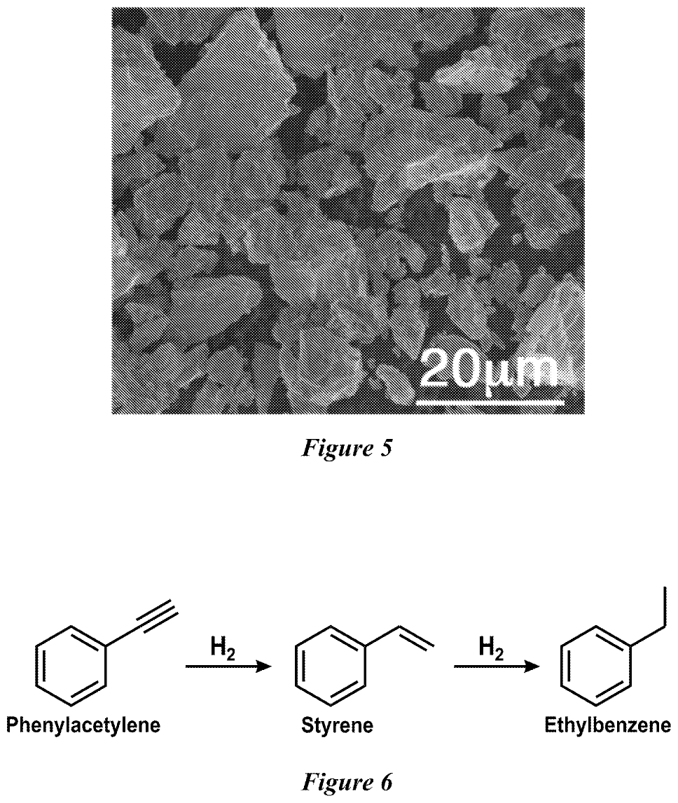

Due to the peritectic nature of BaGa₂ in the Ba-Ga phase diagram (Okamoto. Journal of Phase Equilibria and Diffusion 2015, 36, 518), trace quantities of BaGa₄ were usually found under a wide range of synthetic conditions. Therefore, pure BaGa₄ as synthesized to compare its catalytic activity as a potential catalytic impurity (FIG. 4). Also, it was confirmed that the majority of the BaGa₂ phase transformed into BaGa₂H₂ upon reacting it for 7 days at 170° C. under 50 bar ft. FIG. 2 also shows the XRI) pattern of the hydrogenated sample, showing a majority BaGa₂H₂ phase. Rietveld analysis (FIG. 4, Table 2) confirmed that the majority of the sample (84.7 wt %) transformed into a P3ml space group with a=4.530(8) Å and c=4.910(9) Å, with 11.6% unreacted BaGa₂ and ˜3.7 wt % is BaGa₄. Scanning Electron Microscope (SEM) images of the ground BaGa₂ powders shows particles with lengths and widths with dimensions ranging from 320 nm to 13 μm (FIG. 5).

TABLE 2
Rietveld analysis of BaGa₂H₂.

| Empirical Formula | BaGa₂H₂ |
|---|---|
| Fw (g/mol) | 278.79 |
| Space group | P-3m1 |
| a (Å) | 4.530(8) |
| c (Å) | 4.910(9) |
| V | 86.385 |
| Z | |
| T | 295 |
| λ | 1.5406 |
| ρ (g cm⁻³) | 5.302 |
| Pattern range (2θ, °) | 15-87 |
| Step size (2θ, °) | 0.020 |
| Step scan time (s) | 1.00 |
| No. of contributing reflns | 3397 |
| R_{wp} | 0.0481 |
| Rₚ | 0.0355 |
| R(F²) | 0.0814 |
| BaGa₂ Phase Fraction | 0.116 |
| BaGa₄ Phase Fraction | 0.0365 |
| BaGa₂H₂ Phase Fraction | 0.847 |

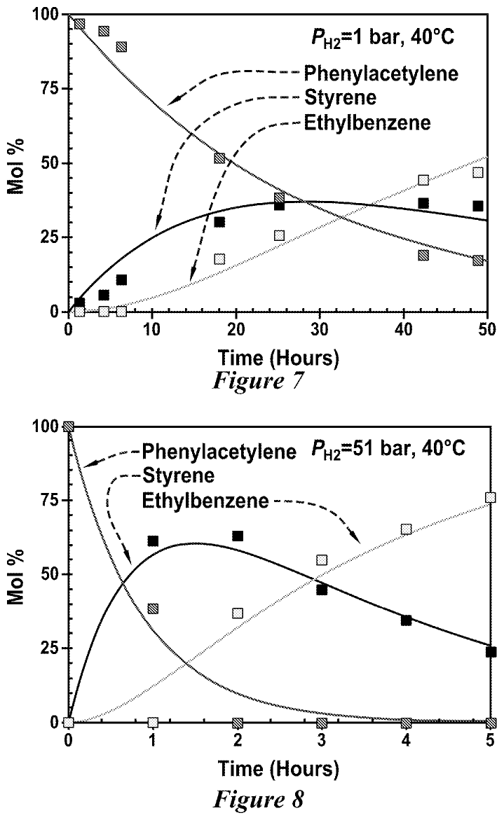

Preliminary hydrogenation experiments were conducted using phenylacetylene as a substrate to evaluate the catalytic semi-hydrogenation to styrene and full hydrogenation to ethylbenzene (FIG. 6). Using 1 bar H₂ and 0.5-3 mol % BaGa₂ it was found that the dried, freshly-distilled, and degassed solvents DMF (40° C.) and n-butanol (70° C.) had the highest percent conversion of phenylacetylene to styrene and ethylbenzene (15-40% within 24 hrs). Other solvents such as THF and MeOH had much less conversion of phenylacetylene (5-10%) after 24 hrs. As shown in FIG. 7, 80% conversion of phenyl acetylene to styrene and ethylbenzene was observed after 48 hr in DMF, 1 bar H₂, with 4.7 mol % BaGa₂. Longer reaction times did not lead to 100% conversion, which is a consequence of solvent decomposition and catalyst poisoning. No other products other than ethylbenzene or styrene were detected via 1H NMR, or HPLC. Additionally, BaGa₄ and BaGa₂H₂ controls showed minimal catalytic activity, even at 51 bar H₂ (<10% conversion after 20 hr). BaGa₄ is a binary analog of the ThCr₂Si₂ structure-type, and comprises layers of 2D tetrahedrally coordinated Ga₄²⁻ sheets. The lack of activity using BaGa₄ partially implies that the n-bonding honeycomb network and electronic structure of BaGa₂ can be important for this transformation. It was hypothesized that when the Ga atoms are coordinatively saturated upon forming BaGa₂H₂, catalytic activity is disrupted via blocking substrate adsorption upon elimination of the π-network. This loss of activity upon hydrogenating the catalyst is consistent with the aforementioned loss of catalytic activity in LaCu_{0.67}Si_{1.33}H_{0.3} (Ye et al. Journal of the American Chemical Society 2017, 139, 17089). Finally, once the BaGa₂ catalyst is removed, the activity is eliminated indicating the absence of a trace homogeneous catalytic impurity being responsible for catalysis.

The TOF was calculated by measuring the surface area per gram of the BaGa₂ catalyst, determined via counting the sizes of >300 particles using SEM analysis, along with the conversion vs. time. The average surface area of the ground particles was estimated to be ˜3.4 m² g⁻¹. Given that BaGa₂ has 2 Ga atoms per surface area of 17.0 Å² in a single unit cell, the turnover frequency at 40° C. was estimated to be 370 ⁻¹. This turnover frequency makes BaGa₂ the most active Pd-free heterogeneous catalyst for phenylacetylene hydrogenation and is within 1-2 orders of magnitude of the most active Pd-catalysts under similar conditions (Table 3).

TABLE 3
Phenylacetylene to Styrene Turnover Frequency, normalized to the relative
number of surface atoms of Ga (for BaGa₂), Pd, Au, or Ni (for Ni₃Ga)

| Catalyst/Support | Condition | TOF | | Reference |
|---|---|---|---|---|
| BaGa₂/None | 51 bar H₂, | 7400 | h⁻¹ | — |
| | 40° C., DMF | | | |
| BaGa₂/None | 1 bar H₂, | 370 | h⁻¹ | — |
| | 40° C., DMF | | | |
| Ni₃Ga/None | 5 bar H₂, | 5.16 × 10⁻³ | h⁻¹ | Liu et al.Adv. Mater. |
| | 40° C., Hexanes | | | 2016, 28, 4747-4754 |
| Pd/Carbon | H₂ flow (30 mL/min), | 3600 | h⁻¹ | Dominguez-Dominguez et |
| Nanotubes | 50° C., Methanol | | | al. J. Phys. Chem. C. |
| | | | | 2008, 112, 10, 3827-3834 |
| Au/Graphene | H₂ flow (0.2 mL/min), | 360 | h⁻¹ | Shao et al. ACS Catal. |
| Oxide | 60° C., Ethanol | | | 2014, 4, 7, 2369-2373 |
| PdZn/ZnO | 6 bar H₂, | 15840 | h⁻¹ | Yoshida et al. RSC. Adv., |
| | 0° C., Toluene | | | 2014, 47, 24922-24928 |
| Pd/(mesoporous | 1 bar H₂, | 47304 | h⁻¹ | Yang et al. Chemistry Select |
| SiO₂ + Fe₃O₄) | 40° C., Ethanol | | | 2016, 1, 5599-5606 |
| Lindlar's Catalyst | Continuous flow 1 bar | 1866 | h⁻¹ | Kuwahara et al. ACS Catal. |
| | H₂, Methanol, | | | 2019, 9, 1993-2006 |
| | 30° C., 1,4- dioxane | | | |
| Pd/(Polyethylene | Continuous flow 1 bar | 6751 | h⁻¹ | Kuwahara et al. ACS Catal. |
| imine + SiO₂ | H₂, 30° C., | | | 2019, 9, 1993-2006 |
| | Methanol, 1,4- dioxane | | | |
| Pd/(mesoporous | 1 bar of H₂, | 1840 | h⁻¹ | Guo et al. ACS Catal. |
| SiO₂ FDU-12) | 25° C., Ethanol | | | 2018, 8(7), 6476-6485 |

Additional hydrogenation experiments were performed under elevated pressure with the two solvents that displayed best activity, DMF and n-butanol. In DMF, phenylacetylene was completely converted to styrene and ethylbenzene within 2 hours at 40° C. using 51 bar H₂and 5.5 mol % catalyst (FIG. 8). After 15 hours the styrene was completely hydrogenated to ethylbenzene. The maximum turnover frequency for this catalyst was ˜7400 h^{—1}. The kinetic traces at both low and high H₂ pressure show excellent fits to consecutive reactions that both exhibit first order reaction kinetics to their respective substrates (FIG. 7, FIG. 8). At 1 bar H₂, the first order rate constant of phenylacetylene to styrene fits to k₁=0.035(4) h⁻¹ and, the first order rate constant of styrene to ethylbenzene fits to k₂=0.035(7) h⁻¹ (Table 4). At 51 bar H₂, the rate constants are still first order and much faster, with k₁=1.16(19) h⁻¹ and k₂=0.337(23) h⁻¹ (Table 4). These differences with respect to H₂ pressure indicate the phenylacetylene to styrene conversion reaction approaches first order with respect to H₂, and the styrene to ethylbenzene conversion reaction approaches ½ order with respect to H₂. For comparison, in the conversion of ethylene to ethane, the order of the rate law with respect to H₂ typically varies from order to order depending on the catalyst and conditions. In addition, in the conversion of acetylene to ethylene, most metals show first order kinetics with respect to H₂ pressure, comparable to the observations herein with phenylacetylene hydrogenation.

TABLE 4
Rate constants from first order kinetic fits in FIG. 7 and FIG. 8.

| | 1 bar | 51 bar | |
|---|---|---|---|
| k₁ | 0.035(4) h⁻¹ | 1.16(19) | h⁻¹ |
| k₂ | 0.035(7) h⁻¹ | 0.337(23) | h⁻¹ |

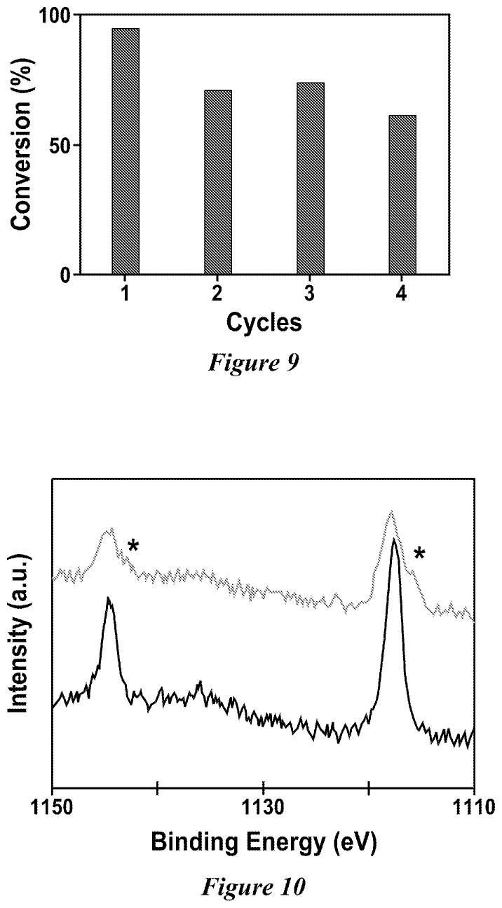

In n-butanol the conversion rate was slower than DMF, giving 95% phenylacetylene conversion at 100° C. after 20 hours (FIG. 9). FIG. 9 also demonstrates the reusability of the catalyst. The same catalyst retains greater than 50% conversion after four 20 hr cycles. The decreased activity over time can be attributed to formation of a surface oxide. FIG. 10 shows a zoom in of the Ga 2p region of the X-ray Photoelectron spectra (XPS) of two different BaGa₂ catalysts: one that has been exposed to air and one that has only been exposed to an Ar-filled glove box. After the BaGa₂ catalyst is exposed to air, the 2p_{3/2} and 2p_{1/2} peak appear at 1117.90 and 1144.55 eV, indicative of an oxidized Ga³⁺ (Priyantha et al. Journal of Crystal Growth 2011, 323, 103). The sample that has been solely handled in an Ar-filled glovebox has a surface that is still partially oxidized, on account of the presence of peaks at the same energies. However, it shows the existence of a surface species that has a lower binding energy than Ga metal, which would be expected for BaGa₂, due to the presence of the 2p_{3/2} and 2p_{1/2} shoulder at 1115.70 and 1142.69 eV. Further confirming that surface oxidation can inhibit catalysis, exposure of the catalyst to either undistilled solvents or air/H₂O immediately eliminated all catalytic activity. Annealing air-exposed BaGa₂ catalyst at 600-800° C. can restore the catalytic activity. It is not unexpected that this Ga containing compound is prone to catalytic deactivation via oxidation, as even catalytic activity in PdGa and Pd₃Ga₇ is significantly decreased upon oxidation (Kovnir et al. Journal of Catalysis 2009, 264, 93). It is possible that the surface oxide can be removed in nonaqueous solution-conditions by exploiting the amphoteric nature of the group 13 metal.

BaGa₂, a transition-metal free intermetallic Zintl phase, has a high catalytic activity for alkyne hydrogenation. In the conversion of phenylacetylene to styrene, the turnover frequencies are among the highest for non-Pd containing catalysts. The exploration of these materials can investigate long-term oxidative stability, substrate selectivity, and a mechanistic understanding. BaGa₂ is just one member of a large family of hydrogen-absorbing layered honeycomb Zintl phases.

### Example 2

The heterogeneous catalysis of petroleum feedstocks into functionally useful derivatives using inexpensive and earth abundant elements remains an important goal. The semihydrogenation of C≡C triple bonds into C═C double bonds and full hydrogenation into C—C single bonds is an industrially significant heterogeneously catalyzed reaction, yet it is performed almost exclusively using palladium catalyst.

Described herein are a family of transition-metal free alkyne semihydrogenation catalysts and hydrogenation catalysts using layered intermetallic phases having the general stoichiometry AM₁₊ₓM′₁₋ₓ, wherein: A is a group 2 metal, a group 3 metal, a lanthanide metal, or a combination thereof; M is a group 13 metal; M′ is a group 14 metal or a group 14 metalloid; and x is from 0 to 1.

For example, BaGa₂, a Zintl-Klemm compound comprising honeycomb sheets of Ga⁻ anions separated by Ba²⁺, which can react with H₂ under modest conditions to form a layered polyanionic hydride BaGa₂H₂, was shown to effectively catalyze the hydrogenation of phenylacetylene into styrene and ethylbenzene under modest conditions (1-50 bar H₂, 40-100° C.). The catalytic activity of BaGa₂ (TOF up to 7400 h⁻¹) is in the same order of magnitude as commercial Pd-based catalysts. In contrast, the hydrogenated form BaGa₂H₂ shows negligible catalytic activity, indicating that the unsaturated Ga⁻ framework is involved in the phenylacetylene and styrene adsorption.

For example, CaGaGe effectively catalyzes the hydrogenation of phenylacetylene into styrene and ethylbenzene under modest conditions (1-50 bar H₂, 40-100° C.). Remarkably, the catalytic activity of CaGaGe is even better than BaGa₂ (TOE up to 7400 h⁻¹), which is in the same order of magnitude as commercial Pd-based catalysts. These findings open up a new family of transition metal-free intermetallic hydrogen absorbing compounds for hydrogen based transformations.

### Example 3

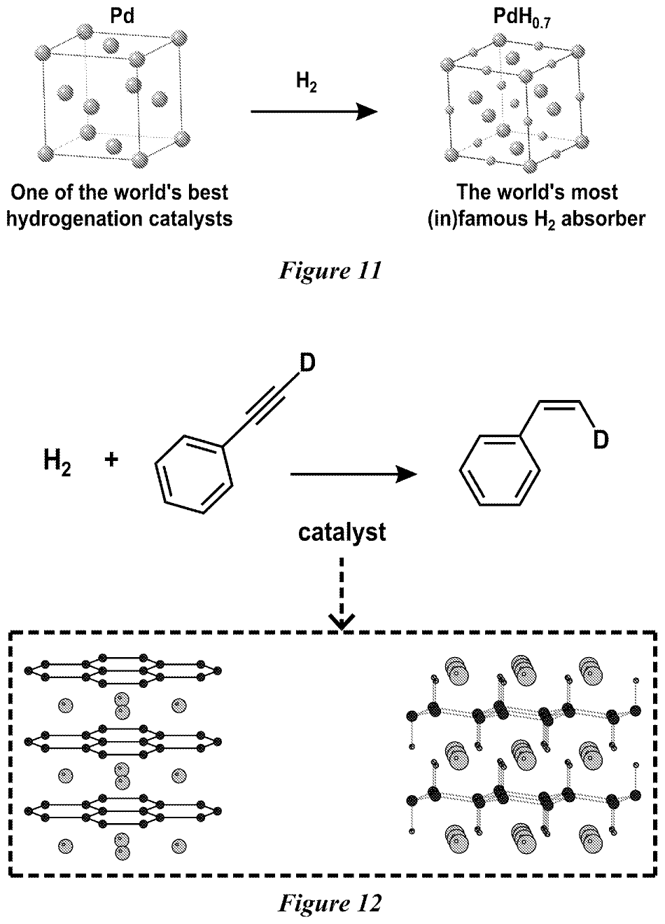

It is estimated that between 10-20% of all industrial catalytic processes performed today are hydrogenations in some form, These are typically performed heterogeneously using solid-state compounds that contain Pd, Pt, or other rare and expensive precious metals, because of their affinity for hydrogen (FIG. 11). Alkyne semi-hydrogenation is an industrial example with an unwanted over-hydrogenation product ethylbenzene (FIG. 6) usually with use of Lindlar's catalyst Pd/Pb on CaCO₃.

However, there are many classes of precious-metal free compounds that readily cleave dihydrogen and absorb it into their lattices under moderate pressures (1-50 Bar) and temperatures (50-200° C.). For instance, layered BaGa₂ forms layered polyanionic hydrides with covalent Ga—H bonds.

Described herein are hydrogen absorbing Zintl phases which can be used as heterogeneous catalysts, for example in hydrogenation reactions (FIG. 12). For example, it is shown herein that the Zintl-Klemm compound BaGa₂ can effectively catalyze the semi-hydrogenation of phenylacetylene into styrene at modest temperatures (40-70° C.). The catalytic activity of BaGa₂ is on par with the commercial Undies catalyst

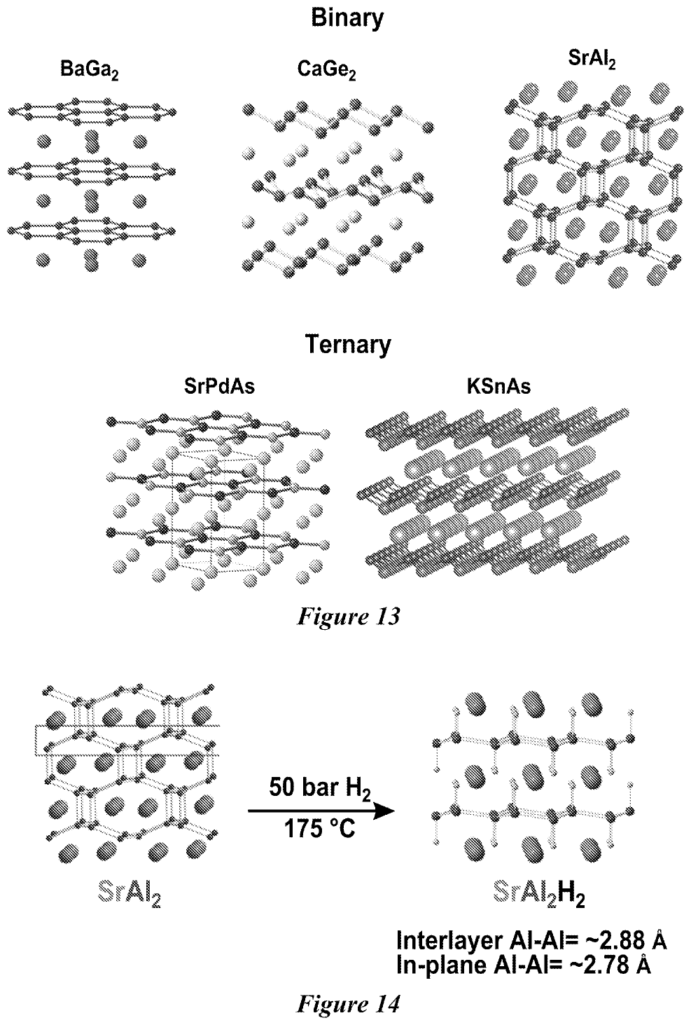

Layered Zintl phases are intermetallics having a general stoichiometry of AE₂, wherein A comprises a Group 1, Group 2, or Lanthanide metal and E comprises a main group element. Large electronegativity differences between A and E can create the layered Zintl phases. Many layered phases occur with AE₂ stoichiometry. Examples of binary and ternary layered Zintl phases are shown in FIG. 13.

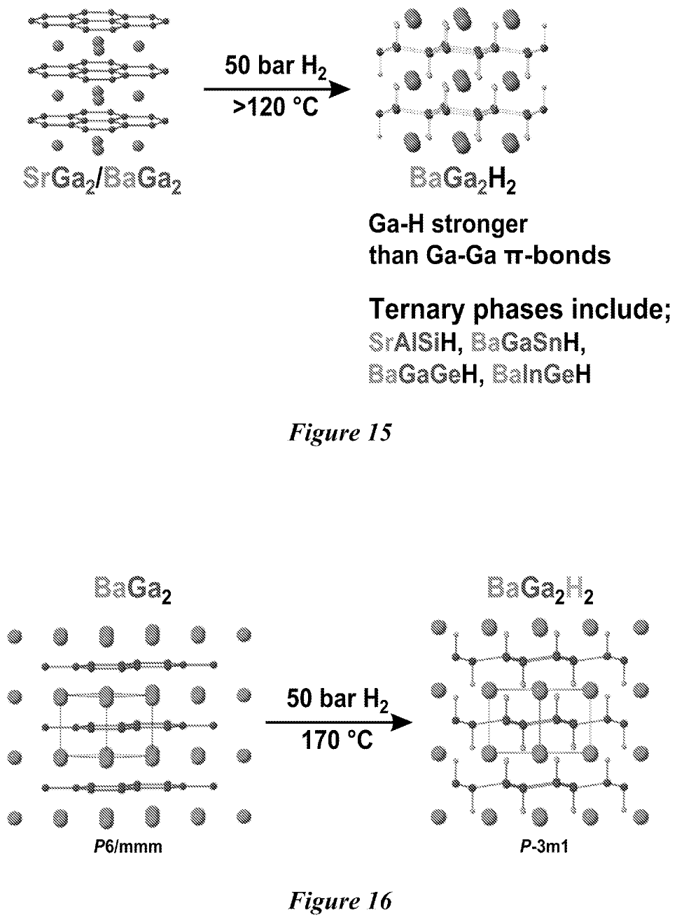

Some Zintl phases can absorb hydrogen. An example of a hydrogen absorbing Zintl phases is SrAl₂, which becomes SrAl₂H₂ upon absorption of hydrogen (FIG. 14) (Gingl et at J Alloys Compd, 2000, 306, 127-132). Another example of a hydrogen absorbing Zintl phase is SrGa₂/BaGa₂ (FIG. 15) (Haussermann et al. JACS, 2006, 128(3), 817-824; Auer et al. Inorganic Chemistry 2017, 56 (3), 1061-1071; Wenderoth et al. Inorganic Chemistry 2013, 52 (18) , 10525-10531; Wenderoth et al. Zeitschrift für anorganische und allgemeine Chemie 2012, 638 (10) , 1604-1604).

Weak π-bonding in the main group layer of BaGa₂ allows for polyanionic hydrogen incorporation into the bulk lattice (FIG. 16). BaGa₂ was synthesized and characterized using X-Ray diffraction (FIG. 2). Scanning electron microscopy was used to characterize the BaGa₂ particles (FIG. 3), for example to determine that the average surface area of the BaGa₂ particles was 13.428 μm².

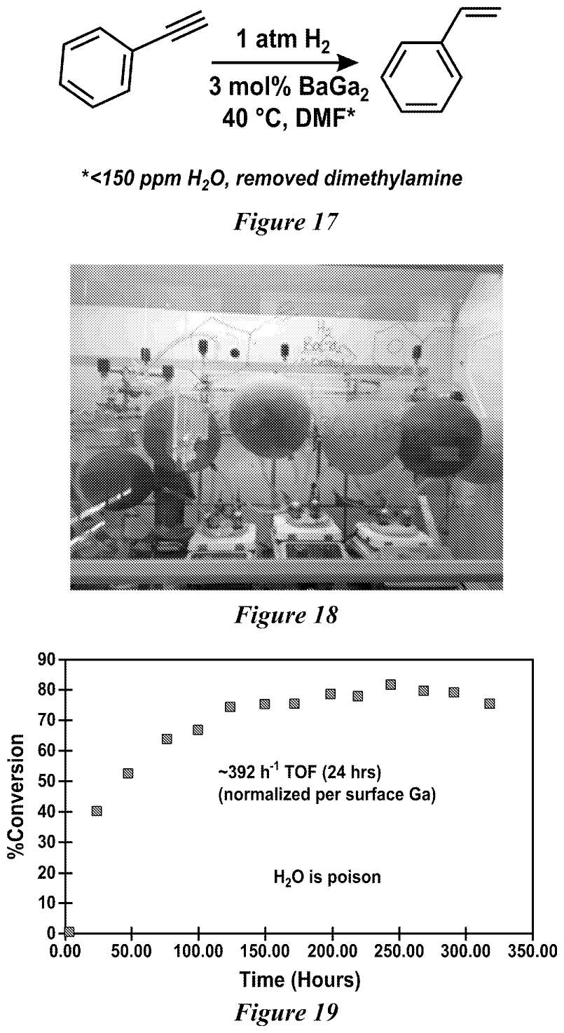

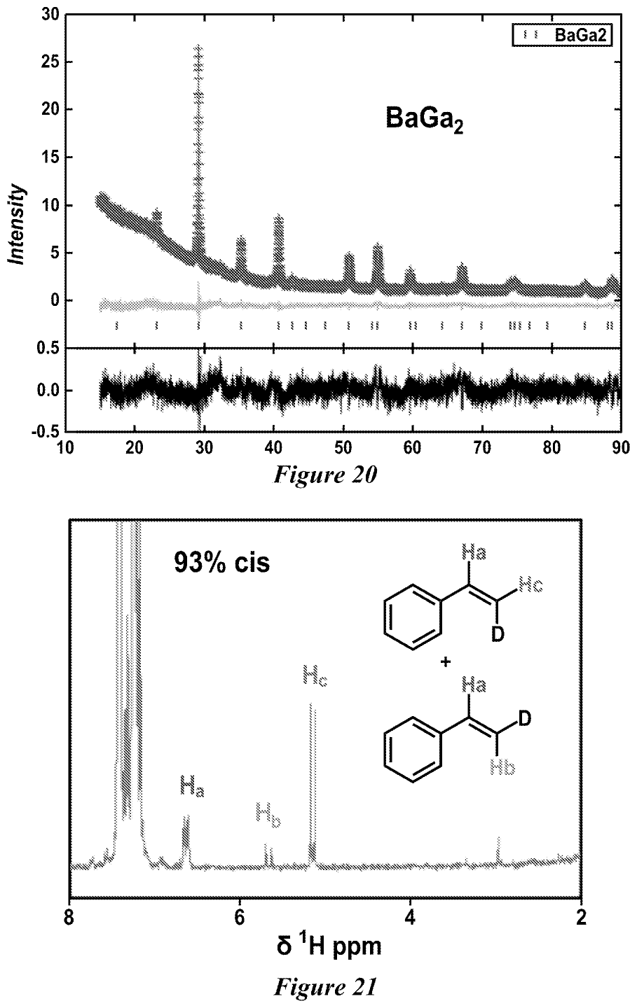

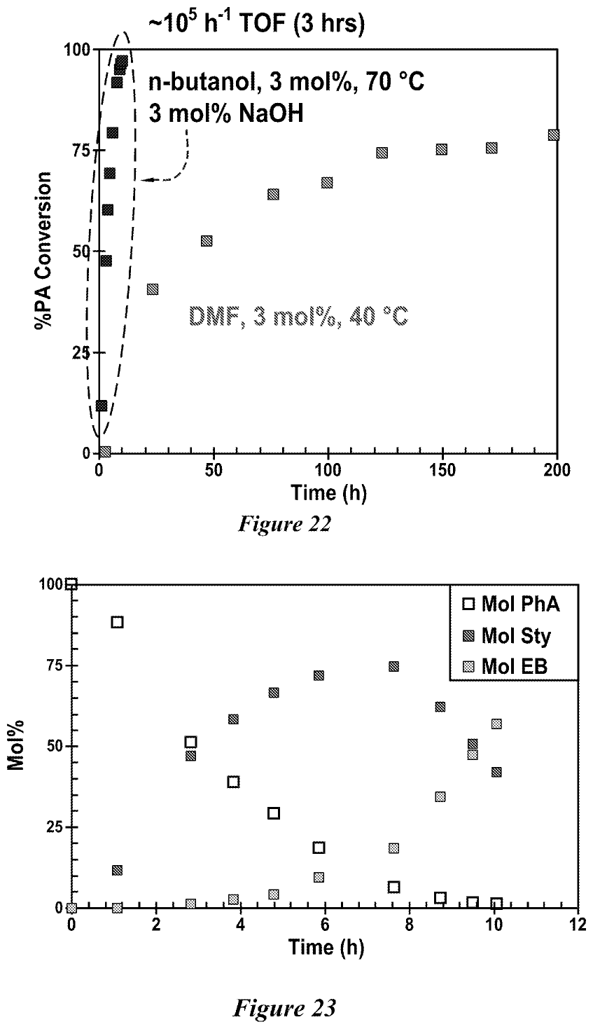

For example, BaGa₂ was used to catalyze the hydrogenation of phenylacetylene to styrene with a TOF of ˜392 h⁻¹ over 24 hours (FIG. 17-FIG. 21). The catalytic activity of BaGa₂ was further investigated using n-butanol/NaOH, Compared to the conversion of phenylacetylene in DMF at 40° C., the use of n-butanol/NaOH and a higher temperature gave accelerated conversion (˜10⁵ h⁻¹ TOF, 3 hrs) and even over hydrogenation (FIG. 22-FIG. 23).

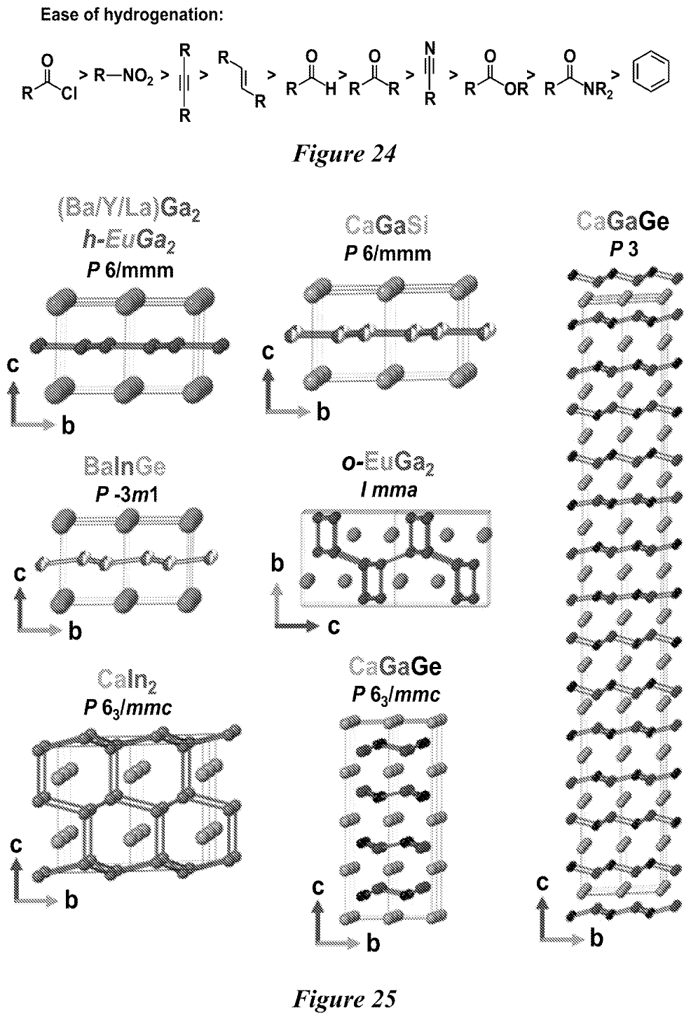

The catalysts described herein can be used for a variety of hydrogenation reactions (FIG. 24).

### Example 4

### Alkyne Hydrogenation Catalysis Across a Family of Ga/In Layered Zintl Phases

Transition metal-free Zintl-Klemm phases have received little attention as heterogeneous catalysis. Herein, it is shown that a large family of structurally and electronically similar layered Zintl-Klemm phases built from honeycomb layers of group 13 triel (Tr) or group 14 tetrel (rt) networks separated by electropositive cations (A) and having a stoichiometry of ATr₂ or ATrTt (A=Ca, Ba, Y, La, Eu; Tr=Ga, In; Tt=Si, Ge) also show varying degrees of activity for the hydrogenation of phenylacetylene to styrene and ethylbenzene at 51 bar H₂, 40-100° C. across a variety of solvents. The most active catalysts contain Ga with, formally, a half-filled p_{z} orbital, and minimal bonding between neighboring Tr₂ or TrTt layers. A 13-layer trigonal polytype of CaGaGe (13T-CaGaGe) was the most active, cyclable, and robust catalyst and under modest conditions (1 atm H₂, 40° C.) had a surface specific activity (590 h⁻¹) comparable to a commercial Lindlar's catalyst. Additionally, 13T-CaGaGe maintained 100% conversion of phenylacetylene to styrene at 51 bar H₂, even after 5 months of air exposure. This work reveals the structural design elements that can lead to particularly high catalytic activity in Zintl-Klemm phases, further establishing them as a promising materials platform for hydrogen-based heterogeneous catalysis.

Introduction. The design of new, inexpensive heterogeneous catalysts using earth abundant elements that can perform complementary chemical transformations to existing systems is essential for long term sustainability (Sheldon R A, J. R. Soc., Interface 2016, 13, 20160087; Ludwig J R et al. Chem 2017, 2, 313-316; Descorme C et al. ChemCatChem 2012, 4, 1897-1906). The need to limit the use of precious metals has spurned the discovery of new catalyst concepts and heterogeneous catalyst materials to maximize activity, stability, and selectivity, for which intermetallics have attracted considerable attention (Furukawa S et al. ACS Catal. 2017, 7, 735-765; Dasgupta A et al. Catal. Today 2019, 330, 2-15; Armbrüster M. Sci. Technol. Adv. Mater. 2020, 21, 303-322; Pei Y et al. J. Catal. 2019, 374, 136-142). For example, structurally precise intermetallic alloys such as GaPd₂ where precious metals are spatially separated to enhance selectivity, in accordance with the “active-site” isolation concept (Verbeek H S et al. J. Catal. 1976, 42, 257-267), were found to catalyze the semihydrogenation of acetylene to ethylene with high activities and selectivities (Armbruster M. Sci. Technol. Adv. Mater. 2020, 21, 303-322; Matselko O et at J. Phys. Chem. C 2018, 122, 21891-21896; Armbrüster M et al. J. Am. Chem. Soc. 2010, 132, 14745-14747; Grin Y et al. Mol. Phys. 2016, 114, 1250-1259; Kovnir K et al. Stud. Surf. Sci. Catal. 2006, 162, 481-488). As a second, example, many electrides, or compounds that contain excess electrons that preferentially occupy distinct crystallographic sites, have low work function that can readily reduce substrates such as H₂, and compounds such as LaCoSi have shown promise in NH₃ synthesis (Gong Y T et al. Nat. Catal. 2018, 1, 178-185).

Most of these intermetallic phases feature transition metals, due to the ease at which they can cycle between coordination environments and oxidation states facilitating the coordinative addition of substrates and elimination of products. Still, there are a large number of intermetallic phases that combine main group metals/metalloids with electropositive group 1-3 or lanthanide elements, that form structurally defined covalent networks in accordance with the Zintl-Klemm concept. These Zintl-Klemm phases have received virtually no attention as catalysts. It was recently discovered that BaGa₂ showed incredibly high catalytic activities for the semi hydrogenation of phenylacetylene to styrene and complete hydrogenation to ethylbenzene, despite the absence of transition metals (Hodge K L et al. J. Am. Chem. Soc. 2019, 141, 19969-19972). It was hypothesized that the origin of the catalytic activity stems from the electronic structure of BaGa₂ which causes a low barrier for the adsorption of alkynes and dissociative adsorption of H₂ (Hodge K L et al, J. Am. Chem. Soc. 2019, 141, 19969-19972), BaGa₂ crystallizes into the AlB₂ structure type (Björling T et al. J. Am. Chem. Soc. 2006, 128, 817-824). It comprises single atom thick honeycomb layers of Ga, with each layer separated by Ba atoms. This compound has a total valence electron count of 8 electrons per BaGa₂ formula unit, excluding the filled d¹⁰ shell. Every Ga has 3 covalent bonds with its neighbors and can be thought of as being formally reduced to Ga⁻ by Ba²⁺. Thus, the Ga⁻ network is isoelectronic to graphite; each Ga⁻ ion is sp²-hybridized, and forms a single π-bond with its neighbors. As π-bonding in 3^{rd} row and greater elements tends to be considerably weaker than σ-bonding, at 51 bar H₂ and above 170° C., BaGa₂ readily absorbs H₂ to form BaGa₂H₂ in which every Ga atom is terminated with a Ga—H bond (Björling T et al. J. Am. Chem. Soc, 2006, 128, 817-824), BaGa₂ was found to be a highly active alkyne hydrogenation catalyst, whereas BaGa₂H₂ was not (Hodge K L, et al. J. Am. Chem. Soc. 2019, 141, 19969-19972). This suggests that the presence of half-filled p₂ orbitals in layered honeycomb networks is an important motif for catalytic activity and for adsorption of the alkyne substrate on the surface. Remarkably, BaGa₂ showed surface specific activities that were within an order of magnitude of commercial Pd-catalysts and other transition-metal compounds (Hodge K L, et al. J. Am. Chem. Soc. 2019, 141, 19969-19972), Analysis of the supernatant immediately after the reaction showed <1 ppm Ga, highlighting the heterogeneous nature of the BaGa₂ catalyst (Hodge K L et al. J. Am. Chem. Soc. 2019, 141, 19969-19972). Unfortunately, upon exposure to H₂O or air, BaGa₂ instantaneously formed a surface oxide that inhibited its activity, thereby limiting its long-term use as a catalyst (Hodge K L et al, J. Am. Chem. Soc. 2019, .141, 19969-19972).

Once a new catalyst system is discovered, the systematic exploration of the catalytic behavior of numerous closely related analogues is essential for determining the balance between structural and electronic influences on the mechanism, selectivity, and activity to optimize catalytic performance (Meyer R J et al, ACS Catal. 2018, 8, 566-570). BaGa₂ is just one member in a larger family of Zintl-Klemm phase compounds containing honeycomb networks of main group elements separated by electropositive cations (Björling T et al, J. Am. Chem. Soc. 2006, 128, 817-824; Zapp N et al. Inorg. Chem. 2019, 58, 14635-14641; Wenderoth P et al. Inorg. Chem, 2013, 52, 10525-10531; Nuspl G et al, Inorg. Chem. 1996. 35, 6922-6932; Evans M J et al. Physical Review B 2009, 80, 064514; Bojin M D et al. Helv. Chim. Acta 2003, 86, 1683-1708; Bojin M D et al. Helv. Chim. Acta 2003, 86, 1653-1682). Based on the activity of BaGa₂ it seemed relevant to explore whether other members of this family in which at least one of the main group elements formally has a half-filled p_{z} orbital (7 valence electrons ignoring any π-bonding) could also display similar catalytic activity and would either be more resistant to oxidation or have a more readily removable oxide. It was hypothesized that this electronic motif would lead to surfaces with greater acidity, thereby enhancing the adsorption and subsequent hydrogenation of alkynes. In addition, whether the ability of a phase to absorb H₂ into its lattice and form a topotactic hydride, as well as whether the structural and electronic features of the compound would influence catalytic activity and oxide formation were probed. Such features include the planarity of network, the interlayer stacking sequence, whether one or both elements on the honeycomb network formally have 7 valence electrons, and if the electropositive cation is divalent or trivalent.

Herein, the catalytic activity of a series of Zintl phases built from honeycomb layers of group 13 triel (Tr) or group 14 tetrel (Tt) networks and having a stoichiometry of ATr₂, and ATrTt (A=Ca, Ba, Y, La, Eu; Tr=Ga, In; Tt=Si, Ge) were compared. These compounds have either 8 or 9 total valence electrons per ATr₂ or ATrTt formula unit. At least one of the triel elements in the network features a half-filled p_{z} orbital, which again, is considered to be an important electronic feature for alkyne hydrogenation. Most compounds have appreciable catalytic activity in the semihydrogenation of phenylacetylene to styrene at high pressure (51 bar H₂). The most active catalysts contained Ga and lacked any interlayer bonding in the main group framework, and there was no correlation between the ability to form a known hydride and catalytic activity. For many of these phases the activity gets disrupted upon exposure to air, due to surface oxidation. However, 13T-CaGaGe, YGa₂, and CaGaSi maintained activity after exposure to air. 13T-CaGaGe was the most active, cyclable and robust catalyst, and maintained 100% conversion of phenylacetylene to styrene at 51 bar H₂ even after 5 months of air exposure. Without air exposure this phase had surface specific activities (SSA) of 590 h⁻¹ at 1 atm H₂, and was selective for the hydrogenation of phenylacetylene to styrene, producing no detectable ethylbenzene within 24 hours. Taken together, this work shows that this broad family of layered Zintl phases having triel elements with half-filled p_{z} orbitals are catalytically active, outlining a new unexplored design feature for transition metal-free heterogeneous catalysts.

### Experimental Section

Preparation of Zintl Phase Catalysts. The synthesis of all compounds was performed using air-free conditions whenever possible, and all compounds and elements were prepared and stored in an Ar-filled glovebox.

For the synthesis of LaGa₂, oil from the lanthanum metal powder (99.9%, Strem) was removed by washing with hexanes several times and allowing it to dry. A total of 1.2 equivalents of lanthanum to 2 equivalents of gallium (99.99% Strem) were placed in an alumina crucible and sealed in an evacuated quartz tube. The tube was heated to 1100° C. over 10 hours and held at that temp for 18 hours with the furnace turned off at the end of the synthesis.

For the synthesis of orthorhombic EuGa₂ (o-EuGa₂), the oil and oxide on the outside of the europium surface were removed mechanically and 1.1 equivalents of europium (99.9%, Strem) to 2 equivalents of gallium were placed in crimped titanium foil and sealed in a quartz tube. The tube was heated in a vertical furnace over 8 hours to 900° C., held for 20 hours and then cooled over 12 hours.

For the synthesis of hexagonal EuGa₂ (h-EuGa₂), stoichiometric amounts of europium and gallium were heated in an alumina crucible in a sealed quartz tube to 1050° C. over 6 hours, held for about 16 hours and was rapidly quenched.

For the synthesis of BaInGe, stoichiometric amounts of indium, germanium, and a 5% excess of barium (99.7% Strem) were added to an alumina crucible and sealed in a quartz tube. The tube was heated up to 900° C. over six hours, ramped down to 850° C. over 18 hours with the furnace shut off after that time. To synthesize 13T-CaGaGe, CaIn₂, and CaGaSi, the surface oxide of the calcium turnings was mechanically removed. Stoichiometric amounts of Ca (99%, Sigma-Aldrich), Ga, Si (99.9999% Strem), In (99.9% Strem), and Ge were placed in a copper hearth and were arc-melted together. The resultant button was flipped so that the materials were melted a total of 5 times each. A similar process was followed for YGa₂ where stoichiometric amounts of yttrium (99.9% Strem) and gallium were arc-melted flipping between each melt so that button was melted a total of 5 times.

To synthesize 4H-CaGaGe, previously synthesized 1.3T-CaGaGe powder was sealed in an evacuated quartz tube and annealed at 500° C. for 18 hours.

### General Procedure for Catalytic Hydrogenations

In 1 bar pressure reactions, the catalytic material is ground in a mortar and pestle and weighed out in an Ar-filled glove box, then added to a 10 mL round bottom flask. Anhydrous solvents were then added to the flask. In a typical reaction ˜15 mg of catalyst was added to the flask with 2.5 mL of anhydrous solvent, followed by 0.9 mmol of phenylacetylene substrate. The sealed round bottom flasks were then removed from the glove box and placed on hot plates where a H₂ filled balloon was added. The H₂ pressure was assumed to be 1 bar. Elevated pressure reactions were done in a similar manner. In a typical reaction 1.8 mmol of substrate was added to a 20 mL vial along with 20-35 mg of catalyst and 2.5 mL solvent. These vials were placed in Parr reactors and once out of the glove box, hydrogen was added to the desired pressure (51 bar) and the reactors were placed on the heating elements.

Surface specific activities (SSAs) for balloon pressure reactions were calculated using equations 1, 2, and 3:

$$SSA = {n}_{0} C / {tn}_{cat} \qquad ( 1 )$$

$${n}_{cat} = {m}_{cat} {N}_{Ga sites} / {N}_{A} \qquad ( 2 )$$

$${N}_{Ga sites} = {S}_{BET} \times \frac{1 Ga atom}{{S}_{A}} \qquad ( 3 )$$

where C is the conversion of the substrate at time t, n₀ is the initial moles of substrate, neat is the maximum moles of Ga atoms exposed on the surface, meat is the mass of the catalyst, N_{Ga sites} is the amount of exposed gallium sites per gram of catalyst, N_{A} is Avogadro's constant, S_{BET} is the specific surface area of the material, and S_{A} is the surface area of the gallium atoms. CaGaGe has 1 Ga atom per 15.3 Å² of surface area for the 001 face of the unit cell, thus S_{A} would correspond to 15.3 Å² in equation 3.

When comparing catalytic activities between different solvents, all catalysts were used from the same synthetic batches to eliminate differences in the relative amount of oxides, impurities, and surface areas per gram, from synthesis to synthesis.

Characterization. Powder X-ray diffraction patterns were obtained at room temperature using a Balker D8 X-ray powder diffractometer (sealed Cu X-ray tube 40 kV and 40 mA) in Johansson mode.

High Performance Liquid Chromatography—A Shimadzu liquid chromatograph equipped used a reversed phase C18 column with solvents water and acetonitrile was used. Ratiometric calibration curves were made to identify ratios of phenylacetylene to styrene to ethylbenzene,

X-ray Photoelectron Spectra of the samples were taken with a Kratos Ultra X-ray photoelectron spectrometer with a monochromated Al X-ray Source. Non-air-exposed powders were prepared in an Ar-filled glovebox, affixed to a sample holder using carbon tape, and loaded into an air-free transfer tool, Energy calibration was performed using the C is peak as 284.8 eV,

Gas Adsorption Measurements: A Micrometrics 3Flex Surface Characterization Analyzer was used to measure single component, gas adsorption isotherms. The measurements were performed using ultra high purity krypton (99.999%) purchased from Praixair (Kr: 5.016-Q2000).

Prior to analysis 500 mg of 13T-CaGaGe was transferred to oven-dried sample tubes equipped with TransSeals™ (Micrometrics) and heated to 100° C. (1° C. min⁻¹) under vacuum until the outgas rate was less than 3 mbar min⁻¹. Surface areas were calculated from Kr adsorption isotherms (77K) by fitting the isotherm data to the BET equation with the pressure range (0.0001≤P/P₀≤0.4).

Results and Discussion. A series of Zintl phase catalysts with slightly varied structures and components were synthesized for the purpose of evaluating their comparative catalytic activities. The crystal structures of these catalysts are available in FIG. 25. LaGa₂, YGa₂, BaInGe, o-EuGa₂, h-EuGa₂, CaIn₂, CaGaSi, 4H-CaGaGe, and 13T-CaGaGe were synthesized to explore the effects of local structure, the presence of interlayer bonding, the potential to form topotactic hydride phases, valence electron count, and electronic structure on catalytic behavior and the propensity for oxidation compared to BaGa₂. Of all these compounds, only LaGa₂, BaGa₂; and BaInGe have been previously reported to react at 150-300° C., <80 bar H₂ or D₂ to form LaGa₂D_{0.7}, BaGa₂H₂, and BaInGeD, respectively (Björling T et al. J. Am. Chem. Soc. 2006, 128, 817-824; Werwein A et al. Crystals 2019, 9, 193; Evans M J et al. Inorg. Chem. 2009, 48, 5602-5604). In previous literature, either decomposition or no formation of hydride phases was observed under similar conditions with Caine (Björling T et al. J Am. Chem. Soc. 2006, 128, 817-824), o-EuGa₂ (Wenderoth P et al, Inorg. Chem. 2013, 52, 10525-10531; Werwein A et al. Crystals 2019, 9, 193), CaGaSi (Evans M J et al. J. Am. Chem. Soc. 2008, 130, 12139-12147), and 4H-CaGaGe (Evans M J et al. J. Am. Chem. Soc. 2008, 130, 12139-12147).

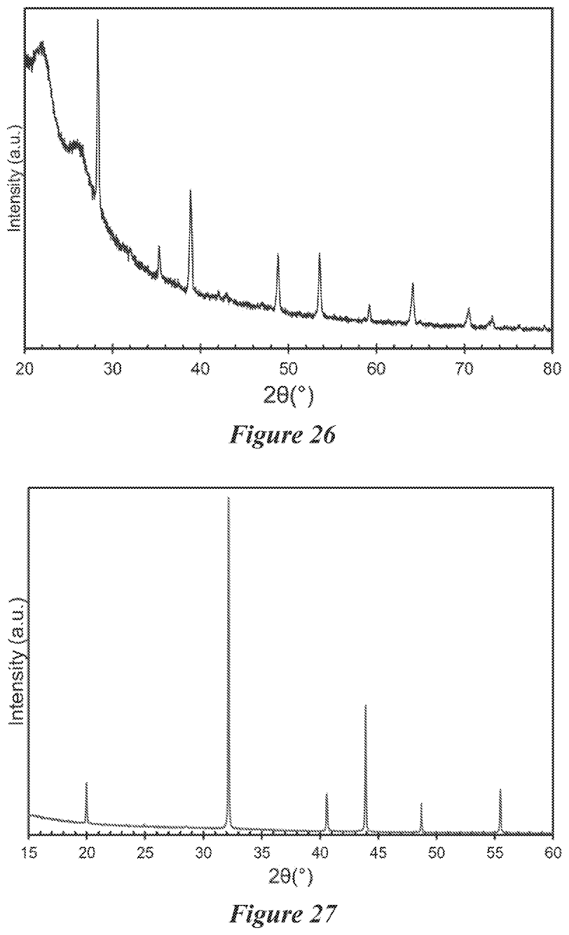

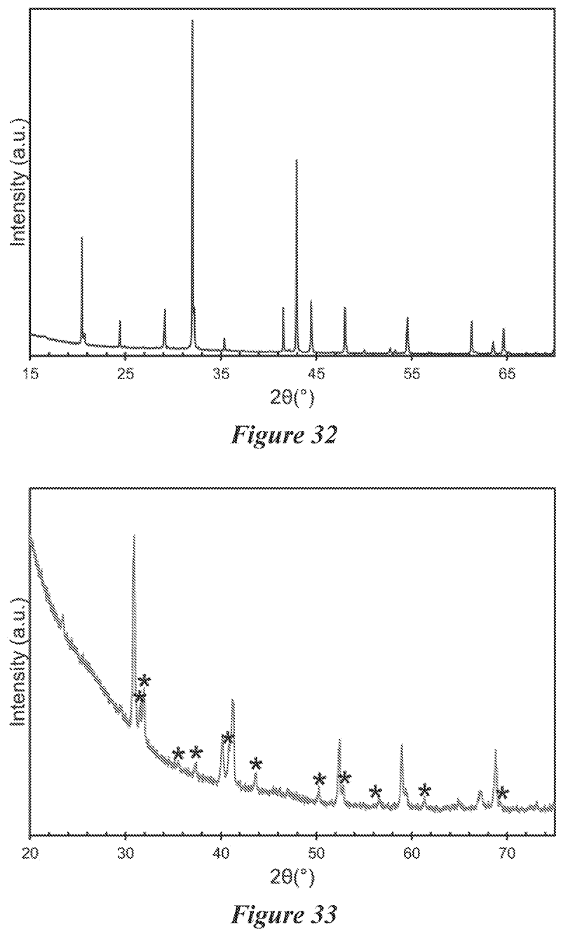

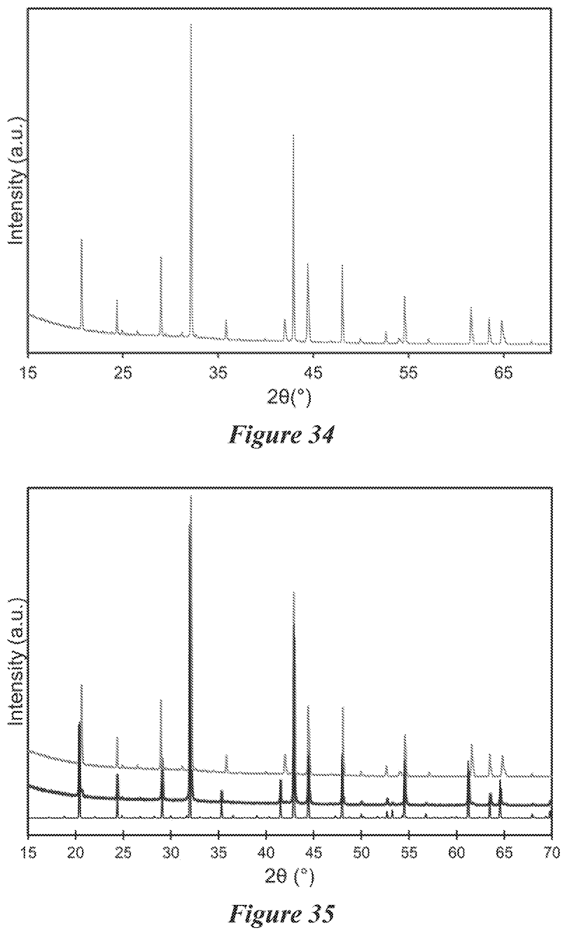

The powder diffraction patterns of all compounds are shown in FIG. 26-FIG. 35.

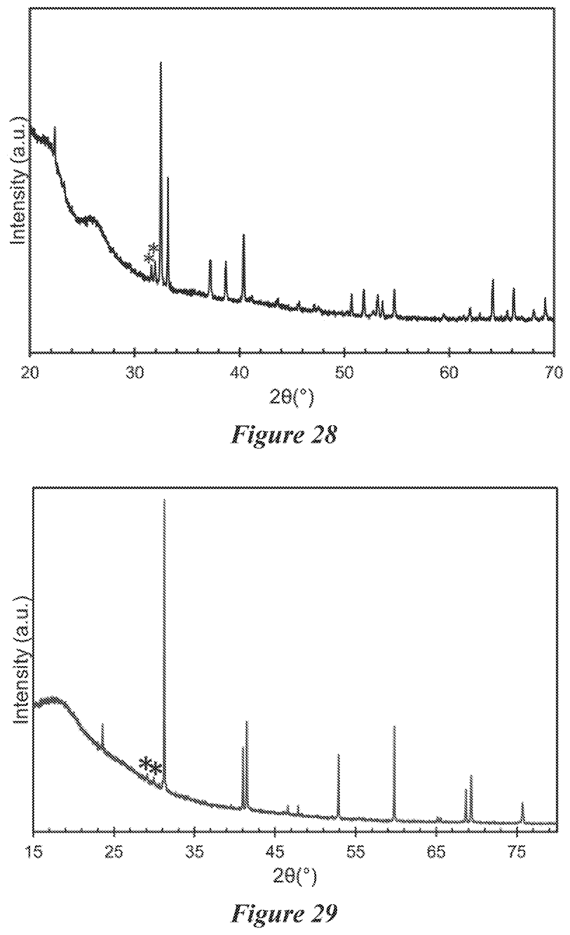

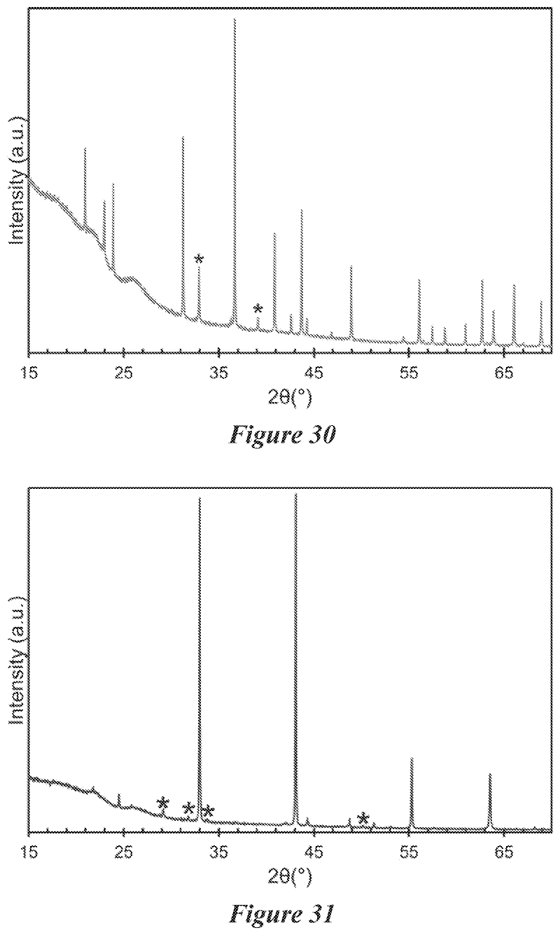

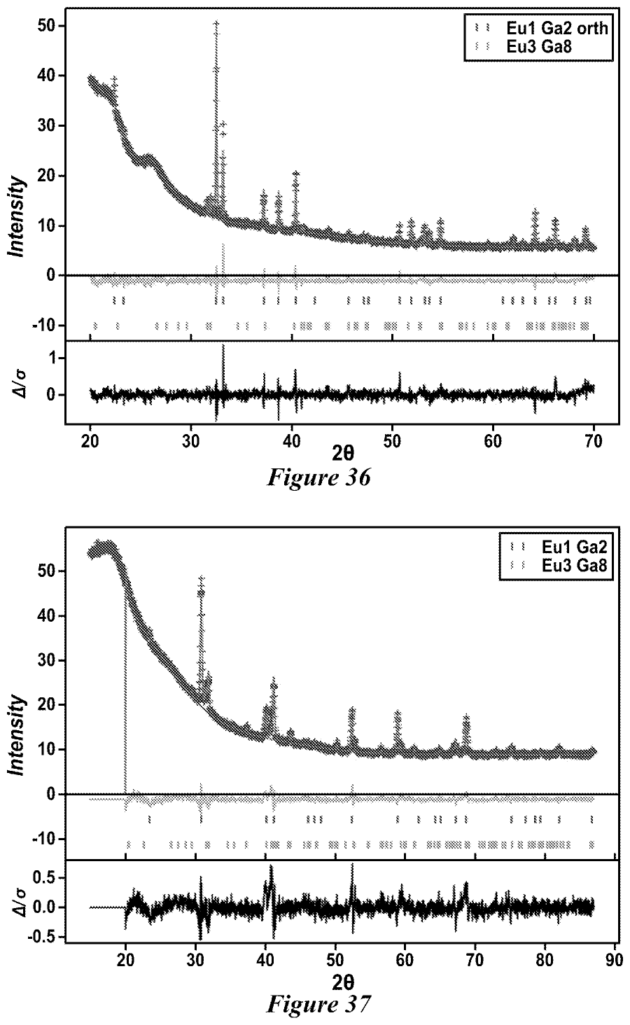

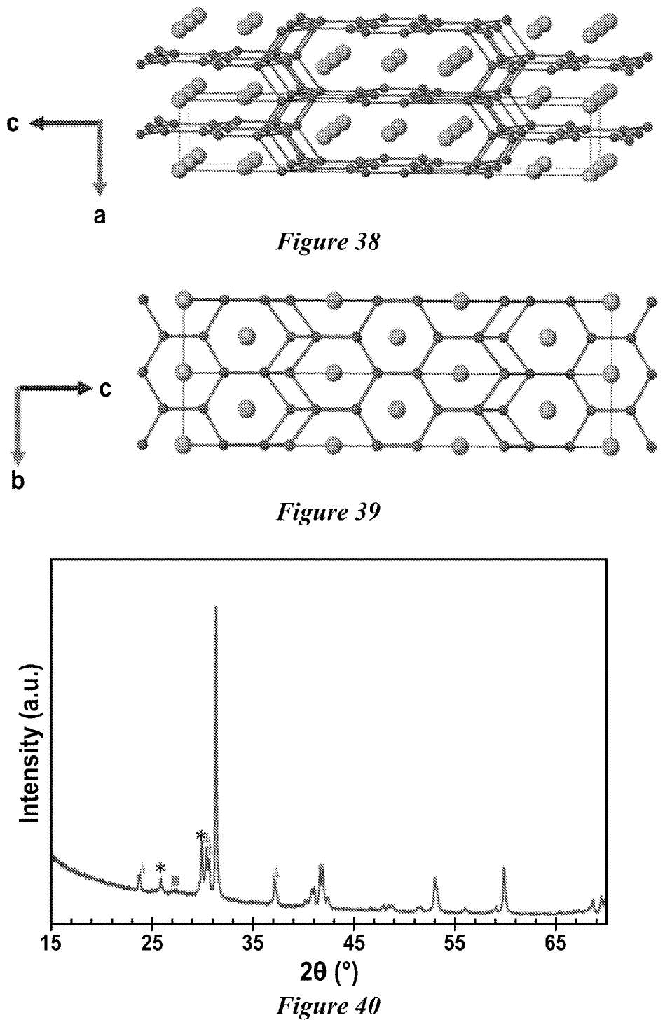

YGa₂, BalnGe, and CaGaSi, are all single-phase with no detectable impurity. LaGa₂ has a minor impurity of La₂O₃, which is attributed to its rapid propensity for oxidation (FIG. 29). YGa₂ has a trace impurity of Y₂O₃, which inherently exists on the surface of source Y precursor, and proved very challenging to remove (FIG. 31). CaIn₂ has a minor impurity of In (FIG. 30). BaGa₂ has a minor impurity of BaGa₄ which was previously showed to be catalytic inactive for alkyne hydrogenation (Hodge K L et al. J. Am. Chem. Soc. 2019, 141, 19969-19972). 13T-CaGaGe can only be accessed via arc-melting, and has ˜15% 4H-CaGaGe as a minor impurity phase. The discovery and elucidation of the structure of 13T-CaGaGe is reported below, Both o-EuGa₂ and h-EuGa₂ proved incredibly difficult to grow as pure phases. The h-EuGa₂ polymorph could only be formed via fast quenching from 1050° C., whereas the o-EuGa₂ polymorphs forms upon slow cooling in agreement with previous reports (Buschow K M et al. J. Less-Common Met. 1984, 97, L5-L8). However, both h-EuGa₂ and o-EuGa₂ always exhibited minor impurities of Eu₃Gas (10% and 8%, respectively) (FIG. 28, FIG. 33, FIG. 36, and FIG. 37). Interestingly, the crystal structure of Eu₃Ga₈ can be thought of as a layered intergrowth structure in which layers of EuGa₄ with tetrahedral Ga, are connected to planar honeycomb layers of Ga in h-EuGa₂ (FIG. 38-FIG. 39) (Demooij D B et al. J. Less-Common Met. 1985, 109, 117-122). It has been established that Eu is divalent in all these compounds (De Vries J W C et al. Physica B+C 1985, 128, 265-272). In other words, ⅔ of the Ga atoms in the Eu₃Ga₈ are sp²-hybridized with formally 7 valence electrons and have the same local planar honeycomb coordination environment as h-EuGa₂, which, again, is hypothesized to be a structural and electronic feature indicative of catalytic activity (Demooij D B et al. J. Less-Common Met. 1985, 109, 117-122).

By changing the electropositive element in BaGa₂ from barium to europium, it was possible to explore how a phase that is isoelectronic but having a different bonding behaves catalytically. In particular, the fact that h-EuGa₂ is isostructural to BaGa₂ whereas o-EuGa₂ is not, gives excellent insight into how the different bonding networks behave catalytically. The o-EuGa₂ phase features divalent Eu²⁺ and is distorted into an orthorhombic lattice, in which the intralayer Ga—Ga distance ranges from 2.65-2.70 Å while the interlayer Ga—Ga distances are 2.81 Å (Demooij D B et al, J. Less-Common Met. 1985, 109, 117-122), Compared to the intralayer and interlayer distances of BaGa₂ (2.55 Å and 5.072 Å, respectively) and h-EuGa₂ (2.51 Å and 4.51 Å, respectively), it is clear that o-EuGa₂ exhibits appreciable interlayer Ga—Ga bonding, CaIn₂ is another phase with significant interlayer bonding. This hexagonal phase has significant puckering in the honeycomb In framework. The intralayer In—In distances are 2.92 Å, while the interlayer distances are 3.13 Å, which is also in the range expected for a covalent In—In bond (Nuspl G et al. Inorg. Chem. 1996, 35, 6922-6932). Beyond the more subtle structural differences, the group 13 element is changed from gallium to indium to probe its influence on catalysis.

The remaining compounds synthesized all allowed for the exploration of the influence of having one additional valence electron on the honeycomb framework, which would result in only one of the two main group elements to have a half-filled p_{z} orbital, rather than both. LaGa₂ and YGa₂ are isostructural to BaGa₂ and have planar honeycomb Ga layers. Replacing divalent Ba²⁺ with trivalent Y³⁺ and La³⁺ gives one more electron per honeycomb layer according to Zintl-Klemm counting rules and 9 total valence electrons per formula unit. The ATrTt phases in which A is a divalent cation also feature 9 electrons per formula unit. CaGaSi has a planar honeycomb network, but with a random configuration of gallium and silicon atoms on the framework. In BaInGe, the In and Ge atoms also randomly occupy both sites of the honeycomb layers, which are slightly distorted away from planarity having In/Ge—In/Ge—In/Ge bond angles of 117.8°. Finally, in CaGaGe the Ga and Ge atoms occupy distinct positions on the framework and also distort away from planarity. CaGaGe forms two different polytypes, ‘4H-CaGaGe and 13T-CaGaGe that are closely related. 4H-CaGaGe features puckered honeycomb layers with the Ga and Ge atoms occupying distinct sites. The Ga atoms in neighboring layers are directly on top of each other and are tilted towards each other. Still, the interlayer Ga—Ga distance is 3.577 Å indicating minimal bonding interactions of the half-filled p_{z} orbitals. The 13T-CaGaGe structure can be thought of as 3 unit cells of 4H-CaGaGe, with a 13^{th} misaligned layer, and has interlayer Ga—Ga distances that range from 3.22 to 3.62 Å (with the exception of the misfit layer which is 4.43 Å). These are much larger than the 2.81 Å interlayer Ga—Ga distances in o-EuGa₂, for which interlayer bonding occurs. Thus, 13T-CaGaGe similarly has minimal interlayer bonding interactions between Ga atoms. In addition, the Ga—Ge—Ga bond angles within each honeycomb layer are slightly distorted away from planarity and range from 111.8-117.6° in the 13T phase, and are 115.2’ in the 4H phase.

The catalytic activity of the different synthesized phases were screened for the semihydrogenation and hydrogenation of phenylacetylene to styrene and ethylbenzene at 51 bar H₂, with 20-35 mg catalyst (3.6-8.5 mol %), and under different solvents (n-butanol (n-BuOH), dimethylformamide (DMF), N-methyl pyrrolidone (NMP)) without exposing these catalysts to air (Table 5). Most compounds showed some degree of alkyne semihydrogenation compared to control experiments with no catalyst, which showed less than 5% conversion. This allowed for general trends that correlate structure and activity to be elucidated. First, the Zintl phases with interlayer bonding between the main group elements had the least catalytic activity. CaIn₂ and o-EuGa₂ have interlayer and Ga—Ga distances within the range expected for covalent bonds, but showed the least activity across all three solvents. Considering h-EuGa₂ is much more catalytically active than o-EuGa₂ yet contains the same elements, the differences in catalytic activity can be explained by their differences in electronic structure. In particular, the presence of half-filled p_{z} orbitals in the honeycomb network, as occurs in h-EuGa₂, is an important electronic motif for activity. Furthermore, it is unclear if the trace activity in o-EuGa₂ is caused by the Eu₃Gag impurity, which is present in both compounds, as its crystal structure also contains the same planar honeycomb Ga networks with half-filled 3p orbitals as occurs in h-EuGa_{2.} The only Zintl-Klemm phase with less activity than CaIn₂ and o-EuGa₂ without interlayer bonding was LaGa₂. The lack of activity of LaGa₂ can be explained by its extreme air-sensitivity, as it is the only phase that rapidly oxidizes upon immediate exposure to air (FIG. 40).

TABLE 5
The percent conversion of phenylacetylene to styrene (ST) and ethylbenzene (EB) after 20 h in 51
bar H₂, 2.5 mL of n-BuOH, NMP, or DMF solvent, 55-90° C., 20-35 mg catalyst (3.6-8.5 mol %),
1.82 mmol phenylacetylene, for 20 h. Unless otherwise noted, the sole hydrogenation product was
ST. The same conditions were used to test for catalysis after the powder was exposed to air for 24 h.

| Catalyst | % Conversion | % Conversion | % Conversion | After 24 h |
|---|---|---|---|---|
| (typical mol %) | n-BuOH, 90° C. | NMP, 55° C. | DMF, 55° C. | air exposure |
| No Catalyst | <5% | <5% | <5% | — |
| LaGa₂ (4.7-5.5%) | <5% | <5% | <5% | <5% (DMF, 55° C.) |
| CaIn₂ (4.4-4.9%) | 11% | 43% | 31% | 6% (DMF, 55° C.) |
| o-EuGa₂ (3.8-3.9%) | 20% | <5% | <5% | 9% (n-BuOH, 90° C.) |
| h-EuGa₂ (6.9%) | 63% | — | — | — |
| BaInGe (4.9-5.5%) | 6% | 23% | 31% | <5% (DMF, 55° C.) |
| CaGaSi (8.1-8.5%) | 58% | 39.5% | 67% | 33% (DMF, 55° C.) |
| | 54.5% ST, 3.5% EB | 31% ST, 8.5% EB | 67% ST, 0% EB | 33% ST, 0% EB |
| YGa₂ (3.6-5.4%) | 55% | 96% | 62% | 21% (DMF, 55° C.) |
| | 55% ST, 0% EB | 96% ST, 0% EB | 62% ST, 0% EB | 21% ST, 0% EB |
| BaGa₂ (4.5-5.5%) | 95% (1) | <5% | 100% (1) | <5% (all) |
| | 58% ST, 37% EB | | 0% ST, 100% EB | — |
| 13T CaGaGe (6.2-8.1%) | 100% | 98.5% | 100% | 100% (NMP, 55° C.) |
| | | | | 100% (n-BuOH, 90° C.) |
| | 85% ST, 15% EB | 0.6% ST, 97.9% EB | 0% ST, 100% EB | n-BuOH: 98% ST, |
| | | | | 2% EB NMP: 0.5% ST, 99.5% EB |
| 4H CaGaGe | 55% (2) | — | 36% (2) | — |

The remaining compounds studied had appreciable activity in the high pressure hydrogenation in n-BuOH, DMF, and NMP. BaInGe showed some activity for the hydrogenation of phenylacetylene, but it was lower than all the gallium compounds. It might be the general case that In-containing phases have reduced activity compared to Ga-containing phases. However, BaInGe is the only known layered honeycomb phase containing indium with a similar electronic structure (i.e., formally having a half-filled In 4p_{z} orbital). Still, BaGa₂, CaGaSi, YGa₂, and 13T/4H-CaGaGe all outperform BaInGe, which helps support the idea that indium is less active.

The four most active catalysts were CaGaSi, YGa₂, BaGa₂, and 13T-CaGaGe. These compounds have different total valence electrons per formula unit and are either planar or distorted. BaGa₂ features 8 total valence electrons per formula unit, whereas the other phases have 9 total valence electrons per formula unit, thereby indicating that high catalytic activity can be achieved when either one or both of the main group elements on the framework feature a half-filled p_{z} orbital. Additionally, 13T-CaGaGe has a puckered honeycomb arrangement of Ga—Ge atoms, whereas CaGaSi, BaGa₂, and YGa₂ are all planar, suggesting that catalytic activity does not depend on whether there are small distortions away from planarity. This is consistent with previous reports that indicate that the electronic structure of the active site and not the geometric structure primarily influences catalytic activity (Armbrüster M. Sci. Technol. Adv. Mater. 2020, 21, 303-322). Finally, there is no direct correlation between the high catalytic activity observed in these four compounds and whether the Zintl-Klemm phase can react with H₂ at moderate temperatures and pressures to form a topotactic hydride, as CaGaSi is reported to be unable to form CaGaSiH, whereas BaGa₂ can form BaGa₂H₂ (Björling T et al. J. Am. Chem. Soc. 2006, 128, 817-824; Evans M J et al. J. Am. Chem. Soc. 2008, 130, 12139-12147).

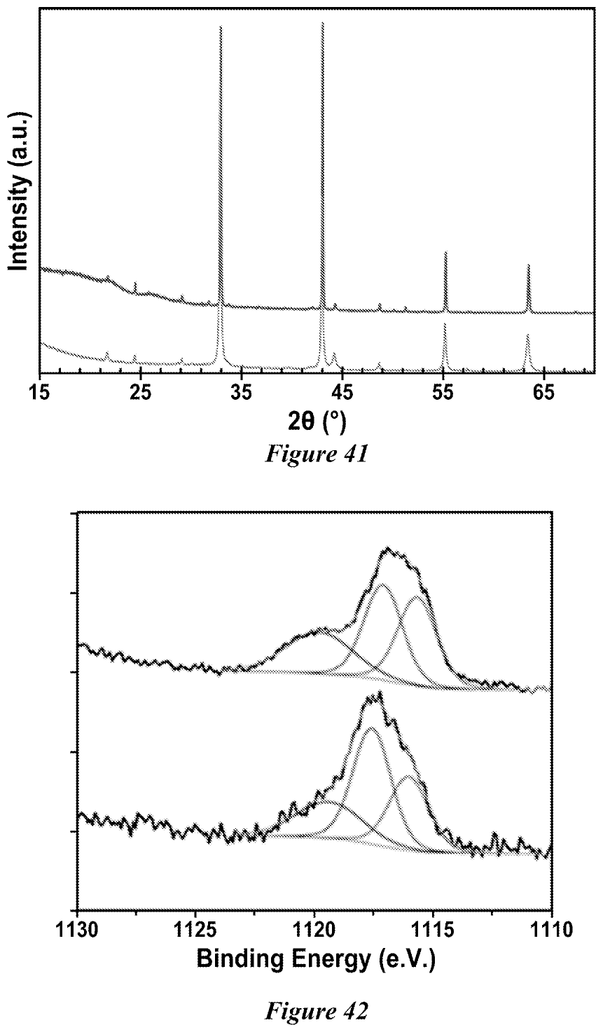

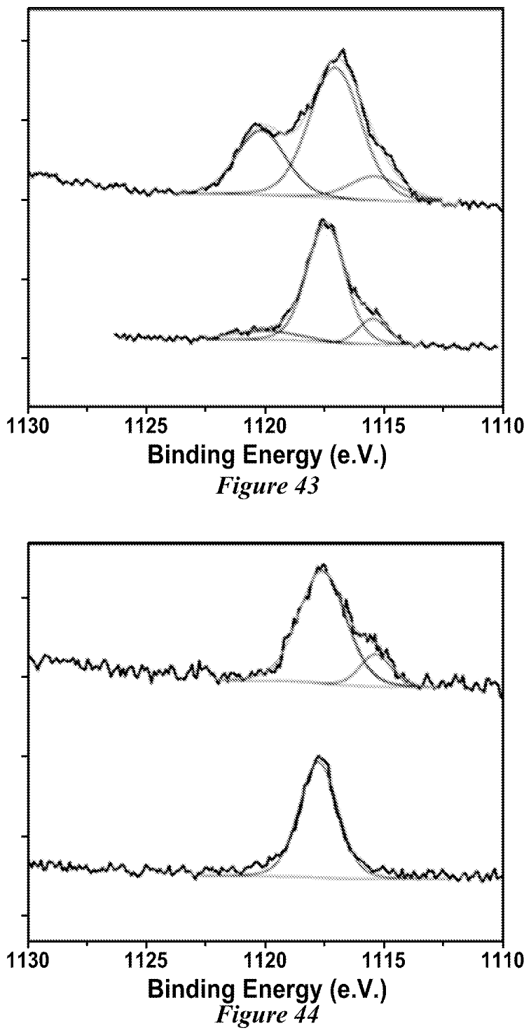

Next, how the catalytic properties of these compounds changed upon exposure to air was probed. While as a general rule, Zintl-Klemm phases tend to be air sensitive and prone to surface oxidation due to the reduced nature of the main group element, the thermodynamic propensity and rate of oxidation of the surface and in the bulk can significantly vary between compounds. The presence of oxide phases was not detected in the XRD patterns for almost all phases after exposure to air, with the exception of LaGa₂, which starts to form La(OH)₃ and Ga₂O₃ impurities after 2 hours of exposure to air (FIG. 40). The trace Y₂O₃ impurity in YGa₂ can be attributed to be a result of the surface oxide on the Y source, as the intensity of the Y₂O₃ reflections in the XRD pattern do not increase after air exposure (FIG. 41). Still, most compounds had a drop off in catalytic activity upon exposure to air for 1 day, implying the formation of a surface oxide layer disrupts catalysis. XPS was used to compare the changes to the surface of the most active gallium compounds, 13T-CaGaGe, Y-Ga₂, and BaGa₂ before and after their exposure to air (FIG. 42-FIG. 44), focusing on the Ga 2p_{3/2} peak. Before exposure to air, when all compounds are highly active, all three compounds have a 2p3/2 low energy shoulder centered at 1115.4-1115.7 eV that are indicative of an anionic Ga species, as would be expected in these layered Zintl-Klemm phases. All compounds also exhibit dominant 2p_{3/2} peaks centered at ˜1117.1-1117.9 eV, and 13T-CaGaGe and YGa₂ featured a more oxidized peak centered at 1119.8-1120.2. Both of these peaks correspond to the varying degrees of Gaⁿ⁺ surface oxidation that may occur with the native oxide (Romo-Garcia F et al. Opt. Mater. Express 2019, 9, 4187-4193; Lin Y J et al. Appl. Phys. Lett. 2000. 77, 687-689; Cybulskis V J et al. Catal. Lett. 2017, 147, 1252-1262). The presence of the low energy shoulder was previously found to be an important indicator for catalytic activity in BaGa₂ (Hodge K L et al. J. Am. Chem. Soc. 2019, 141, 19969-19972), The relative area of this low energy shoulder before air exposure is 16%, 34% and 11% for BaGa₂, 13T-CaGaGe, and YGa₂, respectively. Upon exposure to air for 2 h, the low energy shoulder disappears in BaGa₂, however, it still remains prevalent in both 13T-CaGaGe and YGa₂ at 24%, and 12%, respectively. YGa₂ and 13T-CaGaGe both show catalytic activity upon air-exposure, while BaGa₂ does not, further showing that the low energy shoulder correlates with catalytic activity in these other phases, and that the different Zintl phases can have differing rates of surface oxidation.

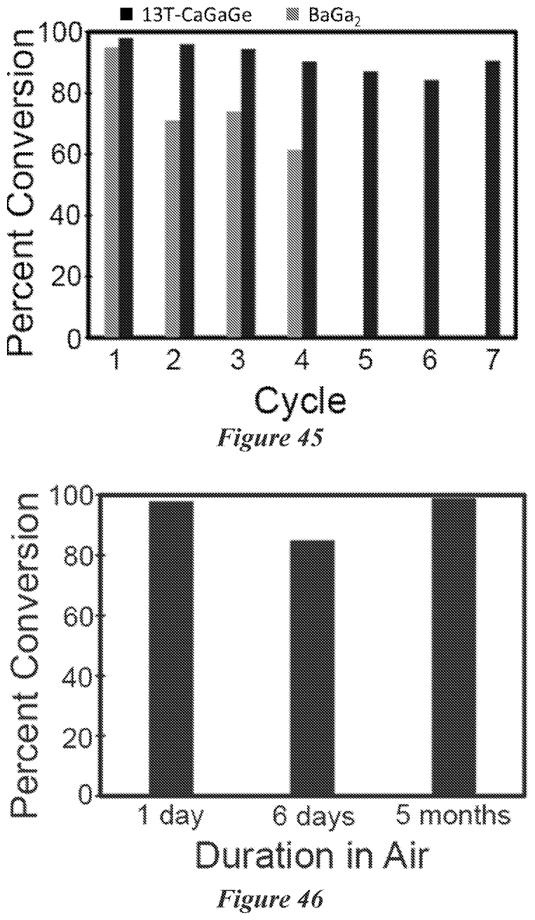

13T-CaGaGe is by far the most active Zintl-Klemm catalyst demonstrating complete semihydrogenation from phenylacetylene to styrene in n-BuOH and complete hydrogenation to ethylbenzene in NMP and DMF, in the initial 51 bar H₂ tests. What is most remarkable about this phase is its enhanced air-stability and long-term recyclability. The recyclability of the same 13T-Cat catalyst was probed in phenylacetylene semihydrogenation after seven 24 h cycles at 51 bar H₂ in n-BuOH (FIG. 45). When switching reagents, the same catalyst was exposed to air between each cycle for durations of 2-24 h. Compared to BaGa₂, which would lose all catalytic activity upon instantaneous air-exposure, 13T-CaGaGe maintains a much higher conversion with more cycles, and even has 90% conversion after seven cycles (FIG. 45). To further probe the air-stability of this catalyst, it was explored whether any changes in percent conversion occurred after exposing to air after 1 day, 6 days, and 5 months, using the same 51 bar reaction conditions (FIG. 46). Remarkably; —100% conversion to styrene is observed even after 5 months of exposure to air (FIG. 46). This resilience of the catalytic activity to prolonged air exposure is unprecedented among these Zintl-Klemm phases.

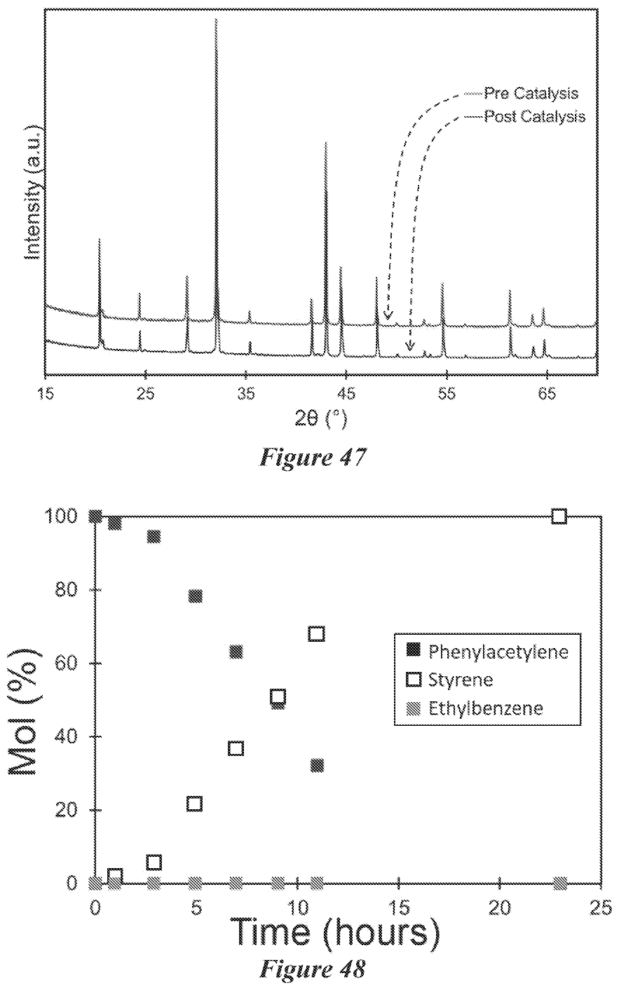

The XRD patterns of 13T-CaGaGe before and after catalysis are shown in FIG. 47.

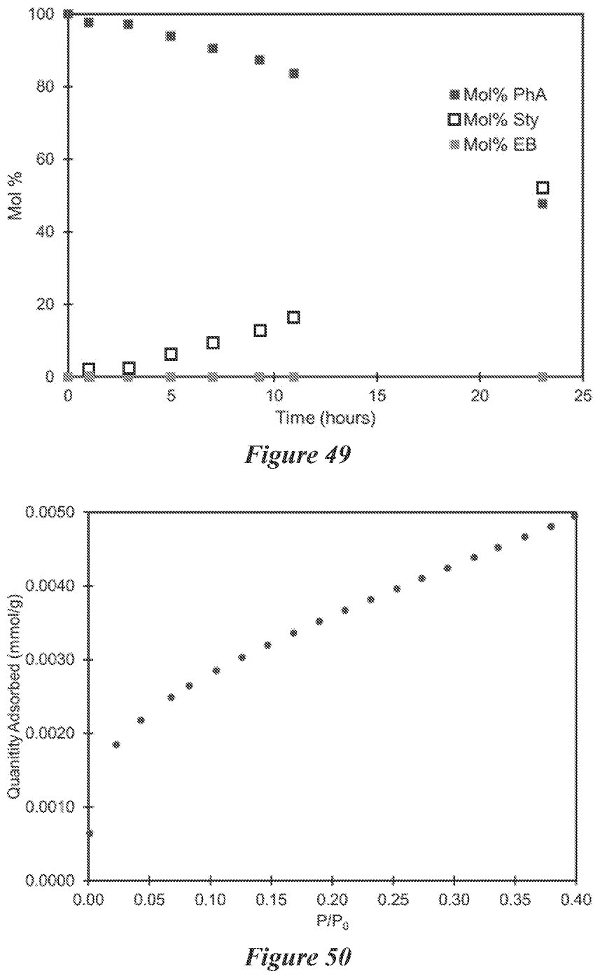

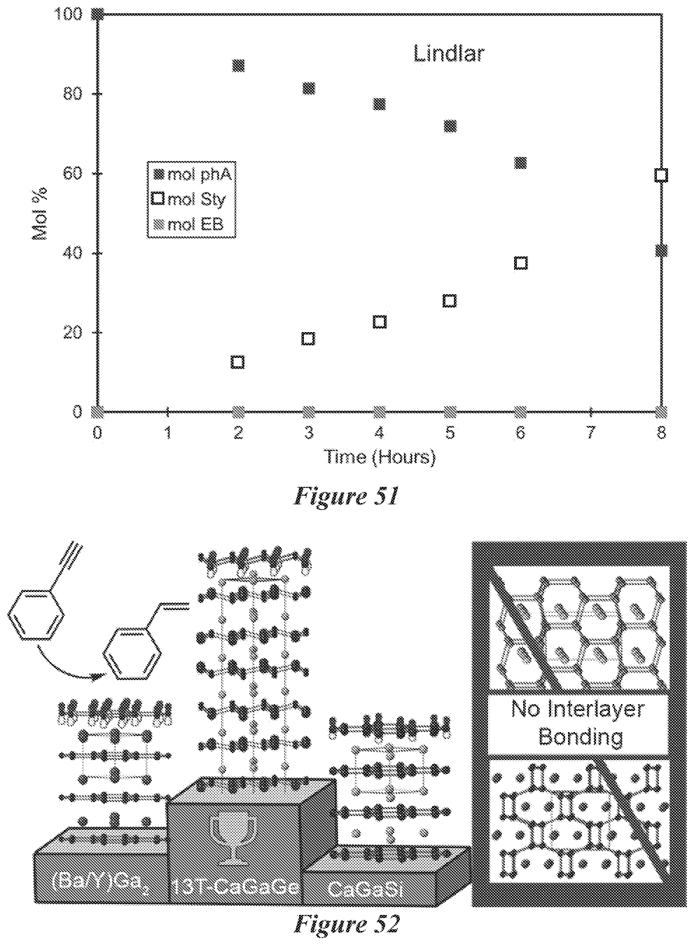

13T-CaGaGe also catalyzes the semihydrogenation of phenylacetylene to styrene under much milder conditions (1 bar H₂, 40° C., 8.5 mol % 13T-CaGaGe, 23 h), again with no detectable ethylbenzene formation. Catalytic activity was observed in a variety of solvents at 1 bar H₂ (Table 6), with complete conversion occurring in anhydrous NMP, with non-air exposed catalyst. The next best solvents are n-BuOH and DMF, for which >50% conversion of phenylacetylene to styrene was achieved after 23 h. As shown in the conversion vs. time (FIG. 48), complete conversion of phenylacetylene to styrene occurred well within 23 h in NMP. The time dependent conversion in DMF is shown in FIG. 49 and highlights a steady but reduced catalytic activity in this second solvent. The Surface Specific Activity (SSA) in NMP was determined by measuring the surface area per gram of catalyst using the Kr adsorption isotherm analyzed via the Braunauer-Emmett-Teller (BET) method (FIG. 50), along with the conversion vs time. The surface area of 13T-CaGaGe was determined to be 0.406 m² g⁻¹. Assuming that Ga is the only catalytically active site, the specific surface activity is estimated to be 590 h⁻¹, using the 5 h time point in FIG. 48. It is important to point out that in both solvents, the time dependent conversion indicates the presence of an induction period, which makes the estimate of specific surface activity a lower bound, and significantly complicates further kinetic analysis. An induction period was also similarly observed in BaGa₂, but ICP-OES analysis of the supernatant indicated the amount of soluble Ga to be <1 ppm, suggesting that catalysis is not due to a soluble Ga based impurity (Hodge K L et al. J. Am. Chem. Soc. 2019, 141, 19969-19972). The 13T-CaGaGe specific surface activity is higher than what was previously reported for BaGa₂ (425 h⁻¹), and is relatively close to a Lindlar's catalyst control (650 h⁻¹) (FIG. 51). Thus, 13T CaGaGe dethrones BaGa₂ as the most active Pt/Pd-free heterogeneous catalyst for phenylacetylene hydrogenation and has comparable activities with Pd. For comparison, the specific surface activities of other phenylacetylene hydrogenation catalysts under similar conditions are listed in Table 7.

TABLE 6
Conversion of phenylacetylene hydrogenation to styrene in
the listed anhydrous solvents (40° C., 23 h, 8.5 mol
% (~15 mg) 13T-CaGaGe catalyst, 0.91 mmol phenylacetylene)

| Solvent | Conversion (%) |
|---|---|
| NMP | 100 |
| n-butanol | 60 |
| DMF | 52 |
| 1,4-dioxane | 14 |
| IPA | 18 |
| ethanol | 23 |

TABLE 7
Comparison in activity for different catalysts for
the conversion of phenylacetylene to styrene.

| | | Surface activity or | |
|---|---|---|---|
| | | activity per mole of | |
| Catalyst | Conditions | catalyst | Reference |
| CaGaGe | 1 Bar H₂. | 590 h⁻¹ | This Work |
| | 40° C., NMP | (per surface Ga) | |
| CaGaGe | 1 Bar H₂. | 170 h⁻¹ | This work |
| | 40° C., DMF | (per surface Ga) | |
| BaGa₂ | 51 Bar H₂. | 8390 h⁻¹ | Hodge K L et al. J. Am. Chem. |
| | 40° C., DMF | (per surface Ga) | Soc. 2019, 147, 19969-19972 |
| BaGa₂ | 1 Bar H₂. | 425 h⁻¹ | Hodge K L et al. J. Am. Chem. |
| | 40° C., DMF | (per surface Ga) | Soc. 2019, 141, 19969-19972 |
| Lindlar's | 1 Bar H₂. | 650 h⁻¹ | This work |
| Catalyst | 40° C., DMF | (per mol Pd) | |
| Ni₃Ga | 5 bar H₂ | 5.16 × 10⁻³ h⁻¹ | Liu Y et al. Adv. Mater. |
| | 40° C., hexanes | (per surface Ni) | 2016, 28, 4747-4754 |
| Au/Graphene | H₂ flow (0.2 mL/min), | 360 h⁻¹ | Shao L et al. ACS Catal. |
| Oxide | 60° C., Ethanol | (per surface Au) | 2014, 4, 2369-2373 |
| PdZn/ZnO | 6 bar H₂, | 15840 h⁻¹ | Yoshida H et al. RSC Adv. |
| | 0° C., toluene | (per surface Pd) | 2014, 4, 24922 |
| Pd/Carbon | H₂ flow (30 mL/min) | 3600 h⁻¹ | Dominguez-Dominguez S et al. |
| Nanotubes | 50° C., Methanol | (per surface Pd) | J. Phys. Chem. C 2008, |
| | | | 112, 3827-3834 |
| Lindlar's | Continuous flow, 1 bar | 1866 h⁻¹ | Kuwahara Y et al. ACS Catal. |
| Catalyst | H₂, methanol, | (per surface Pd) | 2019, 9, 1993-2006 |
| | 1,4-dioxane, 30° C. | | |
| Pd/FDU-12 | 1 bar H₂, | 1840 h⁻¹ | Guo M et al. ACS Catal. |
| | 25° C., Ethanol | (per surface Pd) | 2018, 8, 6476-6485 |

Conclusion. This work shows that a broad of family of electronic and structurally similar layered Zintl-Klemm ATr₂ and ATrTt phases can catalyze the hydrogenation and semihydrogenation of phenylacetylene with varying activities. The common feature in the electronic structure of these materials that is important for catalytic activity is the presence of a triel element on the honeycomb lattice that would formally have a half-filled p_{z} orbital, bestowing acidity onto the framework. The most active catalysts lacked any interlayer bonding (FIG. 52). Furthermore, the ability to form bulk metal hydride phases did not correlate with activity. While all compounds lost some degree of catalytic activity upon exposure to air, 13T-CaGaGe remained highly active in high-pressure hydrogenation reactions. Without exposure to air, 13T-CaGaGe had specific surface activities comparable to Lindlar's catalyst at 1 bar H₂, and were among the largest for non-Pd/Pt catalysts. This discovery that this family of materials universally catalyze alkyne hydrogenation, paves the way for future surface science studies on the origin of catalytic behavior in these compounds, as well as expansion of the reaction and substrate scope, and optimization of long-term catalytic performance.

### Example 5

### Lucky Number 13: A 13-Layer Polytype of the Alkyne Hydrogenation Catalyst, CaGaGe

Abstract: The ability of materials to form crystal structures with different stacking sequences, known as polytypism, occasionally causes materials with the same stoichiometry and similar local structures to have profoundly different properties. Herein, a metastable 13-layer trigonal (13T) polytype of CaGaGe, a layered intermetallic phase comprised of [GaGe]²⁻ honeycomb networks separated by Ca²⁺, is discussed. This 13T polytype is readily synthesized from arc-melting the elements, and its structure is elucidated via neutron diffraction. This air-stable polytype has one-misaligned [GaGe]²⁻ layer for every 13, and transforms into the more stable 4-layer hexagonal (4H) CaGaGe polytype after annealing at 500° C. This transition metal-free 13T CaGaGe shows remarkable activity in the catalytic hydrogenation of phenylacetylene to styrene and ethylbenzene, whereas the activity of the 4H polytype is reduced. This work identifies the first 13-layer polytype for any crystal structure, and further establishes the influence of polytypism on catalysis.

Introduction: Polymorphism, the ability for compounds with the same stoichiometry to have different crystal structures, is quite prevalent in materials chemistry. Very often polymorphs have significantly different local bonding networks (i.e., graphite vs. diamond), which leads to dramatically different properties just as with constitutional isomerism. Polytypism is a subset of polymorphism commonly occurring in layered compounds that describes materials having a nearly identical structure and composition along two dimensions but are stacked into unit cells with different repeating sequences. While most polytypes generally lead to subtle differences in electronic structure and properties, sometimes dramatic changes are observed. For instance, the layered van der Waals compound, TaSe₂₋ₓTeₓ, exhibits an order of magnitude higher superconducting transition temperature when it stacks into a 3-layer rhombohedral unit cell polytype compared to a 2-layer hexagonal polytype, despite minimal differences in the local coordination (Luo H X et al. Proc. Natl. Acad. Sc. U.S.A. 2015, 112, 1-7). SiC can form many different polytypes of layers of close-packed corner sharing tetrahedra, and its band gap changes from 2.39 to 3.33 eV in the zinc-blende and wurtzite stacking sequences, respectively (Park C H et al. Phys. Rev. B: Condens. Matter Mater. Phys. 1994, 49, 4485-4493). As a final example, there have been significant differences in the catalytic and electrocatalytic behavior between the face centered cubic (3C) and the 4-layer hexagonal (4H) polytypes of gold and copper, which have different arrangements of close packed layers (Chen Y et al, Adv. Mater. 2017, 29, 1701331; Chen Yet al. J. Am. Chem. Soc. 2020, 142, 12760-12766; Fan Z X et al. Small 2016, 12, 3908-3913; Han S B et al. Nat. Commun. 2020, 11, 552). The metastable 4H copper phase has been recently found to exhibit much higher electrocatalytic activities and ethylene selectivity in the CO₂ reduction reaction (Chen Y et al. J. Am. Chem. Soc. 2020. 142, 12760-12766). Thus, polytypism can significantly influence catalytic behavior.

One of the most exciting families of crystalline materials that commonly feature polytypism are Zintl-Klemm compounds, having structures comprised of layers of main group elements that are separated by large electropositive cations (Luo H X et al. Proc. Natl. Acad. Sci. U.S.A. 2015, 112, 1-7; Bojin M D et al. Helv. Chim. Acta 2003, 86, 1653-1682; Bojin M D et al. Helv. Chim. Acta 2003, 86, 1683-1708; Brown S R et al. Chem. Mat. 2006, 18, 1873-1877; Evans M J et al. Phys. Rev. B: Condens, Matter Mater. Phys. 2009, 80, 064514; Kim S J et al. New Cryst. Struct. 2008, 223, 325-326; Kim S J et al. Chem. Mat. 1999, 11, 3154-3159; Owens-Baird B et at. J. Am. Chem. Sec. 2020, 142, 2031-2041; Owens-Baird B et al. Chem. Mat. 2019, 31, 3407-3418). These materials exhibit a wealth of exotic physical properties including axis-dependent conduction polarity in NaSn₂As₂ (He B et al. Nat. Mater. 2019, 18, 568-572), reversible hydrogen absorption in SrGa₂ (Wenderoth P et al. Inorg. Chem. 2013, 52, 10525-10531), and the topological insulating properties of BaSn₂ (Kim S J et al. New Cryst. Struct. 2008, 223, 325-326). Some examples of polytypism in these Zintl-Klemm phases include CaGe₂, which can form a 2-layer and 6-layer polytype, as well as CaAlSi that can crystallize into 1-, 5-, or 6-layers per unit cell (Evans M J et al. Phys. Rev. B: Condens. Matter Mater. Phys. 2009, 80, 064514; Cultrara N D et al. Chem. Mat. 2018, 30, 1335-1343; Sagayama H et al. J. Phys. Soc. Jpn. 2006, 75, 043713).

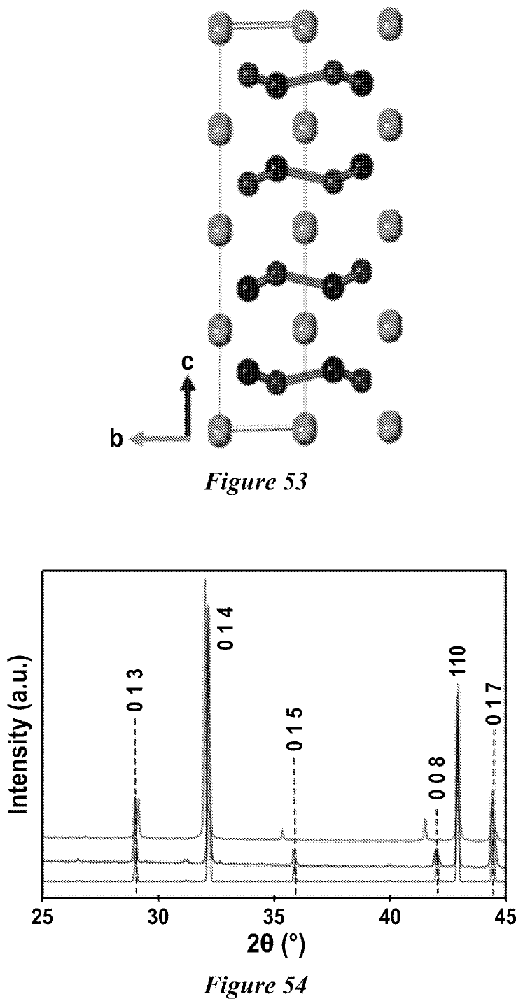

Recently, it was discovered that the transition metal-free layered Zintl-Klemm compound, BaGa₂, shows remarkable catalytic activities for the hydrogenation of phenylacetylene into styrene and ethylbenzene, that are within an order of magnitude of Pd-based compounds (Hodge K L et al. J. Am. Chem, Soc. 2019, 141, 19969-19972). The crystal structure of BaGa₂ comprises isolated layers of covalently bonded Ga⁻ honeycomb networks, in which every Ga atom has formally 7 valence electrons, ignoring the weak intralayer Ga—Ga π-bonding. It was hypothesized that the presence of 7 valence electrons would make these phases acidic, thereby promoting the adsorption of both H₂ and alkynes. The catalytic properties of other structurally and electronically similar transition metal-free layered intermetallics (discussed above) have been investigated. One such material is CaGaGe. This material was first discovered in 1989 and is comprised of puckered honeycomb [GaGe]²⁻ layers having an alternating BN-like arrangement, with each layer separated by Ca²⁺ (Czybulka A et al. Allg. Chem. 1989, 579, 151-157). According to Zintl-Klemm electron counting rules, formally each Ga⁻ and Ge⁻ would have 7 and 8 valence electrons, respectively. CaGaGe adopts a 4H YPtAs-type crystal. structure type, in which the Ga atoms in neighboring layers tilt towards each other to partially stabilize the lack of an octet, although the interlayer Ga—Ga distance is too large (3.57 Å) to form a covalent bond (FIG. 53 and FIG. 54) (Czybulka A et al. Allg. Chem. 1989, 579, 151-157), Subsequent transmission electron microscopy studies suggested that CaGaGe could form a more complex stacking sequence, although the structure was never fully determined (Evans M J et al. Phys. Rev. B: Condens. Matter Mater. Phys. 2009, 80, 064514). Herein, a new, 13-layer trigonal (13T) polytype of CaGaGe, that is an air-stable, highly active heterogeneous catalyst for the hydrogenation of alkynes, with consistently greater activities than the 4H polytype is elucidated.

### Experimental Methods

X-Ray and Neutron Diffraction: X-Ray Diffraction data was obtained using a Bruker D8 X-ray powder diffractometer (sealed Cu X-ray tube, 40 kV and 40 mA) equipped with a Lynxeye XE-T position-sensitive detector. The data were collected with an incident beam monochromator (Johansson type SiO₂-crystal) that selects only Cu K_{α1} radiation (λ=1.5406 Å). Time-of-flight neutron diffraction data were collected on the POWGEN beamline at the Spallation Neutron Source at Oak Ridge National Laboratory at ambient temperature using both λ=0.8 Å and λ=2.665 Å. Rietveld refinements of X-Ray and Neutron Diffraction data were carried out using the TOPAS-Academic, (Version 6) software package to determine the crystal structure.

X-Ray Photoelectron Spectroscopy was done using a Kratos Ultra X-ray photoelectron spectrometer with a monochromated Al X-ray source. Air-free powders were affixed to carbon tape on a sample holder in an Ar-filled glove box and loaded into an air-free transfer tool. Energy calibration was done using the C 1 s peak at 284.8 eV.

13T CaGaGe was synthesized by stoichiometric arc melting of the elements. 4H CaGaGe was synthesized by annealing 13T CaGaGe that was sealed in quartz tubes at 500° C. for 18 hours.

Catalytic hydrogenations were performed by initially grinding the appropriate polytype of CaGaGe in mortars and pestles and adding approximately 20 mg of the materials to glass vials. Approximately 2.5 mL of solvent (dimethylformamide or n-butanol) was added followed by 1.8 mmol of substrate. The glass vials were then placed in Parr reactors and the reactors were sealed. The reactors were then taken out of the glove box and the desired pressure of H₂ was added followed by placing the reactors on the heating elements. In samples where no catalyst was added, less than 5% conversion was observed.

### Results and Discussion

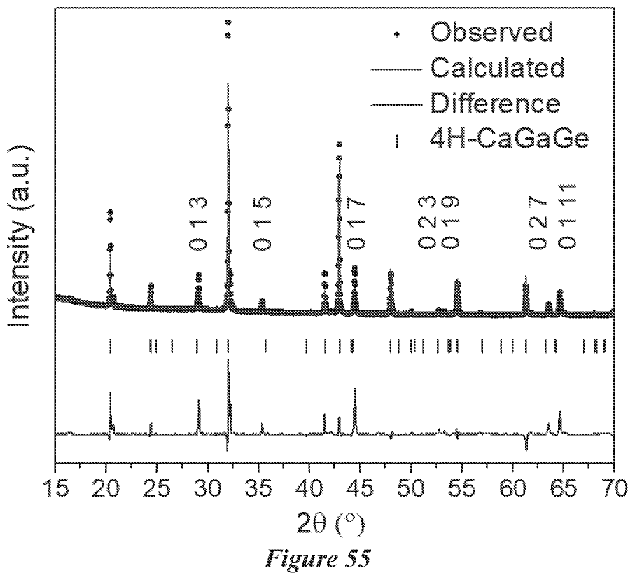

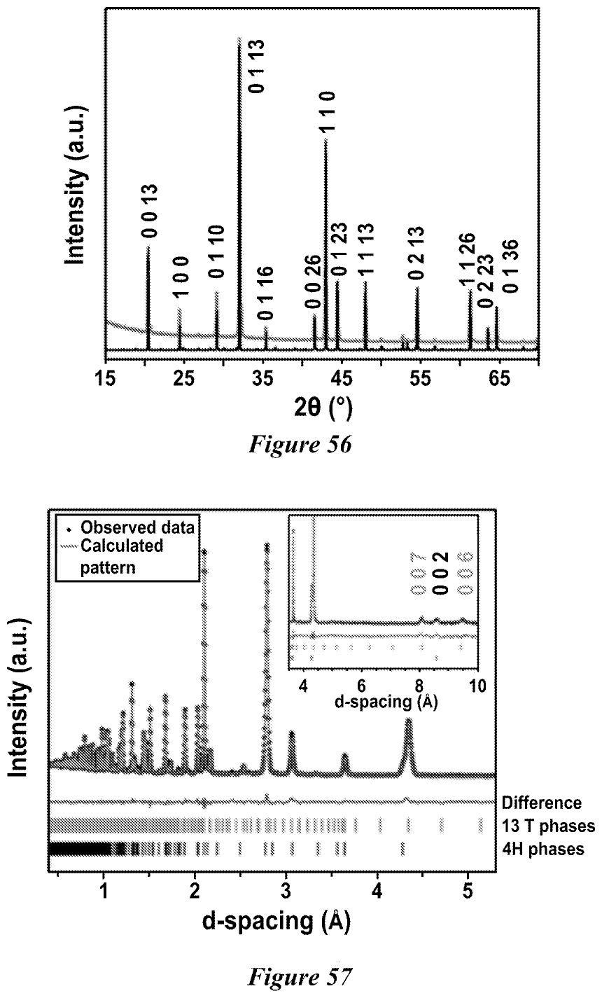

CaGaGe was synthesized by arc-melting stoichiometric amounts of the elements and initial Rietveld refinements were performed on in-house X-ray diffraction (XRD) data. All attempts to fit the powder pattern to the reported 4H P6₃/mm crystal structure were unsuccessful (FIG. 53, FIG. 54, FIG. 55). Specifically, when assuming a 4H polytype, all of the Okl reflections where C is odd are shifted to either higher or lower 2θ. However, the observed intensity of every reflection is close to the intensity predicted with this 411 polytype, indicating that the majority phase has a similar structure but has a different stacking sequence. Indeed, the d-spacing of the Okt reflections precisely match to a 13-layer trigonal or hexagonal unit cell (FIG. 56). The 4H CaGaGe phase is present as a minor impurity (˜15 wt %) that appears as shoulders on the most intense 13-layer reflections, shifted to a slightly larger 2θ. The 13-layer phase transforms into the 4H polytype upon annealing at 500° C. for 18 h (FIG. 54).

A time of flight (TOF) neutron diffraction powder pattern of CaGaGe was collected to elucidate the structure of this 13-layer phase since the neutron scattering factors of Ga and Ge are considerably different (Ga=6.83, Ge=8.6) compared to the similar X-ray form factors of these neighboring elements (Sears V F. Neutron News 1992, 3, 26-37). Rietveld refinement was able to convincingly establish a 13-layer unit cell as the majority phase (FIG. 57). The most visually straightforward evidence of the majority phase having a 13-layer sequence is the presence of the 006 and 007 reflections at large d-spacings (FIG. 57 inset). Between those reflections the 002 reflection of the 4H minority phase is apparent.

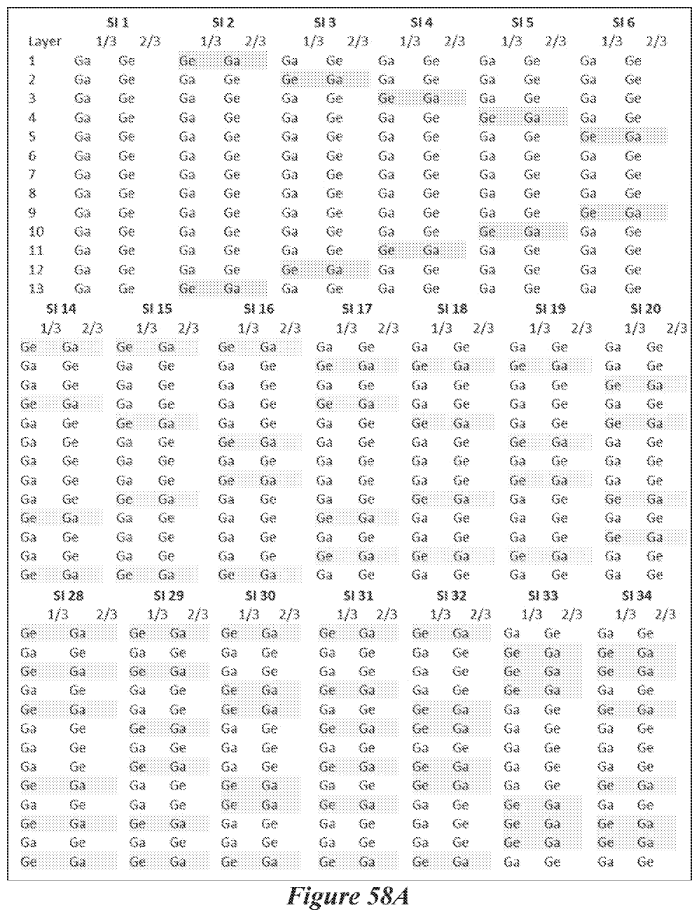

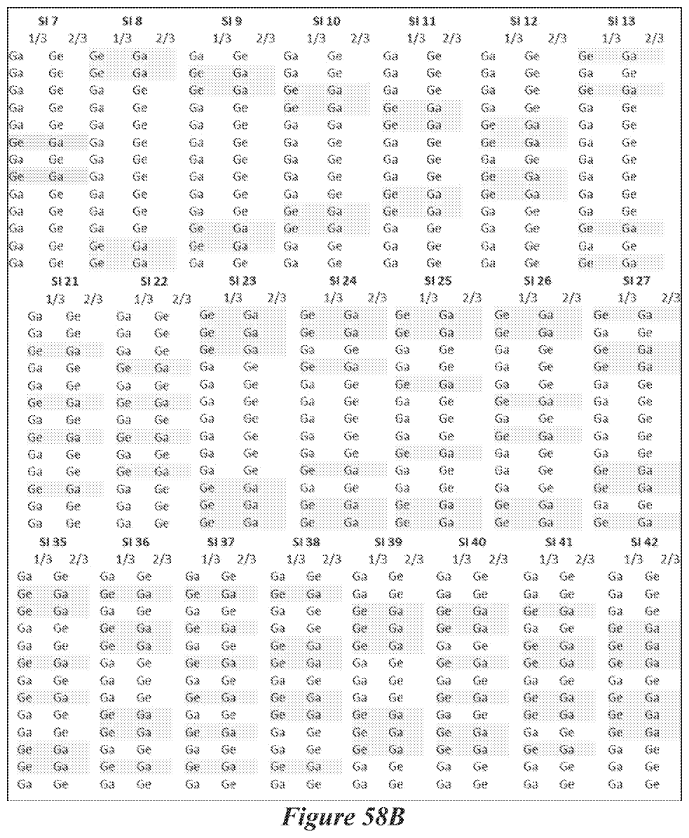

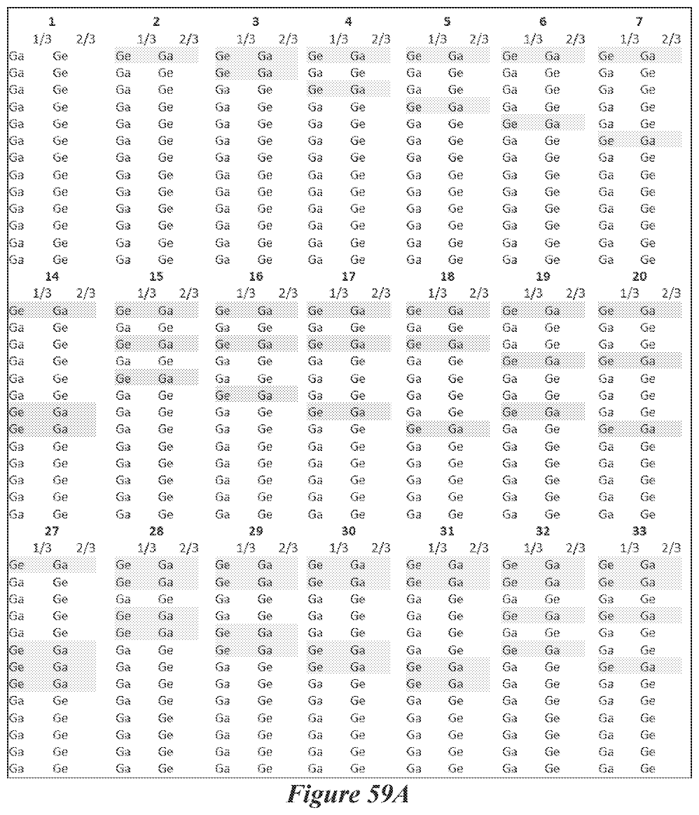

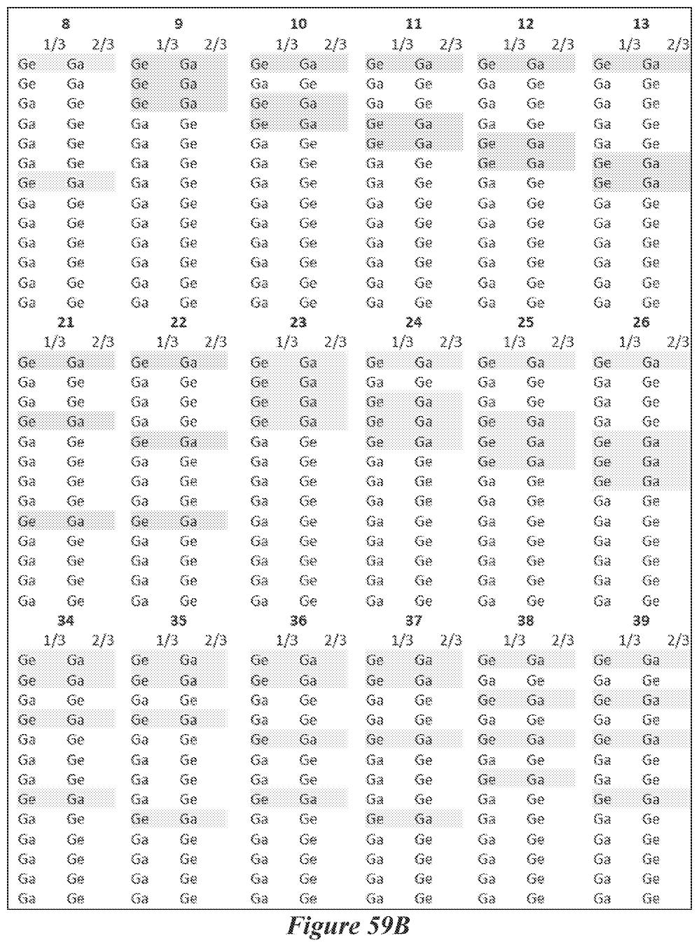

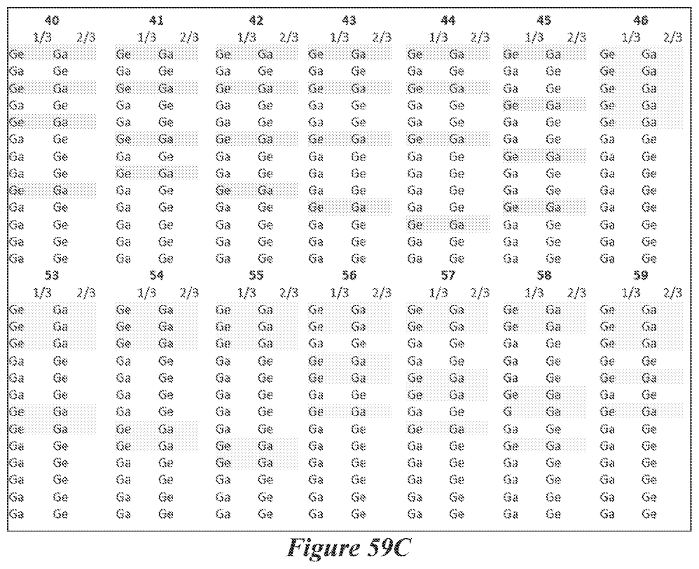

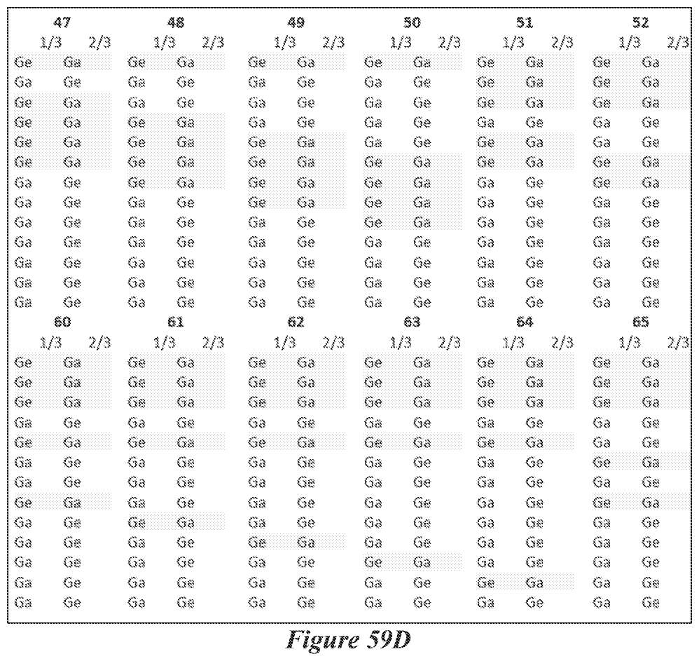

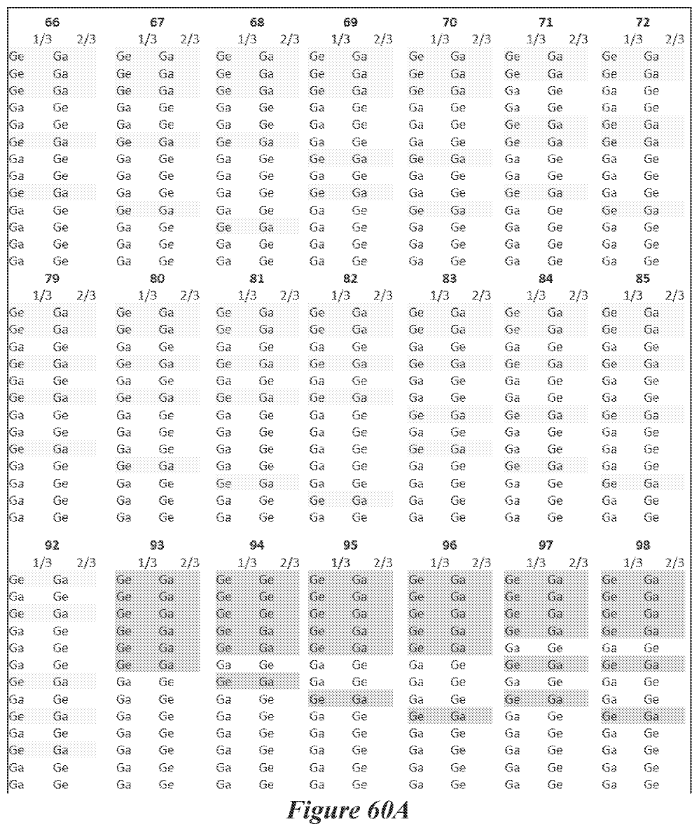

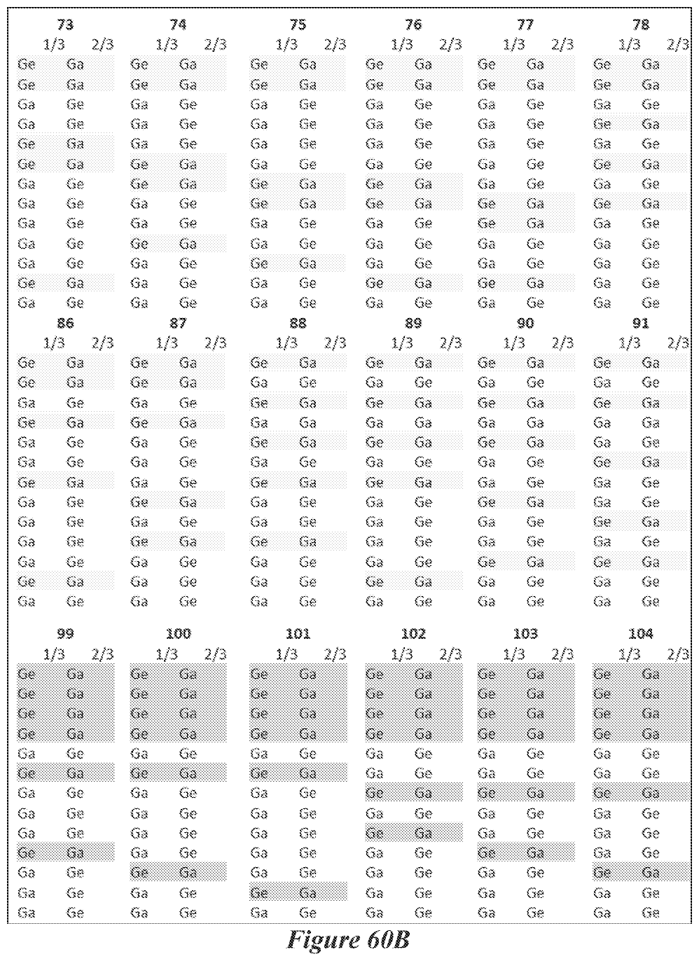

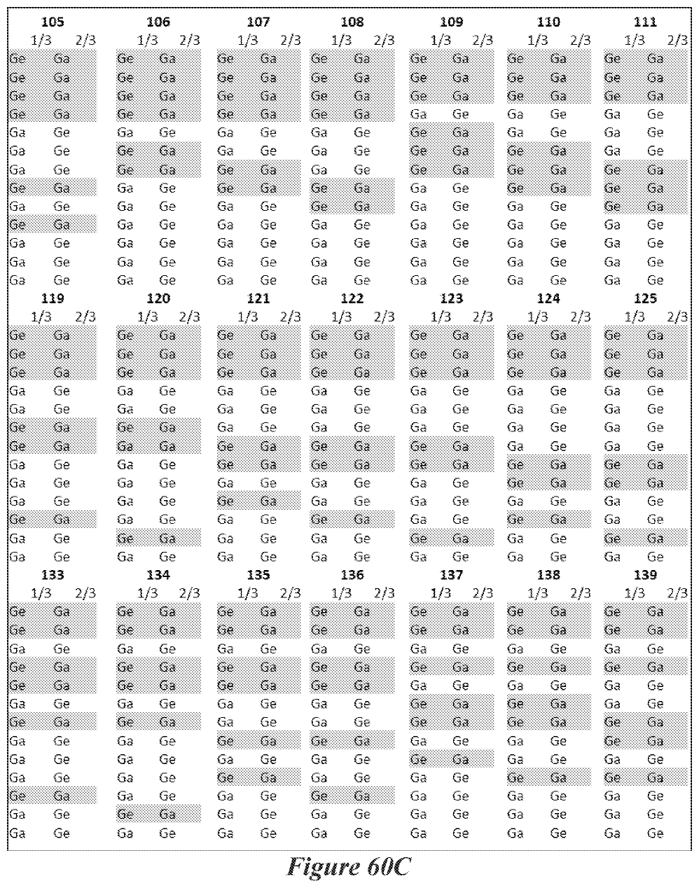

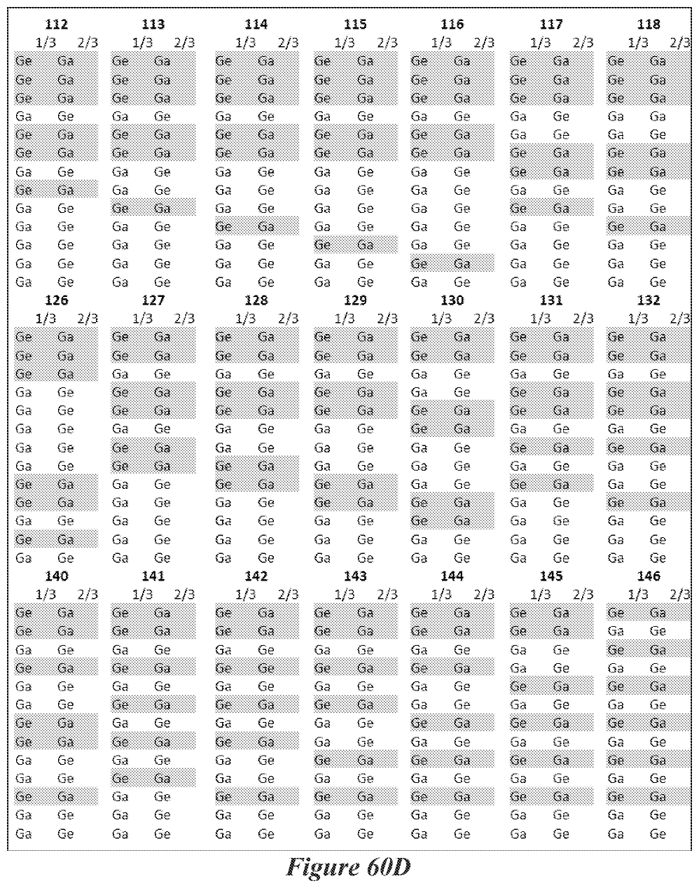

To fully elucidate the space group and structure of the 13-layer phase, many different structures were attempted with Rietveld analysis. First, the presence of the intense 0 0 13 reflection, where l is odd requires a space group with no systematic absences, thereby narrowing the number of possibilities down to 16. Of these 16, puckered honeycomb layers with alternating Ga and Ge can only exist in the P6̅ and P3 space groups. In the P6̅ space group, the presence of a mirror plane halfway up the unit cell causes the 1-6^{th} and 8^{th}-13^{th} layers to be mirror images of each other. However, the middle 7th layer must be planar. Assuming that the Ga will occupy either the 2h (⅓, ⅔, z) or the 2i (⅔, ⅓, z) position in each layer with the Ge occupying the other site, will lead to 42 unique possibilities in this space group (FIG. 58A-FIG. 58B). In the P3 space group, every layer is independent, as there are no mirror planes in the structure. This leads to 146 distinct possibilities assuming that the Ga in each layer occupies either the 1b (⅓, ⅔, z) or the 1c (⅔, ⅓, z) (FIG. 59A-FIG. 60D).

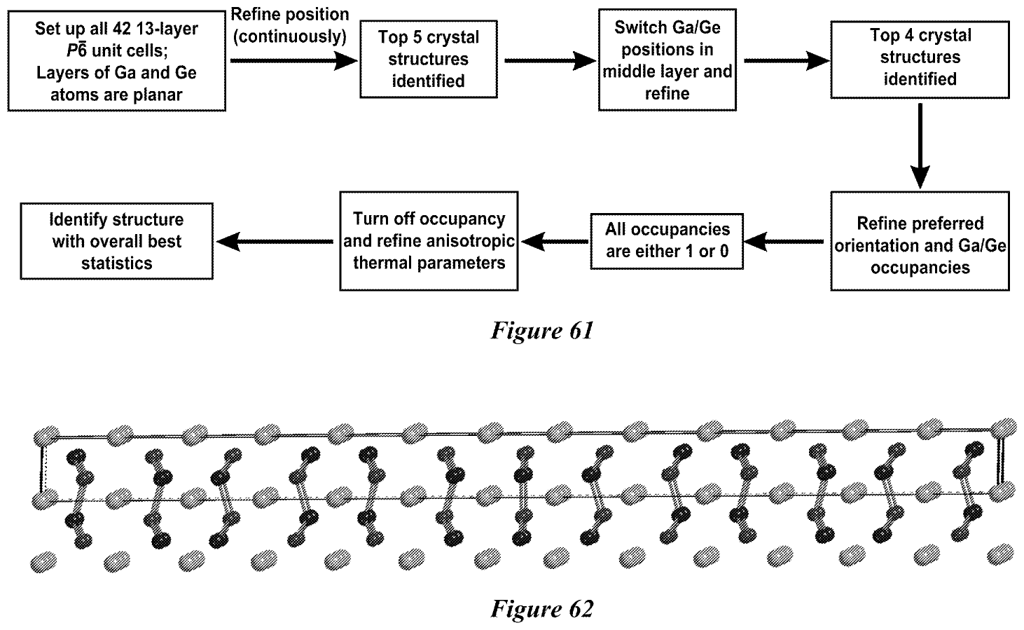

The P6̅ space group was initially considered. The refinement procedure is described in FIG. 61. To determine the most probable structure, initial refinements of all 42 unique crystal structures were performed, assuming the Ga and Ge atoms are planar within each layer. The Ga and Ge positions were then allowed to refine, in all cases creating buckling within the honeycomb Ga—Ge layers. The refinement was continued with the five crystal structures that produced the best statistics. In the best five crystal structures, the positions of gallium and germanium in the central, planar layer, were then switched to determine which produced better statistics. By allowing the positions in the middle layer to change and keeping the structures with the better statistics, two of the structures were then identical, so one was thrown out. The fractional occupancies were then allowed to refine freely for each structure, this produced almost no changes in the overall refinement. From this point, there was one clear crystal structure that produced the best statistics (FIG. 62). Considering that the relative intensities of each reflection in the pattern was very close to the 4H-CaGaGe unit cell, the refined P6̅ crystal structure was unsurprisingly very similar. For most of the structure (layers 1-5; 9-13) the Ga and Ge atoms switch sites every two layers, and the Ga atoms in neighboring layers (1,13; 2,3; 4,5; 9,10; 11,12) tilt towards each other to partially stabilize the lack of an octet.

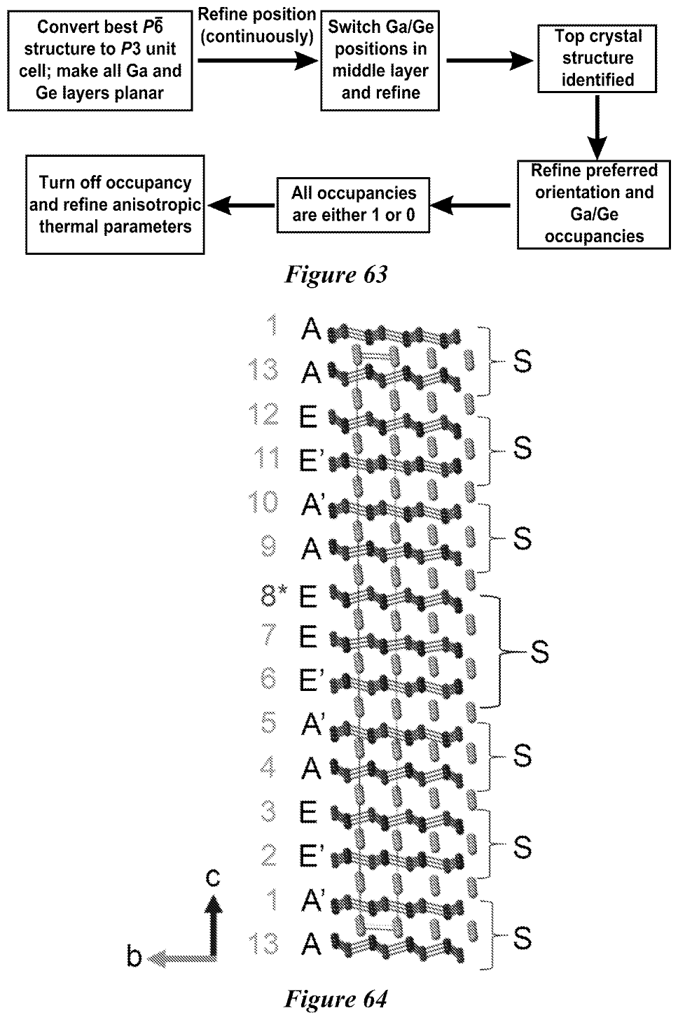

While the final P6̅ structure did produce a respectable refinement (R_{wp}=2.94%, GOF=5.72), DFT calculations of the formation energies indicated that having a planar middle layer is unstable by 0.13 eV, compared to puckering. Thus, these calculations indicate that the trigonal P3 space group is the more realistic structure. Rather than testing all 146 possibilities, the stacking sequence from the best P6̅ refinement was chosen and then converted and refined as a P3 unit cell using a revised procedure (FIG. 63). A slight improvement in refinement statistics (R_{wp}=2.88%, GOF=5.62) compared to P6̅, combined with the lower predicted formation energy indicated this to be a slightly better model. Refinement results are in Table 8-Table 9.

TABLE 8
Neutron Refinement of CaGaGe with
P3 structure type atomic parameters

| Atom | Site | x | y | z | U11 | U33 |
|---|---|---|---|---|---|---|
| Ca1 | 1a | 0 | 0 | 0.0011(10) | 0.0107(18) | 0.00598(20) |
| Ca2 | 1a | 0 | 0 | 0.07736(11) | 0.0107(18) | 0.00598(20) |
| Ca3 | 1a | 0 | 0 | 0.1534(12) | 0.0107(18) | 0.00598(20) |
| Ca4 | 1a | 0 | 0 | 0.2307(11) | 0.0107(18) | 0.00598(20) |
| Ca5 | 1a | 0 | 0 | 0.3126(7) | 0.0107(18) | 0.00598(20) |
| Ca6 | 1a | 0 | 0 | 0.384(8) | 0.0107(18) | 0.00598(20) |
| Ca7 | 1a | 0 | 0 | 0.4613(10) | 0.0107(18) | 0.00598(20) |
| Ca8 | 1a | 0 | 0 | 0.5381(9) | 0.0107(18) | 0.00598(20) |
| Ca9 | 1a | 0 | 0 | 0.6162(10) | 0.0107(18 | 0.00598(20) |
| Ca10 | 1a | 0 | 0 | 0.6921(8) | 0.0107(18) | 0.00598(20) |
| Ca11 | 1a | 0 | 0 | 0.7705(9) | 0.0107(18) | 0.00598(20) |
| Ca12 | 1a | 0 | 0 | 0.8459(9) | 0.0107(18) | 0.00598(20) |
| Ca13 | 1a | 0 | 0 | 0.9242(11) | 0.0107(18) | 0.00598(20) |
| GaL1 | 1c | ⅔ | ⅓ | 0.0314(7) | 0.0268(17) | 0.00280(14) |
| GeL1 | 1b | ⅓ | ⅔ | 0.0423(6) | 0.0144(8) | 0.00346(13) |
| GeL2 | 1c | ⅔ | ⅓ | 0.1122(5) | 0.0144(8) | 0.00346(13) |
| GaL2 | 1b | ⅓ | ⅔ | 0.1232(8) | 0.0268(17) | 0.00280(14) |
| GeL3 | 1c | ⅔ | ⅓ | 0.1949(6) | 0.0144(8) | 0.00346(13) |
| GaL3 | 1b | ⅓ | ⅔ | 0.1840(8) | 0.0268(17 | 0.00280(14) |
| GeL4 | 1b | ⅓ | ⅔ | 0.2641(5) | 0.0144(8) | 0.00346(13) |
| GaL4 | 1c | ⅔ | ⅓ | 0.2767(7) | 0.0268(17) | 0.00280(14) |
| GeL5 | 1b | ⅓ | ⅔ | 0.3463(5) | 0.0144(8) | 0.00346(13) |
| GaL5 | 1c | ⅔ | ⅓ | 0.3331(4) | 0.0268(17) | 0.00280(14) |
| GeL6 | 1c | ⅔ | ⅓ | 0.4173(6) | 0.0144(8) | 0.00346(13) |
| GaL6 | 1b | ⅓ | ⅔ | 0.4292(8) | 0.0268(17) | 0.00280(14) |
| GaL7 | 1b | ⅓ | ⅔ | 0.4934(5) | 0.0268(17) | 0.00280(14) |
| GeL7 | 1c | ⅔ | ⅓ | 0.5001(7) | 0.0144(8) | 0.00346(13) |
| GaL8 | 1b | ⅓ | ⅔ | 0.5719(6) | 0.0268(17) | 0.00280(14) |
| GeL8 | 1c | ⅔ | ⅓ | 0.5829(8) | 0.0144(8) | 0.00346(13) |
| GeL9 | 1b | ⅓ | ⅔ | 0.6537(8) | 0.0144(8) | 0.00346(13) |
| GaL9 | 1c | ⅔ | ⅓ | 0.6616(5) | 0.0268(17) | 0.00280(14) |
| GaL10 | 1c | ⅔ | ⅓ | 0.72460(8) | 0.0268(17) | 0.00280(14) |
| GeL10 | 1b | ⅓ | ⅔ | 0.7363(6) | 0.0144(8) | 0.00346(13) |
| GeL11 | 1c | ⅔ | ⅓ | 0.8060(5) | 0.0144(8) | 0.00346(13) |
| GaL11 | 1b | ⅓ | ⅔ | 0.8159(5) | 0.0268(17) | 0.00280(14) |
| GeL12 | 1c | ⅔ | ⅓ | 0.8886(5) | 0.0144(8) | 0.00346(13) |
| GaL12 | 1b | ⅓ | ⅔ | 0.8767(8) | 0.0268(17) | 0.00280(14) |
| GaL13 | 1c | ⅔ | ⅓ | 0.9711(6) | 0.0268(17) | 0.00280(14) |
| GeL13 | 1b | ⅓ | ⅔ | 0.9587(6) | 0.0144(8) | 0.00346(13) |

TABLE 9
Neutron powder refinement results from POWGEN time of
flight data using both the 13T and 4H CaGaGe structures
Phase I - 13T CaGaGe

| Chemical Formula | CaGaGe |
|---|---|
| Formula Weight (g/mol F.U.) | 182.43 |
| Crystal System | Trigonal |
| Space Group | P₃ |
| a (Å) | 4.20693(7) |
| c (Å) | 56.4561(12) |
| V (Å³) | 865.31(3) |
| Phase Fraction | 85.3% |

Phase II - 4H CaGaGe

| | |
|---|---|
| Chemical Formula | CaGaGe |
| Formula Weight (g/mol F.U.) | 182.43 |
| Crystal System | Hexagonal |
| Space Group | P63/mmc |
| a (Å) | 4.2060(5) |
| c (Å) | 17.1116(24) |
| V (Å³) | 262.16(7) |
| Phase Fraction | 14.7% |

Refinement conditions

| | |
|---|---|
| Temperature (K) | 300 |
| Time of flight range (μs) | 11000-90000 |
| R_{wp} | 2.88% |
| Goodness of fit | 5.616 |

Thus, a combination of Rietveld refinements and DFT calculations indicated that the polytype crystallizes into a P3 space group. The refined 13T-CaGaGe P3 crystal structure shown in FIG. 64 is nearly identical to the 4H-CaGaGe, with the exception of “layer 8”. For the rest of the structure, the position of the Ga and Ge atoms switch every two layers. The Ga atoms pucker towards each other to partially stabilize the lack of an octet, but again the average Ga—Ga interlayer distance of 3.45(14) Å is too large to indicate full covalent bonding. Layer 8 deviates from the 4H stacking sequence and lacks any interlayer Ga−Ga stabilization. In other words, the 13 layer unit cell can be thought of as having an overall repeat unit of 3×(4H-CaGaGe layers) capped by one CaGaGe layer that deviates from the stacking sequence.

Considering how close the 13-layer structure is to the original 4H unit cell, one could envision that other polytypes with 4n+1 layers could exist. DFT calculations indicated there to be negligible difference in formation energy normalized per formula unit between a 13-laver polytype and other structurally similar polytypes with 5, 9, 17, or 4n+1 layers. Regardless, no evidence for such structures was observed in the X-ray or neutron diffraction patterns. Nevertheless, this 13T phase is metastable and can only be accessed from the fast cooling achieved via arc-melting. This is the first example of a crystal structure with a 13-layer polytype in any crystalline material including materials known to have a plethora of polytypes (zeolites, SiC, etc. . . . ). The only previously claim of a 13-layer unit cell was in Ba₁₈Ti₅₄Nb₂O₁₃₂, although its atomic structure remains undetermined (Roth R S et al. J. Solid State Chem. 1987, 68, 330-339).

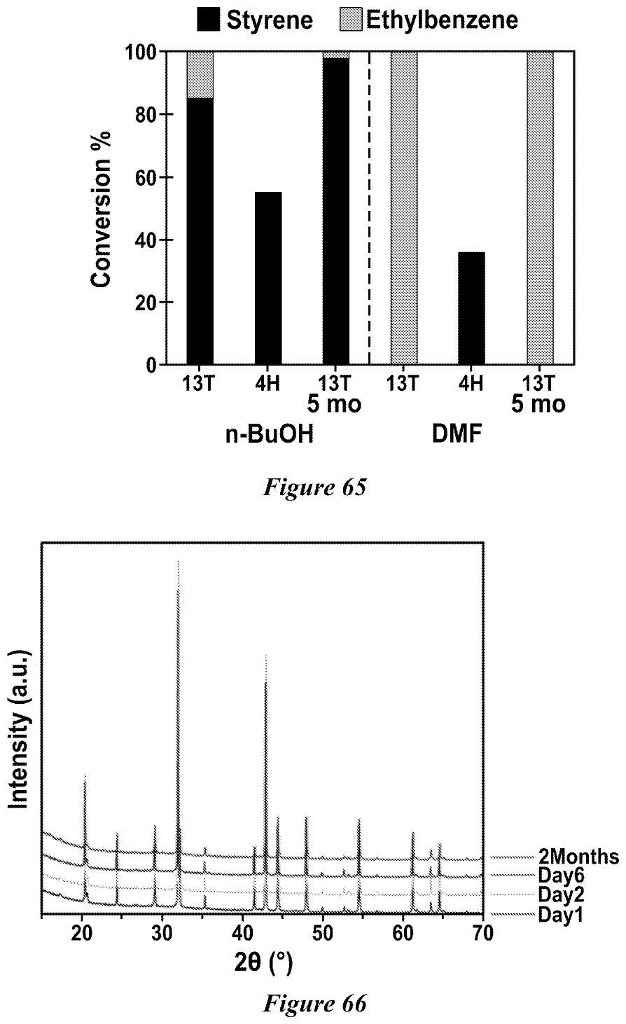

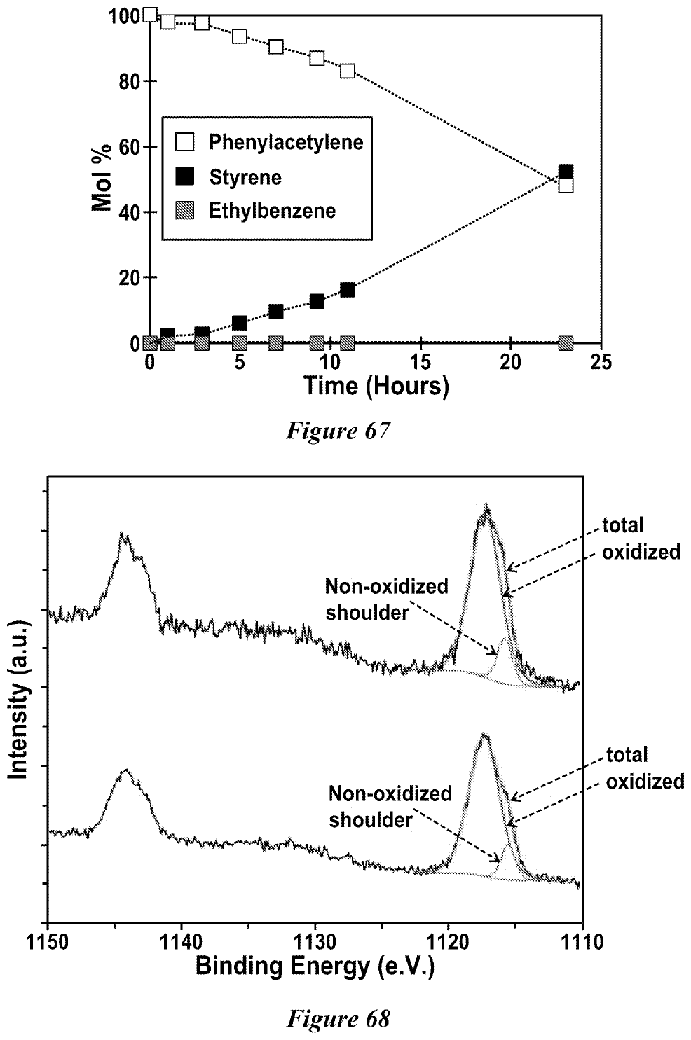

The catalytic activities of the 13T and 4H phases for the alkyne hydrogenation of phenylacetylene to styrene and ethylbenzene were first evaluated in both dimethylformamide (DMF) and n-butanol (n-BuOH) at 51 bar H₂ (FIG. 65), After 24 h, 13T-CaGaGe completely hydrogenates phenylacetylene to ethylbenzene in DMF and shows the more selective hydrogenation to styrene in n-BuOH. Surprisingly, 13T-CaGaGe consistently shows much greater conversion than the 4H-CaGaGe phase, despite having a nearly identical structure and particle sizes. This observation is quite robust, as the enhanced catalytic activity in 13T-CaGaGe was observed across three different synthetic batches of both polytypes. Additionally, 13T-CaGaGe has enhanced air-stability compared to the other Zintl-Klemm catalysts, including BaGa₂, which loses all activity upon immediate exposure to air (see above and Hodge K L et al. J. Am. Chem. Soc. 2019, 14.1, 19969-19972). After 5 months of air exposure, 13T-CaGaGe retained complete conversion with similar product mixtures in both DMF and n-BuOH, using these same reaction conditions. No new phases are observed in the diffraction pattern after this long-term air exposure, further evidencing the air-stability (FIG. 66). 13T-CaGaGe even exhibited some catalytic activity at 1 bar H₂ (FIG. 67). The time-dependent conversion in IMF shows >50% conversion after 23 h. The Surface Specific Activity (SSA) was estimated by measuring the surface area per gram of catalyst (0.406 m² g⁻¹) with the Kr adsorption isotherm Braunauer-Emmett-Teller method along with the conversion vs time. Using the 23 h time point, the surface specific activity is estimated to be 315 h⁻¹, which is within an order of magnitude of Lindlar's catalyst (650 h⁻¹) (see above and Hodge K L et al. J. Am. Chem. Soc. 2019, 141, 19969-19972). The presence of an induction period is apparent in the time dependent conversion, which complicates further kinetic analysis, and makes the estimate of surface specific activity a lower bound. An induction period was also similarly observed in BaGa₂, but ICP-OES analysis of the supernatant indicated the amount of soluble Ga and Ge to be <1 ppm, suggesting that catalysis is not due to a soluble Ga based impurity.

The difference in activity between polytypes is unexpected. Since the 4H-CaGaGe phase is prepared via annealing the 13T phase at 500° C. under vacuum in fused silica, whether this procedure resulted in additional oxidation on the surface of the 4H-CaGaGe phase, thereby reducing catalytic activity, was explored. However, X-ray Photoelectron Spectroscopy (XPS) of the 13T and 4H powders show no differences in the degree of oxidation between the polytypes (FIG. 66). Both phases show a major Ga 2p_{3/2} and 2p_{1/2} at 1117.90 and 1144.5 eV, indicative of an oxidized Ga³⁺ along with shoulders at 1115.7 and 1142.7 eV, indicative of an anionic Ga surface species. The presence of this anionic Ga surface species was previously shown to correlate with catalytic activity in BaGa₂ (Hodge K L et al. J. Am. Chem. Soc. 2019, 141, 19969-19972). The relative area of the anionic Ga shoulders is nearly identical in both 4H-CaGaGe and 13T-CaGaGe, indicating that the degree of surface oxidation is very similar (FIG. 68). This implicates the minor differences in structure between the two polytypes to cause their disparate catalytic behavior. One possible explanation is that the catalytic sites originating from the single misfit layer in the 13T phase are much more active. Another possibility is that the subtle changes in Ga—Ge bond length leads to enhanced activity. The 13T-polytype does have, on average, slightly smaller Ga—Ge bond distances 2.507(23) Å, ranging from 2.458 to 2.541 Å compared to the 4H structure 2.530(3) A. In other heterogeneous catalyst systems, similar deviations in metal-metal bond lengths have been shown to significantly influence catalytic activity (Wexler R B et al. J. Am. Chem. Soc. 2018, 140, 4678-4683). Further investigations can conclusively pinpoint the exact nature of the active sites, the mechanism of hydrogenation, and the origin of the increased activity in the 13T polytype.

### Conclusion

In conclusion, this exotic, metastable 13T-CaGaGe, is not only the first 13-layer polytype, but also provides an additional example of the unique role that polytypism can have on catalytic performance, beyond Cu and Au. Its formation is highly reproducible and is the dominant phase that forms through >50 synthesis attempts. This highly active transition metal-free, air-stable catalyst paves the way for the systematic exploration of the reactivity, selectivity, robustness, and mechanistic understanding in Zintl-Klemm phases.

Other advantages which are obvious and which are inherent to the invention will be evident to one skilled in the art. It will be understood that certain features and sub-combinations are of utility and may be employed without reference to other features and sub-combinations. This is contemplated by and is within the scope of the claims. Since many possible embodiments may be made of the invention without departing from the scope thereof, it is to be understood that all matter herein set forth or shown in the accompanying drawings is to be interpreted as illustrative and not in a limiting sense.

The methods of the appended claims are not limited in scope by the specific methods described herein, which are intended as illustrations of a few aspects of the claims and any methods that are functionally equivalent are intended to fall within the scope of the claims.

Various modifications of the methods in addition to those shown and described herein are intended to fall within the scope of the appended claims. Further, while only certain representative method steps disclosed herein are specifically described, other combinations of the method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated.

1. A catalyst comprising a layered intermetallic compound comprising:
AM₁₊ₓM′₁₋ₓ
wherein
A is a group 2 metal, a group 3 metal, a lanthanide metal, or a combination thereof;
M is a group 13 metal;
M′ is a group 14 metal or a group 14 metalloid; and
x is from 0 to 1.

2. The catalyst of claim 1, wherein the catalyst comprises BaGa₂, BaGaGe, BaGaSn, BalnGe, CaGe₂, CaIn₂, CaGaGe, CaGaSi, SrGa₂, SrAl₂, BaAlSi, SrAlSi, YGa₂, LnGa₂ wherein Ln is a lanthanide metal, or a combination thereof.

3. The catalyst of claim 1, wherein the catalyst comprises BaGa₂, BaGaGe, BaGaSn, BalnGe, or a combination thereof.

4. (canceled)

5. The catalyst of claim 1, wherein the catalyst is not BaGa₂ or SrGa₂.

6. (canceled)

7. The catalyst of claim 1, wherein the catalyst comprises BaGaGe, BaGaSn, BalnGe, CaGe₂, CaIn₂, CaGaGe, CaGaSi, SrAl₂, BaAlSi, SrAlSi, YGa₂, LnGa₂ wherein Ln is a lanthanide metal, or a combination thereof.

8. The catalyst of claim 1, wherein when A is Ba or Sr, x is not 1.

9. The catalyst of claim 1, wherein A is selected from the group consisting of Ba, Sr, Ca, Lu, Y, La, Ce, Pr, Nd, Sm, Eu, Gd, Tb, Dy, Ho, Er, Tm, Yb, and combinations thereof.

10. The catalyst of claim 1, wherein A is selected from the group consisting of Ca, Ba, Sr Y, La, Eu, and combinations thereof.

11. (canceled)

12. (canceled)

13. (canceled)

14. The catalyst of claim 1, wherein M is selected from the group consisting of Al, Ga, In, and combinations thereof.

15. (canceled)

16. The catalyst of claim 1, wherein M′ is selected from the group consisting of Si, Ge, Sn, and combinations thereof.

17. The catalyst of claim 1, wherein x is 0 or 1.

18. (canceled)

19. The catalyst of claim 1, wherein the catalyst comprises BalnGe, CaIn₂, CaGaGe, CaGaSi, YGa₂, LaGa₂, EuGa₂, or a combination thereof.

20. The catalyst of claim 1, wherein the catalyst comprises CaGaGe, CaGaSi, YGa₂, or a combination thereof.

21. (canceled)

22. (canceled)

23. (canceled)

24. The catalyst of claim 1, wherein the catalyst comprises CaGaGe.

25. (canceled)

26. The catalyst of claim 1, wherein the catalyst is substantially free of transition metals.

27. The catalyst of claim 1, wherein the catalyst comprises a layered Zintl phase.

28. (canceled)

29. (canceled)

30. (canceled)

31. (canceled)

32. The catalyst of claim 1, wherein the catalyst comprises a layered Zintl-Klemm phase comprising a triel network, a half-filled pz orbital, no interlayer bonding in the main group framework, or a combination thereof.

33. (canceled)

34. The catalyst of claim 1, wherein the catalyst comprises a plurality of particles having an average particle size of from 4 nm to 50 microns; wherein the catalyst has an average surface area of from 0.1 m² per gram of catalyst (m²/g) to 400 m²/g, or a combination thereof.

35. (canceled)

36. (canceled)

37. (canceled)

38. The catalyst of claim 1, wherein the catalyst effectively catalyzes a hydrogenation reaction.

39-44. (canceled)

45. A method of hydrogenating a hydrogenation substrate, the method comprising: combining the hydrogenation substrate and hydrogen in the presence of a catalyst, thereby hydrogenating the hydrogenation substrate and forming a mixture, wherein the catalyst comprises the catalyst of claim 1.

46-70. (canceled)
